(19) United States
(12) Patent Application Publication
(10) Pub. No.: US 2024/0299492 A1
(43) Pub. Date: Sep. 12, 2024

(54) COMBINATION THERAPIES WITH SIRP ALPHA-BASED CHIMERIC PROTEINS

(71) Applicant: Shattuck Labs, Inc., Austin, TX (US)

(72) Inventors: Taylor SCHREIBER, Austin, TX (US); Suresh DE SILVA, Austin, TX (US); George FROMM, Austin, TX (US)

(21) Appl. No.: 18/279,451

(22) PCT Filed: Mar. 4, 2022
(86) PCT No.: PCT/US2022/018853

§ 371 Date: Aug. 30, 2023

Related U.S. Application Data
(60) Provisional application No. 63/308,304, filed on Feb. 9, 2022.
(60) Provisional application No. 63/157,324, filed on Mar. 5, 2021.

(51) Int. Cl. A61K 38/17; A61K 31/282; A61K 31/337; A61K 31/439; A61K 31/454; A61K 31/4745; A61K 31/513; A61K 31/519; A61K 31/635; A61K 31/69; A61K 31/704; A61K 31/706; A61K 31/7068; A61K 33/243; A61K 38/19; A61K 39/395; A61K 45/06; A61P 35/00

(52) U.S. Cl. CPC A61K 38/1774; A61K 31/282; A61K 31/337; A61K 31/439; A61K 31/454; A61K 31/4745; A61K 31/513; A61K 31/519; A61K 31/635; A61K 31/69; A61K 31/704; A61K 31/706; A61K 31/7068; A61K 33/243; A61K 38/191; A61K 39/3955; A61K 45/06; A61P 35/00

(57) ABSTRACT

The present disclosure relates to, inter alia, combinations of compositions which include chimeric proteins that find use in methods for treating disease, such as immunotherapies for cancer.

### PRIORITY

This application claims the benefit of, and priority to, U.S. Provisional Application Nos. 63/308,304, filed Feb. 9, 2022; and 63/157,324, filed Mar. 5, 2021, the contents of each which are hereby incorporated by reference in their entirety.

### TECHNICAL FIELD

The present disclosure relates to, inter alia, combinations of compositions which include chimeric proteins that find use in methods for treating disease, such as immunotherapies for cancer.

### DESCRIPTION OF THE TEXT FILE SUBMITTED ELECTRONICALLY

This application contains a sequence listing. It has been submitted electronically via EFS-Web as an ASCII text file entitled “SHK-045PC_116981-5045_ST25”. The sequence listing is 57,499 bytes in size, and was created on Mar. 2, 2022. The sequence listing is hereby incorporated by reference in its entirety.

### BACKGROUND

Combination therapies are very common in modern cancer treatment. However, combination therapies are highly unpredictable. For example, combination therapy may not be efficacious even when drug target pairs are validated. The obstacles faced by combination therapies include lack of efficacy, undesirable drug-drug interactions, drug toxicity of the combination, development of common underlying resistance mechanisms (e.g. drug effux pumps), inability to predict treatment efficacy, the need for additional biomarkers, etc. Although some combinations show a therapeutic benefit, the efficacy is observed in only a select group of cancers and usually in a minority of patients with those cancers. Therefore, more work is required to find new combination therapies to treat cancer.

### SUMMARY

Accordingly, in one aspect, the present disclosure provides a method for treating a cancer in a subject in need thereof comprising: administering to the subject a first pharmaceutical composition comprising a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain; and administering to the subject a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof.

In embodiments, the first pharmaceutical composition and the second pharmaceutical composition are administered simultaneously. In embodiments, the first pharmaceutical composition is administered after the second pharmaceutical composition is administered. In embodiments, the first pharmaceutical composition is administered before the second pharmaceutical composition is administered. In embodiments, the dose of the first pharmaceutical composition is less than the dose of the first pharmaceutical composition administered to a subject who has not undergone or is not undergoing treatment with the second pharmaceutical composition. In embodiments, the dose of the second pharmaceutical composition administered is less than the dose of the second pharmaceutical composition administered to a subject who has not undergone or is not undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition.

In one aspect, the present disclosure provides a method for treating a cancer in a subject comprising: administering to the subject a pharmaceutical composition comprising a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain; wherein the subject has undergone or is undergoing treatment with a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof.

In embodiments, the dose of the pharmaceutical composition administered to the subject is less than the dose of the pharmaceutical composition that is administered to a subject who has not undergone or is not undergoing treatment with the second pharmaceutical composition.

In one aspect, the present disclosure provides a method for treating a cancer in a subject comprising: administering to the subject a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; wherein the subject has undergone or is undergoing treatment with a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain.

In embodiments, the dose of the pharmaceutical composition provided to the subject is less than the dose of the pharmaceutical composition that is provided to a subject who has not undergone or is not undergoing treatment with the heterologous chimeric protein.

In any of the embodiments disclosed herein, the second pharmaceutical composition comprises a hypomethylating agent/epigenetic regulator. In embodiments, the hypomethylating agent/epigenetic regulator is selected from azacitidine, 5-aza-2′-deoxycytidine, suberoylanilide hydroxamic acid (saha), romidepsin, belinostat, panobinostat, and chidamide. In embodiments, the hypomethylating agent/epigenetic regulator is azacitidine.

Additionally or alternatively, in embodiments, the second pharmaceutical composition comprises a proteasomal inhibitor. In embodiments, the proteasomal inhibitor is selected from bortezomib, carfilzomib and ixazomib. In embodiments, the proteasomal inhibitor is bortezomib.

Additionally or alternatively, in embodiments, the second pharmaceutical composition comprises an anti-metabolite. In embodiments, the antimetabolite is selected from 5-fluorouracil (5-FU), capecitabine, floxuridine, cytarabine (ARA-C), gemcitabine, decitabine, and vidaza. In embodiments, the antimetabolite is cytarabine (ARA-C).

Additionally or alternatively, in embodiments, the second pharmaceutical composition comprises a DNA synthesis inhibitor. In embodiments, the DNA synthesis inhibitor is selected from 5-fluorouracil (5-FU), capecitabine, floxuridine, cytarabine (ARA-C), gemcitabine, decitabine, and vidaza. In embodiments, the DNA synthesis inhibitor is cytarabine (ARA-C) or 5-fluorouracil (5-FU).

Additionally or alternatively, in embodiments, the second pharmaceutical composition comprises an immune checkpoint inhibitor. In embodiments, the immune checkpoint inhibitor comprises an agent that inhibits a pathway selected from CTLA-4, PD-1 and PD-L1. In embodiments, the immune checkpoint inhibitor comprises an anti-PD-L1 antibody. In embodiments, the anti-PD-L1 antibody is selected from atezolizumab, durvalumab, avelumab, envafolimab, BMS-936559, CK-301, CS-1001, SHR-1316 (HTI-1088), CBT-502 (TQB-2450) and BGB-A333.

Additionally or alternatively, in embodiments, the second pharmaceutical composition comprises an anthracyline. In embodiments, the anthracycline is selected from daunorubicin, doxorubicin, epirubicin, idarubicin, mitoxantrone, and valrubicin. In embodiments, the anthracycline is doxorubicin.

Additionally or alternatively, in embodiments, the second pharmaceutical composition comprises a topoisomerase II inhibitor. In embodiments, the topoisomerase II inhibitor is selected from doxorubicin, epirubicin, valrubicin, daunorubicin, idarubicin, pitoxantrone, pixantrone, etoposide, teniposide, and amsacrine. In embodiments, the topoisomerase II inhibitor is doxorubicin.

Additionally or alternatively, in embodiments, the second pharmaceutical composition comprises an innate immune checkpoint inhibitor. In embodiments, the innate immune checkpoint inhibitor comprises an agent that target CD47-SIRPα interaction. In embodiments, the innate immune checkpoint inhibitor is selected from magrolimab, CC-90002 (Celgene), CC-95251 (Celgene), TTI-621 (Trillium Therapeutics), TTI-622 (Trillium Therapeutics), ALX148 (ALX Oncology), SRF231 (Surface Oncology), IBI188 (Innovent), AO-176 (Arch Oncology), BI 765063/OSE-172 (Boehringer Ingelheim/OSE Immunotherapeutics), TG-1801/NI_1701 (TG Therapeutics/Novimmune), TJC4 (I-Mab) and the SIRPα-Fc-CD40L chimeric protein.

Additionally or alternatively, in embodiments, the second pharmaceutical composition comprises a Bcl2 inhibitor. In embodiments, the Bcl2 inhibitor is selected from oblimersen, navitoclax (ABT-263), venetoclax (ABT-199), obatoclax mesylate (GX15-070), and AT-101. In embodiments, the Bcl2 inhibitor is venetoclax.

Additionally or alternatively, in embodiments, the second pharmaceutical composition comprises a protein neddylation inhibitor. In embodiments, the protein neddylation inhibitor is pevonedistat (MLN4924).

Additionally or alternatively, in embodiments, the second pharmaceutical composition comprises a microtubule-targeting agent. In embodiments, the microtubule-targeting agent is selected from paclitaxel, epothilone, docetaxel, discodermolide, vinblastine, vincristine, vinorelbine, vinflunine, dolastatins, halichondrins, hemiasterlins, and cryptophysin 52. In embodiments, the microtubule-targeting agent is paclitaxel.

Additionally or alternatively, in embodiments, the second pharmaceutical composition comprises a thymidylate synthase (TS) inhibitor. In embodiments, the thymidylate synthase (TS) inhibitor is selected from 5-fluorouracil (5-FU), 6-mercaptopurine (6-MP), capecitabine, cytarabine, floxuridine, fludarabine, gemcitabine, hydroxycarbamide, methotrexate, pemetrexed, phototrexate, raltitrexed, nolatrexed, ZD9331, and GS7904L. In embodiments, the thymidylate synthase (TS) inhibitor is 5-fluorouracil (5-FU).

Additionally or alternatively, in embodiments, the second pharmaceutical composition comprises a platinum drug. In embodiments, the platinum drug is selected from cisplatin, carboplatin, oxaliplatin, nedaplatin, heptaplatin and lobaplatin. In embodiments, the platinum drug is cisplatin. In embodiments, the platinum drug is oxaliplatin.

Additionally or alternatively, in embodiments, the second pharmaceutical composition comprises a topoisomerase I inhibitor. In embodiments, the topoisomerase I inhibitor is selected from camptothecin, belotecan topotecan, and irinotecan. In embodiments, the topoisomerase I inhibitor is irinotecan.

Additionally or alternatively, in embodiments, the second pharmaceutical composition comprises an anti-BCMA antibody. In embodiments, the anti-BCMA antibody is belantamab mafodotin. In embodiments, the anti-BCMA antibody is belantamab or C12A3.2.

Additionally or alternatively, in embodiments, the second pharmaceutical composition comprises an anti-CD38 antibody. In embodiments, the anti-CD38 antibody is selected from daratumumab and isatuximab. In embodiments, the anti-CD38 antibody is daratumumab.

Additionally or alternatively, in embodiments, the second pharmaceutical composition comprises an immunomodulatory imide drug (IMiD). In embodiments, the immunomodulatory imide drug (IMiD) is selected from apremilast, thalidomide, lenalidomide, and pomalidomide. In embodiments, the immunomodulatory imide drug (IMiD) is lenalidomide or pomalidomide.

Additionally or alternatively, in embodiments, the second pharmaceutical composition comprises an anti-SLAMF7 antibody. In embodiments, the anti-SLAMF7 antibody is elotuzumab.

Additionally or alternatively, in embodiments, the second pharmaceutical composition comprises an anti-CD123 antibody. In embodiments, the anti-CD123 antibody is talacotuzumab.

Additionally or alternatively, in embodiments, the second pharmaceutical composition comprises a reactivator of mutated p53. In embodiments, the reactivator of mutated p53 is Prima-1 or APR-246. In embodiments, the reactivator of mutated p53 is APR-246.

Additionally or alternatively, in embodiments, the second pharmaceutical composition comprises an anti-FOLR1 antibody. In embodiments, the anti-FOLR1 antibody is farletuzumab or mirvetuximab, including mirvetuximab soravtansine. In embodiments, the anti-FOLR1 antibody is farletuzumab.

Additionally or alternatively, in embodiments, the second pharmaceutical composition comprises azacitidine and/or venetoclax, optionally wherein the azacitidine and venetoclax are contained in two separate dosage units, which are administered together or separately.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising a hypomethylating agent/epigenetic regulator. In embodiments, the hypomethylating agent/epigenetic regulator is selected from azacitidine, 5-aza-2′-deoxycytidine, suberoylanilide hydroxamic acid (saha), romidepsin, belinostat, panobinostat, and chidamide.

In embodiments, the hypomethylating agent/epigenetic regulator is azacitidine.

In any of the embodiments disclosed herein, the heterologous chimeric protein comprises a first domain which comprises substantially the entire extracellular domain of SIRPα(CD172a) and/or a second domain which comprises substantially the entire extracellular domain of CD40L, OX40L, or LIGHT. In embodiments, the heterologous chimeric protein comprises: (a) a first domain comprising a portion of SIRPα(CD172a), (b) a second domain comprising a portion of CD40L, OX40L, or LIGHT, and (c) a linker comprising a hinge-CH2-CH3 Fc domain.

In any of the embodiments disclosed herein, the linker is a polypeptide selected from a flexible amino acid sequence, an IgG hinge region, and an antibody sequence. In embodiments, the linker comprises at least one cysteine residue capable of forming a disulfide bond and/or comprises a hinge-CH2-CH3 Fc domain. In embodiments, the linker comprises a hinge-CH2-CH3 Fc domain derived from IgG1 or IgG4, e.g., human IgG4 or human IgG4. In embodiments, the linker comprises an amino acid sequence that is at least 95% identical to the amino acid sequence of SEQ ID NO: 1, SEQ ID NO: 2, or SEQ ID NO: 3.

In embodiments, the first domain comprises an amino acid sequence that is at least 90%, or at least 93%, at least 95%, or at least 96%, or at least 98%, or at least 99% identical to the amino acid sequence of SEQ ID NO: 57.

In embodiments, the second domain comprises an amino acid sequence that is at least 90%, or at least 93%, at least 95%, or at least 96%, or at least 98%, or at least 99% identical to the amino acid sequence of SEQ ID NO: 58, SEQ ID NO: 59 or SEQ ID NO: 62. In embodiments, the second domain comprises an amino acid sequence that is at least 90%, or at least 93%, at least 95%, or at least 96%, or at least 98%, or at least 99% identical to the amino acid sequence of SEQ ID NO: 58.

In embodiments, the heterologous chimeric protein comprises an amino acid sequence that is at least 90%, or at least 93%, at least 95%, or at least 96%, or at least 98%, or at least 99% identical to the amino acid sequence of SEQ ID NO: 60, SEQ ID NO: 61, or SEQ ID NO: 63. In embodiments, the heterologous chimeric protein comprises an amino acid sequence that is at least 90%, or at least 93%, at least 95%, or at least 96%, or at least 98%, or at least 99% identical to the amino acid sequence of SEQ ID NO: 60.

In embodiments, the cancer is or is related to a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

### BRIEF DESCRIPTION OF THE FIGURES

FIG. 1A to FIG. 1D show schematic illustrations of Type I transmembrane proteins (FIG. 1A and FIG. 1B, left proteins) and Type II transmembrane proteins (FIG. 1A and FIG. 1B, right proteins). A Type I transmembrane protein and a Type II transmembrane protein may be engineered such that their transmembrane and intracellular domains are omitted and the transmembrane proteins' extracellular domains are adjoined using a linker sequence to generate a single chimeric protein. As shown in FIG. 1C and FIG. 1D, the extracellular domain of a Type I transmembrane protein, e.g., SIRPα(CD172a), and the extracellular domain of a Type II transmembrane protein, e.g., CD40L, and OX40L, are combined into a single chimeric protein. FIG. 1C depicts the linkage of the Type I transmembrane protein and the Type II transmembrane protein by omission of the transmembrane and intracellular domains of each protein, and where the liberated extracellular domains from each protein have been adjoined by a linker sequence. The extracellular domains in this depiction may include the entire amino acid sequence of the Type I protein (e.g., SIRPα(CD172a)) and/or Type II protein (e.g., CD40L, OX40L, LIGHT) which is typically localized outside the cell membrane, or any portion thereof which retains binding to the intended receptor or ligand. Moreover, the chimeric protein used in a method of the present disclosure comprises sufficient overall flexibility and/or physical distance between domains such that a first extracellular domain (shown at the left end of the chimeric protein in FIG. 1C and FIG. 1D) is sterically capable of binding its receptor/ligand and/or a second extracellular domain (shown at the right end of the chimeric protein in FIG. 1C and FIG. 1D) is sterically capable of binding its receptor/ligand.

FIG. 1D depicts adjoined extracellular domains in a linear chimeric protein wherein each extracellular domain of the chimeric protein is facing “outward.”

FIG. 2A and FIG. 2B show the effect of azacitidine (an hypomethylating agent) on the in vitro phagocytosis-stimulating activity of the SIRPα-Fc-CD40L chimeric protein. A bar graph of the extent of phagocytosis of the K652 human chronic myelogenous leukemia (CML) cells (FIG. 2A) or the Kasumi-3 human acute myelocytic leukemia (AML) cells (FIG. 2B) by human macrophages as measured by flow cytometry is shown. Dotted line shows the extent of phagocytosis of the cancer cells treated with buffer only control.

FIG. 3A and FIG. 3B show the effect of bortezomib (a proteasomal inhibitor (PSI)) on the in vitro phagocytosis-stimulating activity of the SIRPα-Fc-CD40L chimeric protein. A bar graph of the extent of phagocytosis of the human macrophages of the MM1R human multiple myeloma (MM) cells (FIG. 3A), or the ARD1 human multiple myeloma (MM) cells (FIG. 3B) by human macrophages as measured by flow cytometry is shown. Dotted line shows the extent of phagocytosis of the cancer cells treated with buffer only control.

FIG. 4 shows the effect of venetoclax (a Bcl-2 inhibitor) on the in vitro phagocytosis-stimulating activity of the SIRPα-Fc-CD40L chimeric protein. A bar graph of the extent of phagocytosis of the human macrophages of the K652 human chronic myelogenous leukemia (CML) cells by human macrophages as measured by flow cytometry is shown. Dotted line shows the extent of phagocytosis of the cancer cells treated with buffer only control.

FIG. 5A and FIG. 5B show the effect of an anti-BCMA antibody (clone C12A3.2) on the in vitro phagocytosis-stimulating activity of the SIRPα-Fc-CD40L chimeric protein. A bar graph of the extent of phagocytosis of the human macrophages of the human macrophages of the KM28PE human multiple myeloma (MM) cells (FIG. 5A) or KM12B human multiple myeloma (MM) cells (FIG. 5B) by human macrophages as measured by flow cytometry is shown. Dotted line shows the extent of phagocytosis of the cancer cells treated with buffer only control.

FIG. 6 shows the effect of daratumumab (an anti-CD38 antibody) on the in vitro phagocytosis-stimulating activity of the SIRPα-Fc-CD40L chimeric protein. A bar graph of the extent of phagocytosis of the human macrophages of the ARD1 human multiple myeloma (MM) cells by human macrophages as measured by flow cytometry is shown. Dotted line shows the extent of phagocytosis of the cancer cells treated with buffer only control.

FIG. 7 shows the effect of the SIRPα-Fc-CD40L chimeric protein on the in vitro phagocytosis-stimulating activity of pomalidomide. A bar graph of the extent of phagocytosis of the human macrophages of the KMS12B human multiple myeloma (MM) cells by human macrophages as measured by flow cytometry is shown. Dotted line shows the extent of phagocytosis of the cancer cells treated without activated T cells.

FIG. 8 shows the effect of elotumab (an anti-SLAM7 antibody) on the in vitro phagocytosis-stimulating activity of the SIRPα-Fc-CD40L chimeric protein. A bar graph of the extent of phagocytosis of the human macrophages of the ARD1 human multiple myeloma (MM) cells by human macrophages as measured by flow cytometry is shown. Dotted line shows the extent of phagocytosis of the cancer cells treated with buffer only control.

FIG. 9 shows the effect an anti-FOLR1 antibody on the in vitro phagocytosis-stimulating activity of the SIRPα-Fc-CD40L chimeric protein. A bar graph of the extent of phagocytosis of the human macrophages of the ARD1 human multiple myeloma (MM) cells by human macrophages as measured by flow cytometry is shown. Dotted line shows the extent of phagocytosis of the cancer cells treated with buffer only control.

FIG. 10A to FIG. 10F show the in vivo anti-tumor efficacy of a combination of the SIRPα-Fc-CD40L chimeric protein with various chemotherapeutic agents in the CT26 colorectal carcinoma mouse allograft model. FIG. 10A shows the in vivo anti-tumor efficacy the SIRPα-Fc-CD40L chimeric protein, paclitaxel, or their combination. FIG. 10B shows the in vivo anti-tumor efficacy the SIRPα-Fc-CD40L chimeric protein, 5-fluorouracil, or their combination. FIG. 10C shows the in vivo anti-tumor efficacy the SIRPα-Fc-CD40L chimeric protein, irinotecan, or their combination. FIG. 10D shows the in vivo anti-tumor efficacy the SIRPα-Fc-CD40L chimeric protein, doxorubicin, or their combination. FIG. 10E shows the in vivo anti-tumor efficacy the SIRPα-Fc-CD40L chimeric protein, cisplatin, or their combination. FIG. 10F shows the in vivo anti-tumor efficacy the SIRPα-Fc-CD40L chimeric protein, oxaliplatin, or their combination.

FIG. 11A to FIG. 11D show the effect of azacitidine or pevonedistat (MLN4924) on the surface expression of CD47 (FIG. 11A and FIG. 11B) or calreticulin (FIG. 11C and FIG. 11D) in the K652 human chronic myelogenous leukemia (CML) cells (FIG. 11A and FIG. 11C), or the Kasumi-3 human acute myelocytic leukemia (AML) cells (FIG. 11B and FIG. 11D), as measured by flow cytometry.

FIG. 12A and FIG. 12B show the effect of APR-246 on the surface expression of p53 (FIG. 12A) and calreticulin (FIG. 12B).

FIG. 13A to FIG. 13C show the in vivo anti-tumor efficacy of combinations of the SIRPα-Fc-CD40L chimeric protein the various drugs in the A20 lymphoma cell allograft mouse model. FIG. 13A shows the in vivo anti-tumor efficacy of a combination of the SIRPα-Fc-CD40L chimeric protein with an anti-PD-L1 antibody. FIG. 13B shows the in vivo anti-tumor efficacy of a combination of the SIRPα-Fc-CD40L chimeric protein with cytarabine. FIG. 13C shows the in vivo anti-tumor efficacy of a combination of the SIRPα-Fc-CD40L chimeric protein with azacitidine and/or pevonedistat (MLN4924).

FIG. 14A shows that azacitidine and venetoclax increased the expression of the apoptosis marker Annexin-V in Kasumi-3 AML cells. FIG. 14B shows that azacitidine and venetoclax increased the expression of calreticulin in Kasumi-3 AML cells.to FIG. 14C shows that the SIRPα-Fc-CD40L chimeric protein combined with azacitidine and venetoclax enhanced macrophage-mediated phagocytosis of AML cells.

### DETAILED DESCRIPTION

The present disclosure is based, in part, on the discovery that the chimeric proteins comprising the extracellular, or effector, regions of Signal regulatory protein a (SIRPα(CD172a)) and CD40 Ligand (CD40L), OX40 Ligand (OX40L) or LIGHT exhibit synergistic effects in treating cancer when administered in combinations with certain specific anti-cancer agents. In addition, these agents potentiate the phagocytosis-stimulating activity of the SIRPα-based chimeric proteins disclosed herein. Furthermore, these agents cause the induction of CD47 and/or pro-phagocytic signals. The specific anti-cancer agents that cause these effect include a hypomethylating agent/epigenetic regulators such as azacitidine, a proteasomal inhibitor such as bortezomib, an anti-metabolites such as cytarabine (ARA-C) or 5-fluorouacil (5-FU), a DNA synthesis inhibitors such as cytarabine (ARA-C) or 5-fluorouacil (5-FU), an immune checkpoint inhibitors such as an anti-PD-L1 antibody, an anthracycline such as doxorubicin, a topoisomerase II inhibitor such as doxorubicin, an innate immune checkpoint inhibitors such as anti-CD47, a Bcl2 inhibitors such as venetoclax, a protein neddylation inhibitors such as pevonedistat, a microtubule-targeting agent such as paclitaxel, a thymidylate synthase (TS) inhibitor such as 5-fluorouracil, a platinum drug such as cisplatin or oxaliplatin, a topoisomerase I inhibitors such as irinotecan, an anti-BCMA antibody, an anti-CD38 antibody such as daratumumab, a Immunomodulatory Imide Drug (IMiD) such as pomalidamide or lenolidamide, an anti-SLAMF7 antibody such as elotuzumab, an anti-CD123 antibody, and a reactivator of mutated p53 such as APR-246, anti-FOLR1 antibody, or a combination thereof.

Accordingly, in one aspect, the present disclosure provides a method for treating a cancer in a subject in need thereof comprising: administering to the subject a first pharmaceutical composition comprising a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain; and administering to the subject a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof.

In one aspect, the present disclosure provides a method for treating a cancer in a subject comprising: administering to the subject a pharmaceutical composition comprising a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain; wherein the subject has undergone or is undergoing treatment with a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof.

In one aspect, the present disclosure provides a method for treating a cancer in a subject comprising: administering to the subject a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; wherein the subject has undergone or is undergoing treatment with a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain.

Importantly, since the chimeric proteins used in methods of the present disclosure disrupt, block, reduces, inhibit, and/or sequester the transmission of immune inhibitory signals, e.g., originating from a cancer cell that is attempting to avoid its detection and/or destruction and/or enhance, increase, and/or stimulate the transmission of an immune stimulatory signal to an anti-cancer immune cell, the methods can provide an anti-tumor effect by multiple distinct pathways. By treating cancer via multiple distinct pathways, the methods of the present disclosure are more likely to provide any anti-tumor effect in a patient and/or to provide an enhanced anti-tumor effect in a patient. Moreover, since the methods operate by multiple distinct pathways, they can be efficacious, at least, in patients who do not respond, respond poorly, or become resistant to treatments that target one of the pathways. Thus, a patient who is a poor responder to treatments acting via one of the two pathways, can receive a therapeutic benefit by targeting multiple pathways.

Without wishing to be bound by theory, the SIRPα(CD172a)-Fc-CD40L chimeric proteins of the present disclosure and/or the SIRPα(CD172a)-Fc-CD40L chimeric proteins used in methods of the present disclosure may operate according to the following mechanisms. First, the SIRPα(CD172a)-Fc-CD40L chimeric proteins may directly activate antigen presenting cells by binding to CD40 on APCs. Here, an advantage may be antigen-specific CD8 stimulation and/or programming of immune memory. When used in a combination, antibodies related to checkpoint molecules may increase CD40 target density for SIRPα(CD172a)-Fc-CD40L costimuation and upregulation of antigen presentation machinery. Second, the SIRPα(CD172a)-Fc-CD40L chimeric proteins may directly block CD47 inhibition by tumor cells blocking and sequestering CD47 on tumor cells. Here, an advantage may be enhanced tumor phagocytosis and increased antigen cross-presentation. When used in a combination, antibody-dependent cellular cytotoxicity-related antibodies increase targeted tumor phagocytosis, antigen cross-presentation and anti-tumor response.

In embodiments, the chimeric proteins of the present disclosure and/or chimeric proteins used in methods of the present disclosure eliminate or reduce side effects associated with disrupting the SIRP1α/CD47 signaling axis. In embodiments, the present chimeric proteins or methods utilizing the same eliminate or reduce hematological adverse effects. In embodiments, the present chimeric proteins or methods utilizing the same eliminate or reduce the extent of reductions in the number of circulating red blood cells and platelets, hemolysis, hemagglutination, thrombocytopenia, and/or anemia. In embodiments, the present chimeric proteins or methods utilizing the same demonstrate comparatively less hematological adverse effects than an anti-CD47 antibody.

### The First Pharmaceutical Composition

The methods of the present disclosure comprise methods for treating cancer, which, in embodiments, comprise administering a pharmaceutical composition comprising a chimeric protein capable of blocking immune inhibitory signals and/or stimulating immune activating signals.

In one aspect, the present disclosure relates to a method for treating a cancer in a subject in need thereof comprising: a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain.

Transmembrane proteins typically consist of an extracellular domain, one or a series of transmembrane domains, and an intracellular domain. Without wishing to be bound by theory, the extracellular domain of a transmembrane protein is responsible for interacting with a soluble receptor or ligand or membrane-bound receptor or ligand (i.e., a membrane of an adjacent cell) in the extracellular environment. Without wishing to be bound by theory, the trans-membrane domain(s) is responsible for localizing the transmembrane protein to the plasma membrane. Without wishing to be bound by theory, the intracellular domain of a transmembrane protein is responsible for coordinating interactions with cellular signaling molecules to coordinate intracellular responses with the extracellular environment (or visa-versa).

In embodiments, the chimeric proteins useful in the methods disclosed herein eliminate or reduce side effects associated with disrupting the SIRP1α/CD47 signaling axis. In embodiments, the present chimeric proteins or methods utilizing the same eliminate or reduce hematological adverse effects. In embodiments, the present chimeric proteins or methods utilizing the same eliminate or reduce the extent of reductions in the number of circulating red blood cells and platelets, hemolysis, hemagglutination, thrombocytopenia, and/or anemia. In embodiments, the present chimeric proteins or methods utilizing the same demonstrate comparatively less hematological adverse effects than an anti-CD47 antibody.

In embodiments, an extracellular domain refers to a portion of a transmembrane protein which is sufficient for binding to a ligand or receptor and is effective in transmitting a signal to a cell. In embodiments, an extracellular domain is the entire amino acid sequence of a transmembrane protein which is normally present at the exterior of a cell or of the cell membrane. In embodiments, an extracellular domain is that portion of an amino acid sequence of a transmembrane protein which is external of a cell or of the cell membrane and is needed for signal transduction and/or ligand binding as may be assayed using methods know in the art (e.g., in vitro ligand binding and/or cellular activation assays).

In embodiments, an extracellular domain refers to a portion of a transmembrane protein which is sufficient for binding to a ligand or receptor and is effective in transmitting a signal to a cell. In embodiments, an extracellular domain is the entire amino acid sequence of a transmembrane protein which is normally present at the exterior of a cell or of the cell membrane. In embodiments, an extracellular domain is that portion of an amino acid sequence of a transmembrane protein which is external of a cell or of the cell membrane and is needed for signal transduction and/or ligand binding as may be assayed using methods know in the art (e.g., in vitro ligand binding and/or cellular activation assays).

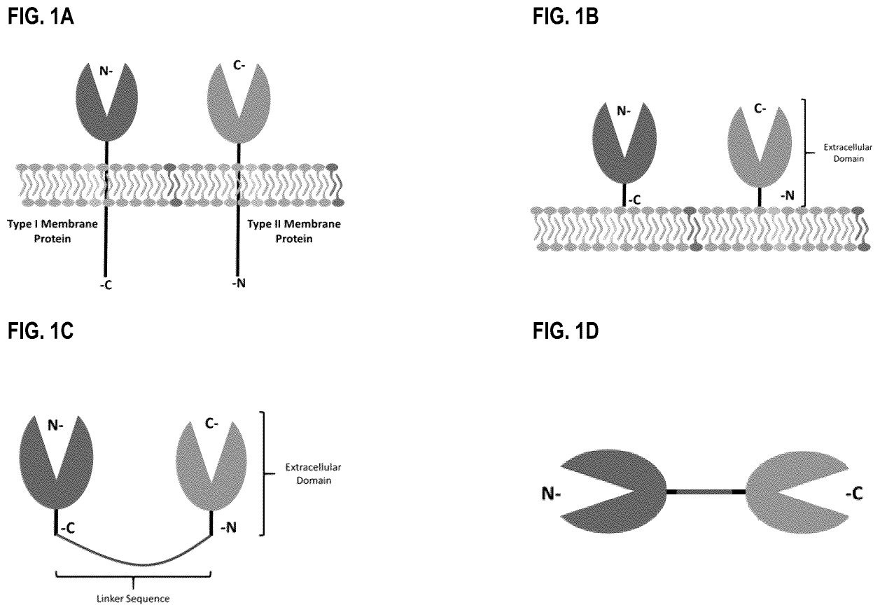

There are generally two types of single-pass transmembrane proteins: Type I transmembrane proteins which have an extracellular amino terminus and an intracellular carboxy terminus (see, FIG. 1A, left protein) and Type II transmembrane proteins which have an extracellular carboxy terminus and an intracellular amino terminus (see, FIG. 1A, right protein). Type I and Type II transmembrane proteins can be either receptors or ligands. For Type I transmembrane proteins (e.g., SIRPα(CD172a)), the amino terminus of the protein faces outside the cell, and therefore contains the functional domains that are responsible for interacting with other binding partners (either ligands or receptors) in the extracellular environment (see, FIG. 1B, left protein). For Type II transmembrane proteins (e.g., CD40L OX40L, and LIGHT), the carboxy terminus of the protein faces outside the cell, and therefore contains the functional domains that are responsible for interacting with other binding partners (either ligands or receptors) in the extracellular environment (see, FIG. 1B, right protein). Thus, these two types of transmembrane proteins have opposite orientations to each other relative to the cell membrane.

Chimeric proteins used in methods of the present disclosure comprise an extracellular domain of a Type I transmembrane protein, e.g., SIRPα(CD172a), and an extracellular domain of a Type II transmembrane protein selected from CD40L, OX40L, and LIGHT. Thus, a chimeric protein used in a method of the present disclosure comprises, at least, a first domain comprising the extracellular domain of SIRPα(CD172a), which is connected—directly or via a linker—to a second domain comprising the extracellular domain of CD40L, OX40L, or LIGHT. As illustrated in FIG. 1C and FIG. 1D, when the domains are linked in an amino-terminal to carboxy-terminal orientation, the first domain is located on the “left” side of the chimeric protein and is “outward facing” and the second domain is located on “right” side of the chimeric protein and is “outward facing”.

Other configurations of first and second domains are envisioned, e.g., the first domain is inward facing and the second domain is outward facing, the first domain is outward facing and the second domain is inward facing, and the first and second domains are both inward facing. When both domains are “inward facing”, the chimeric protein would have an amino-terminal to carboxy-terminal configuration comprising an extracellular domain of a Type II transmembrane protein, a linker, and an extracellular domain of Type I transmembrane protein. In such configurations, it may be necessary for the chimeric protein to include extra “slack”, as described elsewhere herein, to permit binding domains of the chimeric protein to one or both of its receptors/ligands.

In embodiments, the heterologous chimeric protein comprises: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L ligand, and (c) a linker linking the first domain and the second domain.

In embodiments, a heterologous chimeric protein comprises a first domain which comprises substantially the entire extracellular domain of SIRPα(CD172a), and/or the second domain which comprises substantially the entire extracellular domain of CD40L. In embodiments, the first domain which comprises substantially the entire extracellular domain of SIRPα(CD172a). In embodiments, the second domain which comprises substantially the entire extracellular domain of CD40L.

In embodiments, the heterologous chimeric protein comprises: (a) a first domain comprising a portion of the extracellular domain of SIRPα(CD172a), wherein the portion is capable of binding a SIRPα(CD172a) ligand, (b) a second domain comprising a portion of the extracellular domain of OX40L, wherein the portion is capable of binding an OX40L ligand, and (c) a linker linking the first domain and the second domain.

In embodiments, a heterologous chimeric protein comprises a first domain which comprises substantially the entire extracellular domain of SIRPα(CD172a), and/or the second domain which comprises substantially the entire extracellular domain of OX40L. In embodiments, the first domain which comprises substantially the entire extracellular domain of SIRPα(CD172a). In embodiments, the second domain which comprises substantially the entire extracellular domain of OX40L.

In embodiments, the heterologous chimeric protein comprises: (a) a first domain comprising a portion of the extracellular domain of SIRPα(CD172a), wherein the portion is capable of binding a SIRPα(CD172a) ligand, (b) a second domain comprising a portion of the extracellular domain of LIGHT, wherein the portion is capable of binding an LIGHT ligand, and (c) a linker linking the first domain and the second domain.

In embodiments, a heterologous chimeric protein comprises a first domain which comprises substantially the entire extracellular domain of SIRPα(CD172a), and/or the second domain which comprises substantially the entire extracellular domain of LIGHT. In embodiments, the first domain which comprises substantially the entire extracellular domain of SIRPα(CD172a). In embodiments, the second domain which comprises substantially the entire extracellular domain of LIGHT.

The First Domain In embodiments, the first domain comprises a portion of Signal regulatory protein a (SIRPα). In embodiments, the first domain comprises the extracellular domain of SIRPα. In embodiments, the first domain comprises the CD47-binding portion of SIRPα.

In embodiments, a chimeric protein used in methods of the present disclosure comprises the extracellular domain of human SIRPα(CD172a) which comprises the following amino acid sequence:

(SEQ ID NO: 57)
EEELQVIQPDKSVLVAAGETATLRCTATSLIPVGPIQWFRGAGPGRELI
YNQKEGHFPRVTTVSDLTKRNNMDFSIRIGNITPADAGTYYCVKFRKGS
PDDVEFKSGAGTELSVRAKPSAPVVSGPAARATPQHTVSFTCESHGFSP
RDITLKWFKNGNELSDFQTNVDPVGESVSYSIHSTAKVVLTREDVHSQV
ICEVAHVTLQGDPLRGTANLSETIRVPPTLEVTQQPVRAENQVNVTCQV
RKFYPQRLQLTWLENGNVSRTETASTVTENKDGTYNWMSWLLVNVSAHR
DDVKLTCQVEHDGQPAVSKSHDLKVSAHPKEQGSNTAAENTGSNERNI
Y.

In embodiments, a chimeric protein used in methods of the present disclosure comprises a variant of the extracellular domain of SIRPα(CD172a). As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 57.

In embodiments, the variant of the extracellular domain of SIRPα(CD172a) has at least about 95% sequence identity with SEQ ID NO: 57 One of ordinary skill may select variants of the known amino acid sequence of SIRPα(CD172a) by consulting the literature, e.g. LEE, et al., “Novel Structural Determinants of SIRPα that Mediate Binding of CD47,” The Journal of Immunology, 179, 7741-7750, 2007 and HATHERLEY, et al., “The Structure of the Macrophage Signal Regulatory Protein a (SIRPα) Inhibitory Receptor Reveals a Binding Face Reminiscent of That Used by T Cell Receptors,” The Journal Of Biological Chemistry, Vol. 282, No. 19, pp. 14567-14575, 2007, each of which is incorporated by reference in its entirety.

### The Second Domain

In embodiments, a chimeric protein used in methods of the present disclosure comprises the extracellular domain of human CD40L which comprises the following amino acid sequence:

(SEQ ID NO: 58)
HRRLDKIEDERNLHEDFVFMKTIQRCNTGERSLSLLNCEEIKSQFEGFV
KDIMLNKEETKKENSFEMQKGDQNPQIAAHVISEASSKTTSVLQWAEKG
YYTMSNNLVTLENGKQLTVKRQGLYYIYAQVTFCSNREASSQAPFIASL
CLKSPGRFERILLRAANTHSSAKPCGQQSIHLGGVFELQPGASVFVNVT
DPSQVSHGTGFTSFGLLKL.

In embodiments, a chimeric protein used in methods of the present disclosure comprises a variant of the extracellular domain of CD40L. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the variant of the extracellular domain of CD40L has at least about 95% sequence identity with SEQ ID NO: 58 One of ordinary skill may select variants of the known amino acid sequence of CD40L by consulting the literature, e.g. An, et al. “Crystallographic and Mutational Analysis of the CD40-CD154 Complex and Its Implications for Receptor Activation”, The Journal of Biological Chemistry 286, 11226-11235, which is incorporated by reference in its entirety.

In embodiments, a chimeric protein used in methods of the present disclosure comprises the extracellular domain of human OX40L which comprises the following amino acid sequence:

(SEQ ID NO: 59)
QVSHRYPRIQSIKVQFTEYKKEKGFILTSQKEDEIMKVQNNSVIINCDG
FYLISLKGYFSQEVNISLHYQKDEEPLFQLKKVRSVNSLMVASLTYKDK
VYLNVTTDNTSLDDFHVNGGELILIHQNPGEFCVL.

In embodiments, a chimeric protein comprises a variant of the extracellular domain of OX40L. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 59.

In embodiments, the variant of the extracellular domain of OX40L has at least about 95% sequence identity with SEQ ID NO: 59

One of ordinary skill may select variants of the known amino acid sequence of OX40L by consulting the literature, e.g., CROFT, et al., “The Significance of OX40 and OX40L to T cell Biology and Immune Disease,” Immunol Rev., 229(1), PP. 173-191, 2009 and BAUM, et al., “Molecular characterization of murine and human OX40/OX40 ligand systems: identification of a human OX40 ligand as the HTL V-1-regulated protein gp34,” The EMBO Journal, Vol. 13, No. 77, PP. 3992-4001, 1994, each of which is incorporated by reference in its entirety.

In embodiments, a chimeric protein used in methods of the present disclosure comprises the extracellular domain of human LIGHT which comprises the following amino acid sequence:

(SEQ ID NO: 62)
LQLHWRLGEMVTRLPDGPAGSWEQLIQERRSHEVNPAAHLTGANSSLTG
SGGPLLWETQLGLAFLRGLSYHDGALVVTKAGYYYIYSKVQLGGVGCPL
GLASTITHGLYKRTPRYPEELELLVSQQSPCGRATSSSRVWWDSSFLGG
VVHLEAGEKVVVRVLDERLVRLRDGTRSYFGAFMV.

In embodiments, a chimeric protein comprises a variant of the extracellular domain of LIGHT. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 62.

In embodiments, the variant of the extracellular domain of LIGHT has at least about 95% sequence identity with SEQ ID NO: 62

One of ordinary skill may select variants of the known amino acid sequence of LIGHT by consulting the literature, e.g., Mauri, et al., “LIGHT, a new member of the TNF superfamily, and lymphotoxin alpha are ligands for herpesvirus entry mediator.” Immunity 8 (1), 21-30 (1998); Tamada et al., “LIGHT, a TNF-like molecule, costimulates T cell proliferation and is required for dendritic cell-mediated allogeneic T cell response.” J. Immunol. 164 (8), 4105-4110 (2000); Liu et al., “Mechanistic basis for functional promiscuity in the TNF and TNF receptor superfamilies: structure of the LIGHT:DcR3 assembly” Structure 22 1252-62 (2014); Faustman et al., “Structural principles of tumor necrosis factor superfamily signaling.” Sci Signal 11 (2018); Sudhamsu et al., “Dimerization of LTRR by LTα1β2 is necessary and sufficient for signal transduction” Proc. Natl. Acad. Sci. U.S.A. 110 19896-19901 (2013); Savvides et al., “Mechanisms of immunomodulation by mammalian and viral decoy receptors: insights from structures. Felix J, SN. Nat Rev Immunol 17 112-129 (2017)”; Ward-Kavanagh et al., “The TNF Receptor Superfamily in Co-stimulating and Co-inhibitory Responses.” Immunity 44 1005-1019 (2016); and Wajant “Principles of antibody-mediated TNF receptor activation.” Cell Death Differ 22 1727-1741 (2015), each of which is incorporated by reference in its entirety.

In any herein-disclosed aspect and embodiment, the chimeric protein may comprise an amino acid sequence having one or more amino acid mutations relative to any of the protein sequences disclosed herein. In embodiments, the one or more amino acid mutations may be independently selected from substitutions, insertions, deletions, and truncations.

In embodiments, the amino acid mutations are amino acid substitutions, and may include conservative and/or non-conservative substitutions. “Conservative substitutions” may be made, for instance, based on similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe. As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. As used herein, “non-conservative substitutions” are defined as exchanges of an amino acid by another amino acid listed in a different group of the six standard amino acid groups (1) to (6) shown above.

In embodiments, the substitutions may also include non-classical amino acids (e.g., selenocysteine, pyrrolysine, N-formylmethionine β-alanine, GABA and 6-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ϵ-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as § methyl amino acids, C α-methyl amino acids, N α-methyl amino acids, and amino acid analogs in general).

Mutations may also be made to the nucleotide sequences of the chimeric proteins by reference to the genetic code, including taking into account codon degeneracy.

In embodiments, a chimeric protein is capable of binding murine ligand(s)/receptor(s).

In embodiments, a chimeric protein is capable of binding human ligand(s)/receptor(s).

In embodiments, each extracellular domain (or variant thereof) of the chimeric protein binds to its cognate receptor or ligand with a K_{D} of about 1 nM to about 5 nM, for example, about 1 nM, about 1.5 nM, about 2 nM, about 2.5 nM, about 3 nM, about 3.5 nM, about 4 nM, about 4.5 nM, or about 5 nM. In embodiments, the chimeric protein binds to a cognate receptor or ligand with a K_{D} of about 5 nM to about 15 nM, for example, about 5 nM, about 5.5 nM, about 6 nM, about 6.5 nM, about 7 nM, about 7.5 nM, about 8 nM, about 8.5 nM, about 9 nM, about 9.5 nM, about 10 nM, about 10.5 nM, about 11 nM, about 11.5 nM, about 12 nM, about 12.5 nM, about 13 nM, about 13.5 nM, about 14 nM, about 14.5 nM, or about 15 nM.

In embodiments, each extracellular domain (or variant thereof) of the chimeric protein binds to its cognate receptor or ligand with a K_{D} of less than about 1 pM, about 900 nM, about 800 nM, about 700 nM, about 600 nM, about 500 nM, about 400 nM, about 300 nM, about 200 nM, about 150 nM, about 130 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human CD47 and/or CD40 with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry).

As used herein, a variant of an extracellular domain is capable of binding the receptor/ligand of a native extracellular domain. For example, a variant may include one or more mutations in an extracellular domain which do not affect its binding affinity to its receptor/ligand; alternately, the one or more mutations in an extracellular domain may improve binding affinity for the receptor/ligand; or the one or more mutations in an extracellular domain may reduce binding affinity for the receptor/ligand, yet not eliminate binding altogether. In embodiments, the one or more mutations are located outside the binding pocket where the extracellular domain interacts with its receptor/ligand. In embodiments, the one or more mutations are located inside the binding pocket where the extracellular domain interacts with its receptor/ligand, as long as the mutations do not eliminate binding altogether. Based on the skilled artisan's knowledge and the knowledge in the art regarding receptor-ligand binding, s/he would know which mutations would permit binding and which would eliminate binding.

In embodiments, the chimeric protein exhibits enhanced stability, high-avidity binding characteristics, prolonged off-rate for target binding and protein half-life relative to single-domain fusion protein or antibody controls.

A chimeric protein used in a method of the present disclosure may comprise more than two extracellular domains. For example, the chimeric protein may comprise three, four, five, six, seven, eight, nine, ten, or more extracellular domains. A second extracellular domain may be separated from a third extracellular domain via a linker, as disclosed herein. Alternately, a second extracellular domain may be directly linked (e.g., via a peptide bond) to a third extracellular domain. In embodiments, a chimeric protein includes extracellular domains that are directly linked and extracellular domains that are indirectly linked via a linker, as disclosed herein.

Chimeric proteins of the present disclosure and/or chimeric proteins used in methods of the present disclosure have a first domain which is sterically capable of binding its ligand/receptor and/or a second domain which is sterically capable of binding its ligand/receptor. This means that there is sufficient overall flexibility in the chimeric protein and/or physical distance between an extracellular domain (or a portion thereof) and the rest of the chimeric protein such that the ligand/receptor binding domain of the extracellular domain is not sterically hindered from binding its ligand/receptor. This flexibility and/or physical distance (which is herein referred to as “slack”) may be normally present in the extracellular domain(s), normally present in the linker, and/or normally present in the chimeric protein (as a whole). Alternately, or additionally, the chimeric protein may be modified by including one or more additional amino acid sequences (e.g., the joining linkers described below) or synthetic linkers (e.g., a polyethylene glycol (PEG) linker) which provide additional slack needed to avoid steric hindrance.

### The Linker

In embodiments, the chimeric protein used in a method of the present disclosure comprises a linker.

In embodiments, the linker comprising at least one cysteine residue capable of forming a disulfide bond. The at least one cysteine residue is capable of forming a disulfide bond between a pair (or more) of chimeric proteins. Without wishing to be bound by theory, such disulfide bond forming is responsible for maintaining a useful multimeric state of chimeric proteins. This allows for efficient production of the chimeric proteins; it allows for desired activity in vitro and in vivo.

Importantly, inter alia, stabilization in a linker region including one or more disulfide bonds provides for improved chimeric proteins that can maintain a stable and producible multimeric state.

In a chimeric protein used in a method of the present disclosure, the linker is a polypeptide selected from a flexible amino acid sequence, an IgG hinge region, or an antibody sequence.

In embodiments, the linker is derived from naturally-occurring multi-domain proteins or is an empirical linker as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the linker comprises a polypeptide. In embodiments, the polypeptide is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In embodiments, the linker is flexible.

In embodiments, the linker is rigid.

In embodiments, the linker is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines).

In embodiments, the linker comprises a hinge region of an antibody (e.g., of IgG, IgA, IgD, and IgE, inclusive of subclasses (e.g., IgG1, IgG2, IgG3, and IgG4, and IgA1, and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of C_{H1} to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in C_{H2}. Id. The core hinge region of wild-type human IgG1 contains the sequence CPPC (SEQ ID NO: 24) which, when dimerized by disulfide bond formation, results in a cyclic octapeptide believed to act as a pivot, thus conferring flexibility. In embodiments, the present linker comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In embodiments, the linker of the present disclosure comprises one or more glycosylation sites.

In embodiments, the linker comprises an Fc domain of an antibody (e.g., of IgG, IgA, IgD, and IgE, inclusive of subclasses (e.g., IgG1, IgG2, IgG3, and IgG4, and IgA1 and IgA2)).

In a chimeric protein used in a method of the present disclosure, the linker comprises a hinge-CH2-CH3 Fc domain derived from IgG4. In embodiments, the linker comprises a hinge-CH2-CH3 Fc domain derived from a human IgG4. In embodiments, the linker comprises an amino acid sequence that is at least 95% identical to the amino acid sequence of any one of SEQ ID NO: 1 to SEQ ID NO: 3, e.g., at least 95% identical to the amino acid sequence of SEQ ID NO: 2. In embodiments, the linker comprises one or more joining linkers, such joining linkers independently selected from SEQ ID NO: 4 to SEQ ID NO: 50 (or a variant thereof). In embodiments, the linker comprises two or more joining linkers each joining linker independently selected from SEQ ID NO: 4 to SEQ ID NO: 50 (or a variant thereof); wherein one joining linker is N terminal to the hinge-CH2-CH3 Fc domain and another joining linker is C terminal to the hinge-CH2-CH3 Fc domain.

In embodiments, the linker comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the chimeric proteins used in methods of the present disclosure.

In embodiments, the Fc domain in a linker contains one or more amino acid substitutions at amino acid residue 250, 252, 254, 256, 308, 309, 311, 416, 428, 433 or 434 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference), or equivalents thereof. In embodiments, the amino acid substitution at amino acid residue 250 is a substitution with glutamine. In embodiments, the amino acid substitution at amino acid residue 252 is a substitution with tyrosine, phenylalanine, tryptophan or threonine. In embodiments, the amino acid substitution at amino acid residue 254 is a substitution with threonine. In embodiments, the amino acid substitution at amino acid residue 256 is a substitution with serine, arginine, glutamine, glutamic acid, aspartic acid, or threonine. In embodiments, the amino acid substitution at amino acid residue 308 is a substitution with threonine. In embodiments, the amino acid substitution at amino acid residue 309 is a substitution with proline. In embodiments, the amino acid substitution at amino acid residue 311 is a substitution with serine. In embodiments, the amino acid substitution at amino acid residue 385 is a substitution with arginine, aspartic acid, serine, threonine, histidine, lysine, alanine or glycine. In embodiments, the amino acid substitution at amino acid residue 386 is a substitution with threonine, proline, aspartic acid, serine, lysine, arginine, isoleucine, or methionine. In embodiments, the amino acid substitution at amino acid residue 387 is a substitution with arginine, proline, histidine, serine, threonine, or alanine. In embodiments, the amino acid substitution at amino acid residue 389 is a substitution with proline, serine or asparagine. In embodiments, the amino acid substitution at amino acid residue 416 is a substitution with serine. In embodiments, the amino acid substitution at amino acid residue 428 is a substitution with leucine. In embodiments, the amino acid substitution at amino acid residue 433 is a substitution with arginine, serine, isoleucine, proline, or glutamine. In embodiments, the amino acid substitution at amino acid residue 434 is a substitution with histidine, phenylalanine, or tyrosine.

In embodiments, the Fc domain linker (e.g., comprising an IgG constant region) comprises one or more mutations such as substitutions at amino acid residue 252, 254, 256, 433, 434, or 436 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). In embodiments, the IgG constant region includes a triple M252Y/S254T/T256E mutation or YTE mutation. In embodiments, the IgG constant region includes a triple H433K/N434F/Y436H mutation or KFH mutation. In embodiments, the IgG constant region includes an YTE and KFH mutation in combination.

In embodiments, the linker comprises an IgG constant region that contains one or more mutations at amino acid residues 250, 253, 307, 310, 380, 428, 433, 434, and 435 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). Illustrative mutations include T250Q, M428L, T307A, E380A, 1253A, H310A, M428L, H433K, N434A, N434F, N434S, and H435A. In embodiments, the IgG constant region comprises a M428L/N434S mutation or LS mutation. In embodiments, the IgG constant region comprises a T250Q/M428L mutation or QL mutation. In embodiments, the IgG constant region comprises an N434A mutation. In embodiments, the IgG constant region comprises a T307A/E380A/N434A mutation or AAA mutation. In embodiments, the IgG constant region comprises an 1253A/H310A/H435A mutation or IHH mutation. In embodiments, the IgG constant region comprises a H433K/N434F mutation. In embodiments, the IgG constant region comprises a M252Y/S254T/T256E and a H433K/N434F mutation in combination.

Additional exemplary mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al. Nature (2014) 514:642-645, Grevys et al. Journal of Immunology. (2015), 194(11):5497-508, and U.S. Pat. No. 7,083,784, the entire contents of which are hereby incorporated by reference.

An illustrative Fc stabilizing mutant is S228P. Illustrative Fc half-life extending mutants are T250Q, M428L, V308T, L309P, and Q311S and the present linkers may comprise 1, or 2, or 3, or 4, or 5 of these mutants.

In embodiments, the chimeric protein binds to FcRn with high affinity. In embodiments, the chimeric protein may bind to FcRn with a K_{D} of about 1 nM to about 80 nM. For example, the chimeric protein may bind to FcRn with a K_{D} of about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 71 nM, about 72 nM, about 73 nM, about 74 nM, about 75 nM, about 76 nM, about 77 nM, about 78 nM, about 79 nM, or about 80 nM. In embodiments, the chimeric protein may bind to FcRn with a K_{D} of about 9 nM. In embodiments, the chimeric protein does not substantially bind to other Fc receptors (i.e. other than FcRn) with effector function.

In embodiments, the Fc domain in a linker has the amino acid sequence of SEQ ID NO: 1 (see Table 1, below), or at least 90%, or 93%, or 95%, or 97%, or 98%, or 99% identity thereto. In embodiments, mutations are made to SEQ ID NO: 1 to increase stability and/or half-life. For instance, in embodiments, the Fc domain in a linker comprises the amino acid sequence of SEQ ID NO: 2 (see Table 1, below), or at least 90%, or 93%, or 95%, or 97%, or 98%, or 99% identity thereto. For instance, in embodiments, the Fc domain in a linker comprises the amino acid sequence of SEQ ID NO: 3 (see Table 1, below), or at least 90%, or 93%, or 95%, or 97%, or 98%, or 99% identity thereto.

Further, one or more joining linkers may be employed to connect an Fc domain in a linker (e.g., one of SEQ ID NO: 1, SEQ ID NO: 2, SEQ ID NO: 3 or at least 90%, or 93%, or 95%, or 97%, or 98%, or 99% identity thereto) and the extracellular domains. For example, any one of SEQ ID NO: 4, SEQ ID NO: 5, SEQ ID NO: 6, SEQ ID NO: 7, SEQ ID NO: 8, SEQ ID NO: 9, or variants thereof may connect an extracellular domain as disclosed herein and an Fc domain in a linker as disclosed herein. Optionally, any one of SEQ ID NO: 4 to SEQ ID NO: 50, or variants thereof are located between an extracellular domain as disclosed herein and an Fc domain as disclosed herein.

In embodiments, the chimeric proteins used in methods of the present disclosure may comprise variants of the joining linkers disclosed in Table 1, below. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NO: 4 to SEQ ID NO: 50.

In embodiments, the first and second joining linkers may be different or they may be the same.

Without wishing to be bound by theory, including a linker comprising at least a part of an Fc domain in a chimeric protein, helps avoid formation of insoluble and, likely, non-functional protein concatenated oligomers and/or aggregates. This is in part due to the presence of cysteines in the Fc domain which are capable of forming disulfide bonds between chimeric proteins.

In embodiments, a chimeric protein may comprise one or more joining linkers, as disclosed herein, and lack an Fc domain linker, as disclosed herein.

In embodiments, the first and/or second joining linkers are independently selected from the amino acid sequences of SEQ ID NO: 4 to SEQ ID NO: 50 and are provided in Table 1 below:

TABLE 1
Illustrative linkers (Fc domain linkers and joining linkers)

| SEQ | |
|---|---|
| ID | |
| NO. | Sequence |
| 1 | APEFLGGPSVFLFPPKPKDTLMISRTPEVTCVVVDVSQEDPEVQFNWYVDGVEVHNAKTKPR |
| | EEQFNSTYRVVSVLTVLHQDWLSGKEYKCKVSSKGLPSSIEKTISNATGQPREPQVYTLPPS |
| | QEEMTKNQVSLTCLVKGFYPSDIAVEWESNGQPENNYKTTPPVLDSDGSFFLYSRLTVDKSS |
| | WQEGNVFSCSVMHEALHNHYTQKSLSLSLGK |
| 2 | APEFLGGPSVFLFPPKPKDQLMISRTPEVTCVVVDVSQEDPEVQFNWYVDGVEVHNAKTKP |
| | REEQFNSTYRVVSVLTTPHSDWLSGKEYKCKVSSKGLPSSIEKTISNATGQPREPQVYTLPP |
| | SQEEMTKNQVSLTCLVKGFYPSDIAVEWESNGQPENNYKTTPPVLDSDGSFFLYSRLTVDKS |
| | SWQEGNVFSCSVLHEALHNHYTQKSLSLSLGK |
| 3 | APEFLGGPSVFLFPPKPKDQLMISRTPEVTCVVVDVSQEDPEVQFNWYVDGVEVHNAKTKP |
| | REEQFNSTYRVVSVLTVLHQDWLSGKEYKCKVSSKGLPSSIEKTISNATGQPREPQVYTLPP |
| | SQEEMTKNQVSLTCLVKGFYPSDIAVEWESNGQPENNYKTTPPVLDSDGSFFLYSRLTVDKS |
| | RWQEGNVFSCSVLHEALHNHYTQKSLSLSLGK |
| 4 | SKYGPPCPSCP |
| 5 | SKYGPPCPPCP |
| 6 | SKYGPP |
| 7 | IEGRMD |
| 8 | GGGVPRDCG |
| 9 | IEGRMDGGGGAGGGG |
| 10 | GGGSGGGS |
| 11 | GGGSGGGGSGGG |
| 12 | EGKSSGSGSESKST |
| 13 | GGSG |
| 14 | GGSGGGSGGGSG |
| 15 | EAAAKEAAAKEAAAK |
| 16 | EAAAREAAAREAAAREAAAR |
| 17 | GGGGSGGGGSGGGGSAS |
| 18 | GGGGAGGGG |
| 19 | GS or GGS or LE |
| 20 | GSGSGS |
| 21 | GSGSGSGSGS |
| 22 | GGGGSAS |
| 23 | APAPAPAPAPAPAPAPAPAP |
| 24 | CPPC |
| 25 | GGGGS |
| 26 | GGGGSGGGGS |
| 27 | GGGGSGGGGSGGGGS |
| 28 | GGGGSGGGGSGGGGSGGGGS |
| 29 | GGGGSGGGGSGGGGSGGGGSGGGGS |
| 30 | GGGGSGGGGSGGGGSGGGGSGGGGSGGGGS |
| 31 | GGGGSGGGGSGGGGSGGGGSGGGGSGGGGSGGGGS |
| 32 | GGGGSGGGGSGGGGSGGGGSGGGGSGGGGSGGGGSGGGGS |
| 33 | GGSGGSGGGGSGGGGS |
| 34 | GGGGGGGG |
| 35 | GGGGGG |
| 36 | EAAAK |
| 37 | EAAAKEAAAK |
| 38 | EAAAKEAAAKEAAAK |
| 39 | AEAAAKEAAAKA |
| 40 | AEAAAKEAAAKEAAAKA |
| 41 | AEAAAKEAAAKEAAAKEAAAKA |
| 42 | AEAAAKEAAAKEAAAKEAAAKEAAAKA |
| 43 | AEAAAKEAAAKEAAAKEAAAKALEAEAAAKEAAAKEAAAKEAAAKA |
| 44 | PAPAP |
| 45 | KESGSVSSEQLAQFRSLD |
| 46 | GSAGSAAGSGEF |
| 47 | GGGSE |
| 48 | GSESG |
| 49 | GSEGS |
| 50 | GEGGSGEGSSGEGSSSEGGGSEGGGSEGGGSEGGS |

In embodiments, the joining linker substantially comprises glycine and serine residues (e.g., about 30%, or about 40%, or about 50%, or about 60%, or about 70%, or about 80%, or about 90%, or about 95%, or about 97%, or about 98%, or about 99%, or about 100% glycines and serines). For example, in embodiments, the 5 joining linker is (Gly₄Ser)ₙ, where n is from about 1 to about 8, e.g., 1, 2, 3, 4, 5, 6, 7, or 8 (SEQ ID NO: 25 to SEQ ID NO: 32, respectively). In embodiments, the joining linker sequence is GGSGGSGGGGSGGGGS (SEQ ID NO: 33). Additional illustrative joining linkers include, but are not limited to, linkers having the sequence LE, (EAAAK)ₙ (n=1-3) (SEQ ID NO: 36 to SEQ ID NO: 38), A(EAAAK)nA (n=2-5) (SEQ ID NO: 39 to SEQ ID NO: 42), A(EAAAK)₄ALEA(EAAAK)₄A (SEQ ID NO: 43), PAPAP (SEQ ID NO: 44), KESGSVSSEQLAQFRSLD (SEQ ID NO: 45), GSAGSAAGSGEF (SEQ ID NO: 46), and (XP)ₙ, with X designating any amino acid, e.g., Ala, Lys, or Glu. In embodiments, the joining linker is GGS. In embodiments, a joining linker has the sequence (Gly)ₙ where n is any number from 1 to 100, for example: (Gly)₈ (SEQ ID NO: 34) and (Gly)₆ (SEQ ID NO: 35).

In embodiments, the joining linker is one or more of GGGSE (SEQ ID NO: 47), GSESG (SEQ ID NO: 48), GSEGS (SEQ ID NO: 49), GEGGSGEGSSGEGSSSEGGGSEGGGSEGGGSEGGS (SEQ ID NO: 50), and a joining linker of randomly placed G, S, and E every 4 amino acid intervals.

In embodiments, where a chimeric protein used in a method of the present disclosure comprises an extracellular domain (ECD) of a first transmembrane protein, one joining linker preceding an Fc domain, a second joining linker following the Fc domain, and an ECD of second transmembrane protein, the chimeric protein may comprise the following structure:
ECD-Joining Linker 1-Fc Domain-Joining Linker 2-ECD.

The combination of a first joining linker, an Fc Domain linker, and a second joining linker is referend to herein as a “modular linker”. In embodiments, a chimeric protein used in a method of the present disclosure comprises a modular linker as shown in Table 2:

TABLE 2
Illustrative modular linkers

| | | | Modular Linker = Joining |
|---|---|---|---|
| Joining | | Joining | Linker 1 + Fc + Joining |
| Linker 1 | Fc | Linker 2 | Linker 2 |
| SKYGPPCPSC | APEFLGGPSVFLFPPKPKDTL | IEGRMD | SKYGPPCPSCPAPEFLGGPSV |
| P | MISRTPEVTCVVVDVSQEDPE | (SEQ ID NO: | FLFPPKPKDTLMISRTPEVTCV |
| (SEQ ID NO: 4) | VQFNWYVDGVEVHNAKTKPR | 7) | VVDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVMHEALHNHYTQK | | LTVDKSSWQEGNVFSCSVMH |
| | SLSLSLGK (SEQ ID NO: 1) | | EALHNHYTQKSLSLSLGKIEGR |
| | | | MD (SEQ ID NO: 51) |
| SKYGPPCPSC | APEFLGGPSVFLFPPKPKDQL | IEGRMD | SKYGPPCPSCPAPEFLGGPSV |
| P | MISRTPEVTCVVVDVSQEDPE | (SEQ ID NO: | FLFPPKPKDQLMISRTPEVTCV |
| (SEQ ID NO: 4) | VQFNWYVDGVEVHNAKTKPR | 7) | VVDVSQEDPEVQFNWYVDGV |
| | EEQFNSTYRVVSVLTTPHSDW | | EVHNAKTKPREEQFNSTYRVV |
| | LSGKEYKCKVSSKGLPSSIEKT | | SVLTTPHSDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVLHEALHNHYTQKS | | LTVDKSSWQEGNVFSCSVLHE |
| | LSLSLGK (SEQ ID NO: 2) | | ALHNHYTQKSLSLSLGKIEGR |
| | | | MD (SEQ ID NO: 52) |
| SKYGPPCPSC | APEFLGGPSVFLFPPKPKDQL | IEGRMD | SKYGPPCPSCPAPEFLGGPSV |
| P | MISRTPEVTCVVVDVSQEDPE | (SEQ ID NO: | FLFPPKPKDQLMISRTPEVTCV |
| (SEQ ID NO: 4) | VQFNWYVDGVEVHNAKTKPR | 7) | VVDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSRWQE | | NNYKTTPPVLDSDGSFFLYSR |
| | GNVFSCSVLHEALHNHYTQKS | | LTVDKSRWQEGNVFSCSVLHE |
| | LSLSLGK (SEQ ID NO: 3) | | ALHNHYTQKSLSLSLGKIEGR |
| | | | MD (SEQ ID NO: 53) |
| SKYGPPCPPC | APEFLGGPSVFLFPPKPKDTL | IEGRMD | SKYGPPCPPCPAPEFLGGPSV |
| P | MISRTPEVTCVVVDVSQEDPE | (SEQ ID NO: | FLFPPKPKDTLMISRTPEVTCV |
| (SEQ ID NO: 5) | VQFNWYVDGVEVHNAKTKPR | 7 | VVDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVMHEALHNHYTQK | | LTVDKSSWQEGNVFSCSVMH |
| | SLSLSLGK (SEQ ID NO: 1) | | EALHNHYTQKSLSLSLGKIEGR |
| | | | MD (SEQ ID NO: 54) |
| SKYGPPCPPC | APEFLGGPSVFLFPPKPKDQL | IEGRMD | SKYGPPCPPCPAPEFLGGPSV |
| P | MISRTPEVTCVVVDVSQEDPE | (SEQ ID NO: | FLFPPKPKDQLMISRTPEVTCV |
| (SEQ ID NO: 5) | VQFNWYVDGVEVHNAKTKPR | 7) | VVDVSQEDPEVQFNWYVDGV |
| | EEQFNSTYRVVSVLTTPHSDW | | EVHNAKTKPREEQFNSTYRVV |
| | LSGKEYKCKVSSKGLPSSIEKT | | SVLTTPHSDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVLHEALHNHYTQKS | | LTVDKSSWQEGNVFSCSVLHE |
| | LSLSLGK (SEQ ID NO: 2) | | ALHNHYTQKSLSLSLGKIEGR |
| | | | MD (SEQ ID NO: 55) |
| SKYGPPCPPC | APEFLGGPSVFLFPPKPKDQL | IEGRMD | SKYGPPCPPCPAPEFLGGPSV |
| P | MISRTPEVTCVVVDVSQEDPE | (SEQ ID NO: | FLFPPKPKDQLMISRTPEVTCV |
| (SEQ ID NO: 5) | VQFNWYVDGVEVHNAKTKPR | 7) | VVDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSRWQE | | NNYKTTPPVLDSDGSFFLYSR |
| | GNVFSCSVLHEALHNHYTQKS | | LTVDKSRWQEGNVFSCSVLHE |
| | LSLSLGK (SEQ ID NO: 3) | | ALHNHYTQKSLSLSLGKIEGR |
| | | | MD (SEQ ID NO: 56) |

In embodiments, the chimeric proteins used in methods of the present disclosure may comprise variants of the modular linkers disclosed in Table 2, above. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NO: 51 to SEQ ID NO: 56.

In embodiments, the linker may be flexible, including without limitation highly flexible. In embodiments, the linker may be rigid, including without limitation a rigid alpha helix. Characteristics of illustrative joining linkers is shown below in Table 3:

TABLE 3
Characteristics of illustrative joining linkers

| Joining Linker Sequence | Characteristics |
|---|---|
| SKYGPPCPPCP (SEQ ID NO: 5) | IgG4 Hinge Region |
| IEGRMD (SEQ ID NO: 7) | Linker |
| GGGVPRDCG (SEQ ID NO: 8) | Flexible |
| GGGSGGGS (SEQ ID NO: 10) | Flexible |
| GGGSGGGGSGGG (SEQ ID NO: 11) | Flexible |
| EGKSSGSGSESKST (SEQ ID NO: 12) | Flexible + soluble |
| GGSG (SEQ ID NO: 13) | Flexible |
| GGSGGGSGGGSG (SEQ ID NO: 14) | Flexible |
| EAAAKEAAAKEAAAK (SEQ ID NO: 15) | Rigid Alpha Helix |
| EAAAREAAAREAAAREAAAR (SEQ ID NO: 16) | Rigid Alpha Helix |
| GGGGSGGGGSGGGGSAS (SEQ ID NO: 17) | Flexible |
| GGGGAGGGG (SEQ ID NO: 18) | Flexible |
| GS (SEQ ID NO: 19) | Highly flexible |
| GSGSGS (SEQ ID NO: 20) | Highly flexible |
| GSGSGSGSGS (SEQ ID NO: 21) | Highly flexible |
| GGGGSAS (SEQ ID NO: 22) | Flexible |
| APAPAPAPAPAPAPAPAPAP (SEQ ID NO: 23) | Rigid |

In embodiments, the linker may be functional. For example, without limitation, the linker may function to improve the folding and/or stability, improve the expression, improve the pharmacokinetics, and/or improve the bioactivity of the chimeric protein used in a method of the present disclosure. In another example, the linker may function to target the chimeric protein to a particular cell type or location.

In embodiments, a chimeric protein used in a method of the present disclosure comprises only one joining linkers.

In embodiments, a chimeric protein used in a method of the present disclosure lacks joining linkers.

In embodiments, the linker is a synthetic linker such as polyethylene glycol (PEG).

In embodiments, a chimeric protein has a first domain which is sterically capable of binding its ligand/receptor and/or the second domain which is sterically capable of binding its ligand/receptor. Thus, there is enough overall flexibility in the chimeric protein and/or physical distance between an extracellular domain (or portion thereof) and the rest of the chimeric protein such that the ligand/receptor binding domain of the extracellular domain is not sterically hindered from binding its ligand/receptor. This flexibility and/or physical distance (which is referred to as “slack”) may be normally present in the extracellular domain(s), normally present in the linker, and/or normally present in the chimeric protein (as a whole). Alternately, or additionally, an amino acid sequence (for example) may be added to one or more extracellular domains and/or to the linker to provide the slack needed to avoid steric hindrance. Any amino acid sequence that provides slack may be added. In embodiments, the added amino acid sequence comprises the sequence (Gly)ₙ where n is any number from 1 to 100. Additional examples of addable amino acid sequence include the joining linkers described in Table 1 and Table 3. In embodiments, a polyethylene glycol (PEG) linker may be added between an extracellular domain and a linker to provide the slack needed to avoid steric hindrance. Such PEG linkers are well known in the art.

In embodiments, a heterologous chimeric protein comprises a first domain comprising a portion of SIRPα(CD172a), a second domain comprising a portion of CD40L, and a linker. In embodiments, the linker is a polypeptide selected from a flexible amino acid sequence, an IgG hinge region, and an antibody sequence. In embodiments, the linker comprises at least one cysteine residue capable of forming a disulfide bond and/or comprises a hinge-CH2-CH3 Fc domain. In embodiments, the linker comprises a hinge-CH2-CH3 Fc domain, e.g., from an IgG1 or from IgG4, including human IgG1 or IgG4. In embodiments, the linker comprises an amino acid sequence that is at least 95% identical to the amino acid sequence of SEQ ID NO: 1, SEQ ID NO: 2, or SEQ ID NO: 3. Thus, in embodiments, when a heterologous chimeric protein used in a method of the present disclosure comprises the extracellular domain of SIRPα(CD172a) (or a variant thereof), a linker comprising a hinge-CH2-CH3 Fc domain, and the extracellular domain of CD40L (or a variant thereof), it may be referred to herein as “SIRPα(CD172a)-Fc-CD40L”.

In embodiments, a SIRPα(CD172a)-Fc-CD40L chimeric protein of the present disclosure and/or a chimeric protein used in methods of the present disclosure has the following amino acid sequence:

(SEQ ID NO: 60)
EEELQVIQPDKSVLVAAGETATLRCTATSLIPVGPIQWFRGAGPGRELI
YNQKEGHFPRVTTVSDLTKRNNMDFSIRIGNITPADAGTYYCVKFRKGS
PDDVEFKSGAGTELSVRAKPSAPVVSGPAARATPQHTVSFTCESHGFSP
RDITLKWFKNGNELSDFQTNVDPVGESVSYSIHSTAKVVLTREDVHSQV
ICEVAHVTLQGDPLRGTANLSETIRVPPTLEVTQQPVRAENQVNVTCQV
RKFYPQRLQLTWLENGNVSRTETASTVTENKDGTYNWMSWLLVNVSAHR
DDVKLTCQVEHDGQPAVSKSHDLKVSAHPKEQGSNTAAENTGSNERNIY
SKYGPPCPPCPAPEFLGGPSVFLFPPKPKDQLMISRTPEVTCVVVDVSQ
EDPEVQFNWYVDGVEVHNAKTKPREEQFNSTYRVVSVLTVLHQDWLSGK
EYKCKVSSKGLPSSIEKTISNATGQPREPQVYTLPPSQEEMTKNQVSLT
CLVKGFYPSDIAVEWESNGQPENNYKTTPPVLDSDGSFFLYSRLTVDKS
RWQEGNVFSCSVLHEALHNHYTQKSLSLSLGKIEGRMDHRRLDKIEDER
NLHEDFVFMKTIQRCNTGERSLSLLNCEEIKSQFEGFVKDIMLNKEETK
KENSFEMQKGDQNPQIAAHVISEASSKTTSVLQWAEKGYYTMSNNLVTL
ENGKQLTVKRQGLYYIYAQVTFCSNREASSQAPFIASLCLKSPGRFERI
LLRAANTHSSAKPCGQQSIHLGGVFELQPGASVFVNVTDPSQVSHGTGF
TSFGLLKL

In embodiments, a chimeric protein comprises a variant of a SIRPα(CD172a)-Fc-CD40L chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 60.

In embodiments, a heterologous chimeric protein comprises a first domain comprising a portion of SIRPα(CD172a), a second domain comprising a portion of OX40L, and a linker. In embodiments, the linker is a polypeptide selected from a flexible amino acid sequence, an IgG hinge region, and an antibody sequence. In embodiments, the linker comprises at least one cysteine residue capable of forming a disulfide bond and/or comprises a hinge-CH2-CH3 Fc domain. In embodiments, the linker comprises a hinge-CH2-CH3 Fc domain, e.g., from an IgG1 or from IgG4, including human IgG1 or IgG4. In embodiments, the linker comprises an amino acid sequence that is at least 95% identical to the amino acid sequence of SEQ ID NO: 1, SEQ ID NO: 2, or SEQ ID NO: 3. Thus, in embodiments, when a heterologous chimeric protein used in a method of the present disclosure comprises the extracellular domain of SIRPα(CD172a) (or a variant thereof), a linker comprising a hinge-CH2-CH3 Fc domain, and the extracellular domain of OX40L (or a variant thereof), it may be referred to herein as “SIRPα(CD172a)-Fc-OX40L”.

In embodiments, a SIRPα(CD172a)-Fc-OX40L chimeric protein of the present disclosure and/or a chimeric protein used in methods of the present disclosure has the following amino acid sequence:

(SEQ ID NO: 61)
EEELQVIQPDKSVLVAAGETATLRCTATSLIPVGPIQWFRGAGPGRELI
YNQKEGHFPRVTTVSDLTKRNNMDFSIRIGNITPADAGTYYCVKFRKGS
PDDVEFKSGAGTELSVRAKPSAPVVSGPAARATPQHTVSFTCESHGFSP
RDITLKWFKNGNELSDFQTNVDPVGESVSYSIHSTAKVVLTREDVHSQV
ICEVAHVTLQGDPLRGTANLSETIRVPPTLEVTQQPVRAENQVNVTCQV
RKFYPQRLQLTWLENGNVSRTETASTVTENKDGTYNWMSWLLVNVSAHR
DDVKLTCQVEHDGQPAVSKSHDLKVSAHPKEQGSNTAAENTGSNERNIY
SKYGPPCPPCPAPEFLGGPSVFLFPPKPKDQLMISRTPEVTCVVVDVSQ
EDPEVQFNWYVDGVEVHNAKTKPREEQFNSTYRVVSVLTVLHQDWLSGK
EYKCKVSSKGLPSSIEKTISNATGQPREPQVYTLPPSQEEMTKNQVSLT
CLVKGFYPSDIAVEWESNGQPENNYKTTPPVLDSDGSFFLYSRLTVDKS
RWQEGNVFSCSVLHEALHNHYTQKSLSLSLGKIEGRMDQVSHRYPRIQS
IKVQFTEYKKEKGFILTSQKEDEIMKVQNNSVIINCDGFYLISLKGYFS
QEVNISLHYQKDEEPLFQLKKVRSVNSLMVASLTYKDKVYLNVTTDNTS
LDDFHVNGGELILIHQNPGEFCVL

In embodiments, a chimeric protein comprises a variant of a SIRPα(CD172a)-Fc-OX40L chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 61.

In embodiments, a heterologous chimeric protein comprises a first domain comprising a portion of SIRPα(CD172a), a second domain comprising a portion of LIGHT, and a linker. In embodiments, the linker is a polypeptide selected from a flexible amino acid sequence, an IgG hinge region, and an antibody sequence. In embodiments, the linker comprises at least one cysteine residue capable of forming a disulfide bond and/or comprises a hinge-CH2-CH3 Fc domain. In embodiments, the linker comprises a hinge-CH2-CH3 Fc domain, e.g., from an IgG1 or from IgG4, including human IgG1 or IgG4. In embodiments, the linker comprises an amino acid sequence that is at least 95% identical to the amino acid sequence of SEQ ID NO: 1, SEQ ID NO: 2, or SEQ ID NO: 3. Thus, in embodiments, when a heterologous chimeric protein used in a method of the present disclosure comprises the extracellular domain of SIRPα(CD172a) (or a variant thereof), a linker comprising a hinge-CH2-CH3 Fc domain, and the extracellular domain of LIGHT (or a variant thereof), it may be referred to herein as “SIRPα(CD172a)-Fc-LIGHT”.

In embodiments, a SIRPα(CD172a)-Fc-LIGHT chimeric protein of the present disclosure and/or a chimeric protein used in methods of the present disclosure has the following amino acid sequence:

(SEQ ID NO: 63)
EEELQVIQPDKSVLVAAGETATLRCTATSLIPVGPIQWFRGAGPGRELI
YNQKEGHFPRVTTVSDLTKRNNMDFSIRIGNITPADAGTYYCVKFRKGS
PDDVEFKSGAGTELSVRAKPSAPVVSGPAARATPQHTVSFTCESHGFSP
RDITLKWFKNGNELSDFQTNVDPVGESVSYSIHSTAKVVLTREDVHSQV
ICEVAHVTLQGDPLRGTANLSETIRVPPTLEVTQQPVRAENQVNVTCQV
RKFYPQRLQLTWLENGNVSRTETASTVTENKDGTYNWMSWLLVNVSAHR
DDVKLTCQVEHDGQPAVSKSHDLKVSAHPKEQGSNTAAENTGSNERNIY
SKYGPPCPPCPAPEFLGGPSVFLFPPKPKDQLMISRTPEVTCVVVDVSQ
EDPEVQFNWYVDGVEVHNAKTKPREEQFNSTYRVVSVLTVLHQDWLSGK
EYKCKVSSKGLPSSIEKTISNATGQPREPQVYTLPPSQEEMTKNQVSLT
CLVKGFYPSDIAVEWESNGQPENNYKTTPPVLDSDGSFFLYSRLTVDKS
RWQEGNVFSCSVLHEALHNHYTQKSLSLSLGKIEGRMDLQLHWRLGEMV
TRLPDGPAGSWEQLIQERRSHEVNPAAHLTGANSSLTGSGGPLLWETQL
GLAFLRGLSYHDGALVVTKAGYYYIYSKVQLGGVGCPLGLASTITHGLY
KRTPRYPEELELLVSQQSPCGRATSSSRVWWDSSFLGGVVHLEAGEKVV
VRVLDERLVRLRDGTRSYFGAFMV

In embodiments, a chimeric protein comprises a variant of a SIRPα(CD172a)-Fc-LIGHT chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 63.

### The Second Pharmaceutical Composition

In one aspect, the present disclosure relates to a method for treating a cancer in a subject in need thereof comprising: (i) administering to the subject a first pharmaceutical composition of any of the embodiments disclosed herein; and (ii) administering to the subject a second pharmaceutical composition. In embodiments, the second pharmaceutical composition comprises an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof.

### The Hypomethylating Agents/Epigenetic Regulators Suitable in the Methods Disclosed Herein

In embodiments, the second pharmaceutical composition comprises a hypomethylating agent/epigenetic regulator. Epigenetic alternations concern the changes in histone or DNA modifications, such as DNA methylation, histone acetylation, and histone methylation, which regulate gene activity. Epigenetic dysregulation is associated with human disease, including cancer. Reviewed by Cheng et al., Targeting epigenetic regulators for cancer therapy: mechanisms and advances in clinical trials, Signal Transduction and Targeted Therapy 4: 62 (2019), the entire contents of which are hereby incorporated by reference. In embodiments, the hypomethylating agent/epigenetic regulator is a modulator of an enzyme selected from DNA methyltransferase (DNMT, e.g., DNMT1, DNMT2, DNMT3a, DNMT3b, and DNMT3L), histone methyltransferase, histone acetylase, histone deacetylase (HDAC) (e.g. one or more of HDAC1 to HDNAC11, and Sirt1-7), a DNA-demethylating enzyme, and a histone-demethylating enzyme. In embodiments the modulator is an inhibitor. In embodiments, the hypomethylating agent/epigenetic regulator is selected from azacitidine, 5-aza-2′-deoxycytidine, suberoylanilide hydroxamic acid (saha), romidepsin, belinostat, panobinostat, and chidamide. In embodiments, the hypomethylating agent/epigenetic regulator is azacitidine. Various suitable forms and formulations of azacitidine are disclosed in U.S. Pat. Nos. 4,684,630; 6,887,855; 6,943,249; 7,078,518; 7,772,199; 9,393,255; 9,765,108, the entire contents of each of which are hereby incorporated by reference.

### The Proteasomal Inhibitors Suitable in the Methods Disclosed Herein

The ubiquitin-mediated proteasome pathway is a central component of the cellular protein-degradation machinery with essential functions in homeostasis, which include preventing the accumulation deleterious proteins. Cancer cells produce proteins that promote both cell survival and proliferation, and/or inhibit mechanisms of cell death. Not surprisingly, studies have shown that proteasome inhibitors potently induce apoptosis in many types of cancer cells. Accordingly, in embodiments, the second pharmaceutical composition comprises a proteasomal inhibitor. In embodiments, the proteasomal inhibitors inhibit one or more of a chymotrypsin-like activity, a trypsin-like activity, and a peptidylglutamyl hydrolyzing activity present in the 20S core subunit of the proteasome. In embodiments, the proteasomal inhibitor is selected from bortezomib, carfilzomib and ixazomib. In embodiments, the proteasomal inhibitor is bortezomib. Bortezomib and formulations of bortezomib are disclosed in U.S. Pat. Nos. 5,780,454; 6,958,319; 6,713,446; 8,962,572, the entire contents of each of which are hereby incorporated by reference.

### The Anti-Metabolites Suitable in the Methods Disclosed Herein

Antimetabolites are commonly used in cancer treatment. Accordingly, in embodiments, the second pharmaceutical composition comprises an anti-metabolite. In embodiments, the antimetabolite interferes with the metabolism of a metabolite. In embodiments, the antimetabolite interferes with DNA replication and thereby inhibit cell division and tumor growth. In embodiments, the antimetabolite inhibits one or more enzymes selected from thymidylate synthase, DNA polymerase, RNA polymerase and nucleotide reductase. In embodiments, the antimetabolite is selected from 5-fluorouracil (5-FU), capecitabine, floxuridine, cytarabine (ARA-C), gemcitabine, decitabine, and vidaza. In embodiments, the antimetabolite is 5-fluorouracil (5-FU) or cytarabine (ARA-C). 5-fluorouracil (5-FU) and its formulations are disclosed in U.S. Pat. Nos. 2,802,005; 4,481,203; 4,622,325; 6,670,335, the entire contents of each of which are hereby incorporated by reference. Cytarabine and its formulations are disclosed in U.S. Pat. Nos. 3,116,282; and 8,431,806, the entire contents of each of which are hereby incorporated by reference.

### The DNA Synthesis Inhibitors Suitable in the Methods Disclosed Herein

In embodiments, the second pharmaceutical composition comprises a DNA synthesis inhibitor. In embodiments, the DNA Synthesis Inhibitor interferes with DNA replication and thereby inhibit cell division and tumor growth. In embodiments, the DNA synthesis inhibitor inhibits one or more enzymes selected from thymidylate synthase, DNA polymerase, and nucleotide reductase. In embodiments, the DNA synthesis inhibitor is selected from 5-fluorouracil (5-FU), capecitabine, floxuridine, cytarabine (ARA-C), gemcitabine, decitabine, and vidaza. In embodiments, the DNA synthesis inhibitor is 5-fluorouracil (5-FU) or cytarabine (ARA-C). 5-fluorouracil (5-FU) and its formulations are disclosed in U.S. Pat. Nos. 2,802,005; 4,481,203; 4,622,325; 6,670,335, the entire contents of each of which are hereby incorporated by reference. Cytarabine and its formulations are disclosed in U.S. Pat. Nos. 3,116,282; and 8,431,806, the entire contents of each of which are hereby incorporated by reference.

### The Immune Checkpoint Inhibitors Suitable in the Methods Disclosed Herein

In embodiments, the second pharmaceutical composition comprises an immune checkpoint inhibitor. In embodiments, the immune checkpoint inhibitor comprises an antibody capable of binding an immune checkpoint molecule. In embodiments, the antibody may be selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody. In embodiments, the antibody is a monoclonal antibody, e.g., a humanized monoclonal antibody. In embodiments, the immune checkpoint inhibitor comprises an agent that inhibits a pathway selected from CTLA-4, PD-1 and PD-L1. In embodiments, the immune checkpoint inhibitor comprises an anti-PD-L1 antibody. In embodiments, the anti-PD-L1 antibody is selected from atezolizumab, durvalumab, avelumab, envafolimab, BMS-936559, CK-301, CS-1001, SHR-1316 (HTI-1088), CBT-502 (TQB-2450) and BGB-A333. In embodiments, the immune checkpoint inhibitor comprises an anti-CTLA-4 antibody. In embodiments, the anti-CTLA-4 antibody is ipilimumab. In embodiments, the immune checkpoint inhibitor comprises an anti-PD-1 antibody selected from pembrolizumab, nivulomab and Cemiplimab.

### The Anthracyclines Suitable in the Methods Disclosed Herein

In embodiments, the second pharmaceutical composition comprises an anthracyline. In embodiments, the anthracycline interacts with DNA by intercalation and inhibits macromolecular biosynthesis. In embodiments, the anthracycline inhibits topoisomerase II. In embodiments, the anthracycline stabilizes the topoisomerase II complex after it has DNA chain cleavage. In embodiments, the anthracycline increases quinone type free radical production, contributing to its cytotoxicity. In embodiments, the anthracycline induces histone eviction from transcriptionally active chromatin. In embodiments, the anthracycline induces DNA damage response, and/or deregulation of epigenome and transcriptome. In embodiments, the anthracycline is selected from daunorubicin, doxorubicin, epirubicin, idarubicin, mitoxantrone, and valrubicin. In embodiments, the anthracycline is doxorubicin.

### The Topoisomerase II Inhibitors Suitable in the Methods Disclosed Herein

The nuclear enzyme DNA topoisomerase II is a major target for antineoplastic agents used in the treatment of a variety of cancers. Accordingly, in embodiments, the second pharmaceutical composition comprises a topoisomerase II inhibitor. In embodiments, the topoisomerase II inhibitor is selected from doxorubicin, epirubicin, valrubicin, daunorubicin, idarubicin, pitoxantrone, pixantrone, etoposide, teniposide, and amsacrine. In embodiments, the topoisomerase II inhibitor is doxorubicin. In embodiments, the doxorubicin interacts with DNA by intercalation and inhibits macromolecular biosynthesis. In embodiments, the doxorubicin stabilizes the topoisomerase II complex after it has DNA chain cleavage. In embodiments, the doxorubicin increases quinone type free radical production, contributing to its cytotoxicity. In embodiments, the doxorubicin induces histone eviction from transcriptionally active chromatin. In embodiments, the doxorubicin induces DNA damage response, and/or deregulation of epigenome and transcriptome.

### The Innate Immune Checkpoint Inhibitors Suitable in the Methods Disclosed Herein

In embodiments, the second pharmaceutical composition comprises an innate immune checkpoint inhibitor. In embodiments, the innate immune checkpoint inhibitor comprises agents that target CD47-SIRPα interaction. In embodiments, the innate immune checkpoint inhibitor is selected from magrolimab, CC-90002 (Celgene), CC-95251 (Celgene), TTI-621 (Trillium Therapeutics), TTI-622 (Trillium Therapeutics), ALX148 (ALX Oncology), SRF231 (Surface Oncology), IBI188 (Innovent), AO-176 (Arch Oncology), BI 765063/OSE-172 (Boehringer Ingelheim/OSE Immunotherapeutics), TG-1801/NI_1701 (TG Therapeutics/Novimmune), TJC4 (I-Mab) and the SIRPα-Fc-CD40L chimeric protein.

### The Bcl2 Inhibitors Suitable in the Methods Disclosed Herein

Bcl2 inhibitors have been shown to selectively induce apoptosis in malignant cells and have been extensively investigated as single agents and in combination with other drugs in several malignancies. Accordingly, in embodiments, the second pharmaceutical composition comprises a Bcl2 inhibitor. In embodiments, the Bcl2 inhibitor is selected from Oblimersen, Navitoclax (ABT-263), Venetoclax (ABT-199), Obatoclax mesylate (GX15-070), and AT-101. In embodiments, the Bcl2 inhibitor is venetoclax. Other suitable Bcl2 inhibitors are described in U.S. Pat. Nos. 8,546,399; 8,722,657; 9,174,982; 9,238,649; 9,539,251; 9,840,502; and 10,730,873, the entire contents of each of which are hereby incorporated by reference.

### The Protein Neddylation Inhibitors Suitable in the Methods Disclosed Herein

NEDD8 is a ubiquitin-like protein (ULP) that becomes covalently conjugated to a limited number of cellular proteins and alter their stability, subcellular localization and function. NEDD8-activating enzyme (NAE) plays an essential role in NEDD8 conjugation (“neddylation”). Neddylation drives tumor cells and also influences the functions of multiple important components of the tumor microenvironment (TME). In embodiments, the second pharmaceutical composition comprises a protein neddylation inhibitor. In embodiments, the protein neddylation inhibitor controls the activity of the cullin-RING subtype of ubiquitin ligases. In embodiments, the protein neddylation inhibitor regulates the turnover of a subset of proteins upstream of the proteasome. In embodiments, the protein neddylation inhibitor induces apoptosis, senescence and/or autophagy in cancer cells. Suitable protein neddylation inhibitors are disclosed in U.S. Pat. No. 8,207,177, the entire contents of which are hereby incorporated by reference. In embodiments, the protein neddylation inhibitor is pevonedistat.

### The Microtubule Targeting Agents Suitable in the Methods Disclosed Herein

The microtubule-targeting agents (MTAs) are a very successful class of cancer drugs with therapeutic benefits in both hematopoietic and solid tumors. In embodiments, the second pharmaceutical composition comprises a microtubule-targeting agent. In embodiments, the microtubule-targeting agent is a microtubule stabilizer. In embodiments, the microtubule-targeting agent is a microtubule destabilizer. In embodiments, the microtubule-targeting agent blocks the function of spindle. In embodiments, the microtubule-targeting agent exerts its inhibitory effects on cell proliferation primarily by blocking mitosis. In embodiments, the microtubule-targeting agent causes inhibition of the AKT/mTOR signaling pathway and thus inhibits cancer cell proliferation. In embodiments, the microtubule-targeting agent is selected from paclitaxel, epothilone, docetaxel, discodermolide, vinblastine, vincristine, vinorelbine, vinflunine, dolastatins, halichondrins, hemiasterlins, and cryptophysin 52. In embodiments, the microtubule-targeting agent is paclitaxel.

### The Thymidylate Synthase (TS) Inhibitor Suitable in the Methods Disclosed Herein

In embodiments, the second pharmaceutical composition comprises a thymidylate synthase (TS) inhibitor. In embodiments, the thymidylate synthase (TS) inhibitor interferes with DNA replication and thereby inhibit cell division and tumor growth. In embodiments, the thymidylate synthase (TS) inhibitor is selected from 5-fluorouracil (5-FU), 6-mercaptopurine (6-MP), capecitabine, cytarabine, floxuridine, fludarabine, gemcitabine, hydroxycarbamide, methotrexate, pemetrexed, phototrexate, raltitrexed, nolatrexed, ZD9331, and GS7904L. In embodiments, the DNA synthesis inhibitor is 5-fluorouracil (5-FU) or cytarabine (ARA-C). 5-fluorouracil (5-FU) and its formulations are disclosed in U.S. Pat. Nos. 2,802,005; 4,481,203; 4,622,325; 6,670,335, the entire contents of each of which are hereby incorporated by reference. Cytarabine and its formulations are disclosed in U.S. Pat. Nos. 3,116,282; and 8,431,806, the entire contents of each of which are hereby incorporated by reference.

### The Platinum Drugs Suitable in the Methods Disclosed Herein

Platinum drugs are widely used in the treatment of various cancers. In embodiments, the second pharmaceutical composition comprises a platinum drug. In embodiments, the platinum drug is selected from cisplatin, carboplatin, oxaliplatin, nedaplatin, heptaplatin and lobaplatin. In embodiments, the platinum drug is cisplatin. In embodiments, the platinum drug is oxaliplatin. Suitable platinum drugs and their formulations are described in U.S. Pat. Nos. 4,322,391; 4,915,956; 5,290,961; 5,338,874; 5,420,319; 5,716,988; 6,306,902; and 10,383,823, the entire contents of each of which are hereby incorporated by reference.

### The Topoisomerase I Inhibitors Suitable in the Methods Disclosed Herein

In embodiments, the second pharmaceutical composition comprises a topoisomerase I inhibitor. In embodiments, the topoisomerase I inhibitor is selected from camptothecin, belotecan topotecan, and irinotecan. In embodiments, the topoisomerase I inhibitor is irinotecan.

### The Anti-BCMA Antibody Suitable in the Methods Disclosed Herein

In embodiments, the second pharmaceutical composition comprises an anti-BCMA antibody. In embodiments, the anti-BCMA antibody is capable of antibody dependent cellular phagocytosis (ADCP). In embodiments, the anti-BCMA antibody may be selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody. In embodiments, the anti-BCMA antibody is a monoclonal antibody, e.g., a humanized monoclonal antibody. Suitable anti-BCMA antibodies are disclosed in WO 2010/104949, the entire contents of each of which are hereby incorporated by reference. In embodiments, the anti-BCMA antibody is C12A3.2, belantamab (including belantamab mafodotin). In embodiments, the anti-BCMA antibody is C12A3.2.

### The Anti-CD38 Antibody Suitable in the Methods Disclosed Herein

In embodiments, the second pharmaceutical composition comprises an anti-CD38 antibody. In embodiments, the anti-CD38 antibody is capable of antibody dependent cellular phagocytosis (ADCP). In embodiments, the anti-CD38 antibody may be selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody. In embodiments, the anti-CD38 antibody is a monoclonal antibody, e.g., a humanized monoclonal antibody. In embodiments, the anti-CD38 antibody is selected from daratumumab and isatuximab. In embodiments, the anti-CD38 antibody is daratumumab.

### The Immunomodulatory Imide Drugs (IMiDs) Suitable in the Methods Disclosed Herein

In embodiments, the second pharmaceutical composition comprises an immunomodulatory imide drug (IMiD). In embodiments, the immunomodulatory imide drug (IMiD) inhibits the production of tumor necrosis factor, interleukin 6 and immunoglobulin G and VEGF. In embodiments, the immunomodulatory imide drug (IMiD) co-stimulates T cells and NK cells. In embodiments, the immunomodulatory imide drug (IMiD) increases interferon gamma and interleukin 2 production. In embodiments, the immunomodulatory imide drug (IMiD) is selected from apremilast, thalidomide, lenalidomide, and pomalidomide. In embodiments, the immunomodulatory imide drug (IMiD) is lenalidomide or pomalidomide.

### The Anti-SLAMF7 Antibody Suitable in the Methods Disclosed Herein

In embodiments, the second pharmaceutical composition comprises an anti-SLAMF7 antibody. In embodiments, the anti-SLAMF7 antibody is capable of antibody dependent cellular phagocytosis (ADCP). In embodiments, the anti-SLAMF7 antibody may be selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody. In embodiments, the anti-SLAMF7 antibody is a monoclonal antibody, e.g., a humanized monoclonal antibody. In embodiments, the anti-SLAMF7 antibody is elotuzumab.

### The Reactivators of Mutated p53 Suitable in the Methods Disclosed Herein

Mutations of the tumor suppressor gene TP53 is very common in cancers. Many of the TP53 mutations cause the production of inactive p53 protein. In embodiments, the second pharmaceutical composition comprises a reactivator of mutated p53. In embodiments, the reactivator of mutated p53 is Prima-1 or APR-246. In embodiments, the APR-246 is spontaneously converted into the active species methylene quinuclidinone (MQ), which covalently binds to cysteine residues in mutant p53. In embodiments, the APR-246 produces thermo dynamic stabilization of mutant p53. In embodiments, the APR-246 shifts the equilibrium toward a functional conformation.

### The Anti-CD123 Antibody Suitable in the Methods Disclosed Herein

In embodiments, the second pharmaceutical composition comprises an anti-CD123 antibody. In embodiments, the anti-CD123 antibody is capable of antibody dependent cellular phagocytosis (ADCP). In embodiments, the anti-CD123 antibody may be selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody. In embodiments, the anti-CD123 antibody is a monoclonal antibody, e.g., a humanized monoclonal antibody. In embodiments, the anti-CD123 antibody is talacotuzumab.

### The Anti-FOLR1 Antibody Suitable in the Methods Disclosed Herein

In embodiments, the second pharmaceutical composition comprises an anti-FOLR1 antibody. In embodiments, the anti-FOLR1 antibody is capable of antibody dependent cellular phagocytosis (ADCP). In embodiments, the anti-FOLR1 antibody may be selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody. In embodiments, the anti-FOLR1 antibody is a monoclonal antibody, e.g., a humanized monoclonal antibody. In embodiments, the anti-FOLR1 antibody is farletuzumab or mirvetuximab soravtansine. In embodiments, the anti-FOLR1 antibody is farletuzumab.

### Azacitidine and Venetoclax

In embodiments, the second pharmaceutical composition comprises azacitidine and/or venetoclax, optionally wherein the azacitidine and venetoclax are contained in two separate dosage units, which are administered together or separately, optionally, sequentially.

### Diseases, Methods of Treatment, and Patient Selection

The methods comprise steps of administering to a subject in need thereof (either simultaneously or sequentially) an effective amount of an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or one or more chimeric proteins, in which each chimeric protein is capable of blocking immune inhibitory signals and/or stimulating immune activating signals.

It is often desirable to disrupt, block, reduce, inhibit, and/or sequester the transmission of immune inhibitory signals and, simultaneously or contemporaneously, enhance, increase, and/or stimulate the transmission of an immune stimulatory signal to an anti-cancer immune cell, to boost an immune response, for instance to enhance a patient's anti-tumor immune response.

In embodiments, the anticancer agent selected from a hypomethylating agent/epigenetic regulator, a proteasomal inhibitor, the anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure are capable of, or can be used in methods comprising, modulating the amplitude of an immune response, e.g., modulating the level of effector output.

In embodiments, e.g. when used for the treatment of cancer, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure alter the extent of immune stimulation as compared to immune inhibition to increase the amplitude of a T cell response, including, without limitation, stimulating increased levels of cytokine production, proliferation or target killing potential. In embodiments, the patient's T cells are activated and/or stimulated by the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure, with the activated T cells being capable of dividing and/or secreting cytokines.

Cancers or tumors refer to an uncontrolled growth of cells and/or abnormal increased cell survival and/or inhibition of apoptosis which interferes with the normal functioning of the bodily organs and systems. Included are benign and malignant cancers, polyps, hyperplasia, as well as dormant tumors or micrometastases. Also, included are cells having abnormal proliferation that is not impeded by the immune system (e.g., virus-infected cells). The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in a local area, forming a new tumor, which may be a local metastasis. The cancer may also be caused by a cancer cell that acquires the ability to penetrate the walls of lymphatic and/or blood vessels, after which the cancer cell is able to circulate through the bloodstream (thereby being a circulating tumor cell) to other sites and tissues in the body. The cancer may be due to a process such as lymphatic or hematogeneous spread. The cancer may also be caused by a tumor cell that comes to rest at another site, re-penetrates through the vessel or walls, continues to multiply, and eventually forms another clinically detectable tumor. The cancer may be this new tumor, which may be a metastatic (or secondary) tumor.

The cancer may be caused by tumor cells that have metastasized, which may be a secondary or metastatic tumor. The cells of the tumor may be like those in the original tumor. As an example, if a breast cancer or colon cancer metastasizes to the liver, the secondary tumor, while present in the liver, is made up of abnormal breast or colon cells, not of abnormal liver cells. The tumor in the liver may thus be a metastatic breast cancer or a metastatic colon cancer, not liver cancer.

The cancer may have an origin from any tissue. The cancer may originate from melanoma, colon, breast, or prostate; thus, the cancer may comprise cells that were originally skin, colon, breast, or prostate tissue, respectively. The cancer may also be a hematological malignancy, which may be leukemia or lymphoma. The cancer may invade a tissue such as liver, lung, bladder, or intestinal.

Representative cancers and/or tumors of the present disclosure include, but are not limited to, a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof used in methods of the present disclosure treat a subject that has a treatment-refractory cancer. In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure treat a subject that is refractory to one or more immune-modulating agents. For example, in embodiments, the an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure treat a subject that presents no response to treatment, or even progress, after 12 weeks or so of treatment. For instance, in embodiments, the subject is refractory to a PD-1 and/or PD-L1 and/or PD-L2 agent, including, for example, nivolumab (ONO-4538/BMS-936558, MDX1106, OPDIVO, BRISTOL MYERS SQUIBB), pembrolizumab (KEYTRUDA, MERCK), MK-3475 (MERCK), BMS 936559 (BRISTOL MYERS SQUIBB), Ibrutinib (PHARMACYCLICS/ABBVIE), atezolizumab (TECENTRIQ, GENENTECH), and/or MPDL3280A (ROCHE)-refractory patients. For instance, in embodiments, the subject is refractory to an anti-CTLA-4 agent, e.g., ipilimumab (YERVOY)-refractory patients (e.g., melanoma patients). Accordingly, in embodiments the present disclosure provides methods of cancer treatment that rescue patients that are non-responsive to various therapies, including monotherapy of one or more immune-modulating agents.

In embodiments, the present disclosure provides an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins which target a cell or tissue within the tumor microenvironment. In embodiments, the cell or tissue within the tumor microenvironment expresses one or more targets or binding partners of the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure. The tumor microenvironment refers to the cellular milieu, including cells, secreted proteins, physiological small molecules, and blood vessels in which the tumor exists. In embodiments, the cells or tissue within the tumor microenvironment are one or more of: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor. In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure targets a cancer cell. In embodiments, the cancer cell expresses one or more of targets or binding partners of the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure.

In embodiments, the present methods provide treatment with the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins in a patient who is refractory to an additional agent, such “additional agents” being disclosed elsewhere herein, inclusive, without limitation, of the various chemotherapeutic agents disclosed herein.

The activation of regulatory T cells is critically influenced by costimulatory and co-inhibitory signals. Two major families of costimulatory molecules include the B7 and the tumor necrosis factor (TNF) families. These molecules bind to receptors on T cells belonging to the CD28 or TNF receptor families, respectively. Many well-defined co-inhibitors and their receptors belong to the B7 and CD28 families.

In embodiments, an immune stimulatory signal refers to a signal that enhances an immune response. For example, in the context of oncology, such signals may enhance antitumor immunity. For instance, without limitation, immune stimulatory signal may be identified by directly stimulating proliferation, cytokine production, killing activity, or phagocytic activity of leukocytes. Specific examples include direct stimulation of TNF superfamily receptors such as OX40, CD40 and LIGHT using either receptor agonist antibodies or using a chimeric protein comprising the ligands for such receptors (OX40L, CD40L, and HVEM, respectively). Stimulation from any one of these receptors may directly stimulate the proliferation and cytokine production of individual T cell subsets. Another example includes direct stimulation of an immune inhibitory cell with through a receptor that inhibits the activity of such an immune suppressor cell. In another example, this would include stimulation of CD40 on the surface of an antigen-presenting cell using a CD40 agonist antibody or a chimeric protein comprising CD40L, causing activation of antigen presenting cells including enhanced ability of those cells to present antigen in the context of appropriate native costimulatory molecules, including those in the B7 or TNF superfamily. In another example, this would include stimulation of LTBR on the surface of a lymphoid or stromal cell using a LIGHT containing chimeric protein, causing activation of the lymphoid cell and/or production of pro-inflammatory cytokines or chemokines to further stimulate an immune response, optionally within a tumor.

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins are capable of, or find use in methods involving, enhancing, restoring, promoting and/or stimulating immune modulation. In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure described herein, restore, promote and/or stimulate the activity or activation of one or more immune cells against tumor cells including, but not limited to: T cells, cytotoxic T lymphocytes, T helper cells, natural killer (NK) cells, natural killer T (NKT) cells, anti-tumor macrophages (e.g. M1 macrophages), B cells, and dendritic cells. In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure enhance, restore, promote and/or stimulate the activity and/or activation of T cells, including, by way of a non-limiting example, activating and/or stimulating one or more T-cell intrinsic signals, including a pro-survival signal; an autocrine or paracrine growth signal; a p38 MAPK-, ERK-, STAT-, JAK-, AKT- or PI3K-mediated signal; an anti-apoptotic signal; and/or a signal promoting and/or necessary for one or more of: pro-inflammatory cytokine production or T cell migration or T cell tumor infiltration.

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure are capable of, or find use in methods involving, causing an increase of one or more of T cells (including without limitation cytotoxic T lymphocytes, T helper cells, natural killer T (NKT) cells), B cells, natural killer (NK) cells, natural killer T (NKT) cells, dendritic cells, monocytes, and macrophages (e.g., one or more of M1 and M2) into a tumor or the tumor microenvironment. In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure enhance recognition of tumor antigens by CD8+ T cells, particularly those T cells that have infiltrated into the tumor microenvironment. In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure induce CD19 expression and/or increases the number of CD19 positive cells (e.g., CD19 positive B cells). In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure induce IL-15Ra expression and/or increases the number of IL-15Ra positive cells (e.g., IL-15Ra positive dendritic cells).

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure are capable of, or find use in methods involving, inhibiting and/or causing a decrease in immunosuppressive cells (e.g., myeloid-derived suppressor cells (MDSCs), regulatory T cells (Tregs), tumor associated neutrophils (TANs), M2 macrophages, and tumor associated macrophages (TAMs), and particularly within the tumor and/or tumor microenvironment (TME). In embodiments, the present therapies may alter the ratio of M1 versus M2 macrophages in the tumor site and/or TME to favor M1 macrophages.

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure are able to increase the serum levels of various cytokines or chemokines including, but not limited to, one or more of IFNγ, TNFα, IL-2, IL-4, IL-5, IL-6, IL-7, IL-9, IL-10, IL-13, IL-15, IL-17A, IL-17F, IL-22, CCL2, CCL3, CCL4, CXCL8, CXCL9, CXCL10, CXCL11 and CXCL 12. In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure are capable of enhancing IL-2, IL-4, IL-5, IL-10, IL-13, IL-17A, IL-22, TNFα or IFNγ in the serum of a treated subject. In embodiments, administration of the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure is capable of enhancing TNFα secretion. In a specific embodiment, administration of the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure is capable of enhancing superantigen mediated TNFα secretion by leukocytes. Detection of such a cytokine response may provide a method to determine the optimal dosing regimen for the indicated anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure.

The antibodies directed to an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure are capable of increasing or preventing a decrease in a sub-population of CD4+ and/or CD8+ T cells.

The anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure are capable of enhancing tumor-killing activity by T cells.

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure inhibit, block and/or reduce cell death of an anti-tumor CD8+ and/or CD4+ T cell; or stimulate, induce, and/or increase cell death of a pro-tumor T cell. T cell exhaustion is a state of T cell dysfunction characterized by progressive loss of proliferative and effector functions, culminating in clonal deletion. Accordingly, a pro-tumor T cell refers to a state of T cell dysfunction that arises during many chronic infections, inflammatory diseases, and cancer. This dysfunction is defined by poor proliferative and/or effector functions, sustained expression of inhibitory receptors and a transcriptional state distinct from that of functional effector or memory T cells. Exhaustion prevents optimal control of infection and tumors. Illustrative pro-tumor T cells include, but are not limited to, Tregs, CD4+ and/or CD8+ T cells expressing one or more checkpoint inhibitory receptors, Th2 cells and Th17 cells. Checkpoint inhibitory receptors refer to receptors expressed on immune cells that prevent or inhibit uncontrolled immune responses. In contrast, an anti-tumor CD8+ and/or CD4+ T cell refers to T cells that can mount an immune response to a tumor.

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure are capable of, and can be used in methods comprising, increasing a ratio of effector T cells to regulatory T cells. Illustrative effector T cells include ICOS⁺ effector T cells; cytotoxic T cells (e.g., αβ TCR, CD3⁺, CD8⁺, CD45RO⁺); CD4⁺ effector T cells (e.g., αβ TCR, CD3⁺, CD4⁺, CCR7⁺, CD62Lhi, IL-7R/CD127⁺); CD8⁺ effector T cells (e.g., αβ TCR, CD3⁺, CD8⁺, CCR7⁺, CD62Lhi, IL-7R/CD127⁺); effector memory T cells (e.g., CD62Llow, CD44⁺, TCR, CD3⁺, IL-7R/CD127⁺, IL-15R⁺, CCR7low); central memory T cells (e.g., CCR7⁺, CD62L+, CD27⁺; or CCR7hi, CD44⁺, CD62Lhi, TCR, CD3⁺, IL-7R/CD127⁺, IL-15R⁺); CD62L⁺ effector T cells; CD8⁺ effector memory T cells (TEM) including early effector memory T cells (CD27⁺ CD62L⁻) and late effector memory T cells (CD27⁻ CD62L⁻) (TemE and TemL, respectively); CD127(⁺)CD25(low/−) effector T cells; CD127(⁻)CD25(⁻) effector T cells; CD8⁺ stem cell memory effector cells (TSCM) (e.g., CD44(low)CD62L(high)CD122(high)sca(⁺)); TH1 effector T-cells (e.g., CXCR3⁺, CXCR6⁺ and CCR5⁺; or αβ TCR, CD3⁺, CD4⁺, IL-12R⁺, IFNγR⁺, CXCR3⁺), TH2 effector T cells (e.g., CCR3⁺, CCR4⁺ and CCR8⁺; or αβ TCR, CD3⁺, CD4⁺, IL-4R⁺, IL-33R⁺, CCR4⁺, IL-17RB⁺, CRTH2⁺); TH9 effector T cells (e.g., αβ TCR, CD3⁺, CD4⁺); TH17 effector T cells (e.g., αβ TCR, CD3⁺, CD4⁺, IL-23R⁺, CCR6⁺, IL-1R⁺); CD4⁺CD45RO⁺CCR7⁺ effector T cells, CD4⁺CD45RO⁺CCR7(⁻) effector T cells; and effector T cells secreting IL-2, IL-4 and/or IFN-γ. Illustrative regulatory T cells include ICOS⁺ regulatory T cells, CD4⁺CD25+FOXP3⁺ regulatory T cells, CD4⁺CD25⁺ regulatory T cells, CD4⁺CD25-regulatory T cells, CD4⁺CD25high regulatory T cells, TIM-3+PD-1⁺ regulatory T cells, lymphocyte activation gene-3 (LAG-3)⁺ regulatory T cells, CTLA-4/CD152⁺ regulatory T cells, neuropilin-1 (Nrp-1)⁺ regulatory T cells, CCR4⁺CCR8⁺ regulatory T cells, CD62L (L-selectin)⁺ regulatory T cells, CD45RBlow regulatory T cells, CD127low regulatory T cells, LRRC32/GARP⁺ regulatory T cells, CD39⁺ regulatory T cells, GITR⁺ regulatory T cells, LAP⁺ regulatory T cells, 1B11⁺ regulatory T cells, BTLA⁺ regulatory T cells, type 1 regulatory T cells (Tr1 cells), T helper type 3 (Th3) cells, regulatory cell of natural killer T cell phenotype (NKTregs), CD8⁺ regulatory T cells, CD8⁺CD28⁻ regulatory T cells and/or regulatory T-cells secreting IL-10, IL-35, TGF-β, TNF-α, Galectin-1, IFN-γ and/or MCP1.

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure cause an increase in effector T cells (e.g., CD4⁺CD25-T cells).

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure cause a decrease in regulatory T cells (e.g., CD4⁺CD25⁺ T cells).

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure generate a memory response which may be capable of preventing relapse or protecting the animal from a recurrence and/or preventing, or reducing the likelihood of, metastasis. Thus, an animal treated with the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure is later able to attack tumor cells and/or prevent development of tumors when rechallenged after an initial treatment with the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure. Accordingly, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure stimulate both active tumor destruction and also immune recognition of tumor antigens, which are essential in programming a memory response capable of preventing relapse.

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure are capable of causing activation of antigen presenting cells. In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure are capable enhancing the ability of antigen presenting cells to present antigen.

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure are capable of, and can be used in methods comprising, transiently stimulating effector T cells for longer than about 12 hours, about 24 hours, about 48 hours, about 72 hours or about 96 hours or about 1 week or about 2 weeks. In embodiments, the transient stimulation of effector T cells occurs substantially in a patient's bloodstream or in a particular tissue/location including lymphoid tissues such as for example, the bone marrow, lymph-node, spleen, thymus, mucosa-associated lymphoid tissue (MALT), non-lymphoid tissues, or in the tumor microenvironment.

The chimeric proteins used in methods of the present disclosure unexpectedly provide binding of the extracellular domain components to their respective binding partners with slow off rates (Kd or K_{off}). In embodiments, this provides an unexpectedly long interaction of the receptor to ligand and vice versa. Such an effect allows for a longer positive signal effect, e.g., increase in or activation of immune stimulatory signals. For example, the chimeric proteins used in methods of the present disclosure, e.g., via the long off rate binding allows sufficient signal transmission to provide immune cell proliferation, allow for anti-tumor attack, allows sufficient signal transmission to provide release of stimulatory signals, e.g., cytokines.

The chimeric proteins used in methods of the present disclosure are capable of forming a stable synapse between cells. The stable synapse of cells promoted by the chimeric proteins (e.g., between cells bearing negative signals) provides spatial orientation to favor tumor reduction-such as positioning the T cells to attack tumor cells and/or sterically preventing the tumor cell from delivering negative signals, including negative signals beyond those masked by the chimeric proteins. In embodiments, this provides longer on-target (e.g., intra-tumoral) half-life (t_{1/2}) as compared to serum t_{1/2} of the chimeric proteins. Such properties could have the combined advantage of reducing off-target toxicities associated with systemic distribution of the chimeric proteins.

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure are capable of providing a sustained immunomodulatory effect.

The anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure provide synergistic therapeutic effects (e.g., anti-tumor effects) as it allows for improved site-specific interplay of two immunotherapy agents. In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof, and/or chimeric proteins used in methods of the present disclosure provide the potential for reducing off-site and/or systemic toxicity.

In embodiments, the chimeric proteins used in methods of the present disclosure exhibit enhanced safety profiles. In embodiment, the chimeric proteins used in methods of the present disclosure exhibit reduced toxicity profiles. For example, administration of the chimeric proteins used in methods of the present disclosure may result in reduced side effects such as one or more of diarrhea, inflammation (e.g., of the gut), or weight loss, which occur following administration of antibodies directed to the ligand(s)/receptor(s) targeted by the extracellular domains of the chimeric proteins used in methods of the present disclosure used in methods of the present disclosure. In embodiments, the chimeric proteins used in methods of the present disclosure provides improved safety, as compared to antibodies directed to the ligand(s)/receptor(s) targeted by the extracellular domains of the chimeric proteins used in methods of the present disclosure used in methods of the present disclosure, yet, without sacrificing efficacy.

In embodiments, the chimeric proteins used in methods of the present disclosure provide reduced side effects, e.g., GI complications, relative to current immunotherapies, e.g., antibodies directed to ligand(s)/receptor(s) targeted by the extracellular domains of the chimeric proteins used in methods of the present disclosure used in methods of the present disclosure. Illustrative GI complications include abdominal pain, appetite loss, autoimmune effects, constipation, cramping, dehydration, diarrhea, eating problems, fatigue, flatulence, fluid in the abdomen or ascites, gastrointestinal (GI) dysbiosis, GI mucositis, inflammatory bowel disease, irritable bowel syndrome (IBS-D and IBS-C), nausea, pain, stool or urine changes, ulcerative colitis, vomiting, weight gain from retaining fluid, and/or weakness.

In various aspects, the present disclosure provides compositions and methods that are useful for cancer immunotherapy. For instance, the present disclosure, in part, relates to methods for treating cancer comprising administering (either simultaneously or sequentially) a chimeric proteins disclosed herein and the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof.

In embodiments, the chimeric proteins of the present disclosure and/or chimeric proteins used in methods of the present disclosure eliminate or reduce side effects associated with disrupting the SIRP1a/CD47 signaling axis. In embodiments, the present chimeric proteins or methods utilizing the same eliminate or reduce hematological adverse effects. In embodiments, the present chimeric proteins or methods utilizing the same eliminate or reduce the extent of reductions in the number of circulating red blood cells and platelets, hemolysis, hemagglutination, thrombocytopenia, and/or anemia. In embodiments, the present chimeric proteins or methods utilizing the same demonstrate comparatively less hematological adverse effects than an anti-CD47 antibody.

An aspect of the present disclosure is a method for treating a cancer in a subject in need thereof. The method comprises steps of providing the subject a first pharmaceutical composition and providing the subject a second pharmaceutical composition. The first pharmaceutical composition comprises a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain. The second pharmaceutical composition comprises an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof.

In embodiments, the first pharmaceutical composition and the second pharmaceutical composition are administered simultaneously. In embodiments, the first pharmaceutical composition is administered after the second pharmaceutical composition is administered. In embodiments, the first pharmaceutical composition is administered before the second pharmaceutical composition is administered. In embodiments, the dose of the first pharmaceutical composition is less than the dose of the first pharmaceutical composition administered to a subject who has not undergone or is not undergoing treatment with the second pharmaceutical composition. In embodiments, the dose of the second pharmaceutical composition administered is less than the dose of the second pharmaceutical composition administered to a subject who has not undergone or is not undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition.

In embodiments, the first pharmaceutical composition and the second pharmaceutical composition are provided simultaneously, the first pharmaceutical composition is provided after the second pharmaceutical composition is provided, or the first pharmaceutical composition is provided before the second pharmaceutical composition is provided.

In embodiments, the dose of the first pharmaceutical composition is less than the dose of the first pharmaceutical composition provided to a subject who has not undergone or is not undergoing treatment with the second pharmaceutical composition.

In embodiments, the dose of the second pharmaceutical composition provided is less than the dose of the second pharmaceutical composition provided to a subject who has not undergone or is not undergoing treatment with the first pharmaceutical composition.

In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition.

In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition.

In embodiments, the heterologous chimeric protein comprises a first domain which comprises substantially the entire extracellular domain of SIRPα(CD172a) and/or a second domain which comprises substantially the entire extracellular domain of CD40L.

In embodiments, the heterologous chimeric protein comprises a first domain which comprises substantially the entire extracellular domain of SIRPα(CD172a) and/or a second domain which comprises substantially the entire extracellular domain of OX40L.

In embodiments, the heterologous chimeric protein comprises a first domain which comprises substantially the entire extracellular domain of SIRPα(CD172a) and/or a second domain which comprises substantially the entire extracellular domain of LIGHT.

In any of the embodiments disclosed herein, the heterologous chimeric protein comprises a first domain which comprises substantially the entire extracellular domain of SIRPα(CD172a) and/or a second domain which comprises substantially the entire extracellular domain of CD40L, OX40L, or LIGHT. In embodiments, the heterologous chimeric protein comprises: (a) a first domain comprising a portion of SIRPα(CD172a), (b) a second domain comprising a portion of CD40L, OX40L, or LIGHT, and (c) a linker comprising a hinge-CH2-CH3 Fc domain.

In any of the embodiments disclosed herein, the linker is a polypeptide selected from a flexible amino acid sequence, an IgG hinge region, and an antibody sequence. In embodiments, the linker comprises at least one cysteine residue capable of forming a disulfide bond and/or comprises a hinge-CH2-CH3 Fc domain. In embodiments, the linker comprises a hinge-CH2-CH3 Fc domain derived from IgG1 or IgG4, e.g., human IgG4 or human IgG4. In embodiments, the linker comprises an amino acid sequence that is at least 95% identical to the amino acid sequence of SEQ ID NO: 1, SEQ ID NO: 2, or SEQ ID NO: 3.

In embodiments, the first domain comprises an amino acid sequence that is at least 90%, or at least 93%, at least 95%, or at least 96%, or at least 98%, or at least 99% identical to the amino acid sequence of SEQ ID NO: 57.

In embodiments, the second domain comprises an amino acid sequence that is at least 90%, or at least 93%, at least 95%, or at least 96%, or at least 98%, or at least 99% identical to the amino acid sequence of SEQ ID NO: 58, SEQ ID NO: 59 or SEQ ID NO: 62. In embodiments, the second domain comprises an amino acid sequence that is at least 90%, or at least 93%, at least 95%, or at least 96%, or at least 98%, or at least 99% identical to the amino acid sequence of SEQ ID NO: 58.

In embodiments, the heterologous chimeric protein comprises an amino acid sequence that is at least 90%, or at least 93%, at least 95%, or at least 96%, or at least 98%, or at least 99% identical to the amino acid sequence of SEQ ID NO: 60, SEQ ID NO: 61, or SEQ ID NO: 63. In embodiments, the heterologous chimeric protein comprises an amino acid sequence that is at least 90%, or at least 93%, at least 95%, or at least 96%, or at least 98%, or at least 99% identical to the amino acid sequence of SEQ ID NO: 60.

In embodiments, the linker is a polypeptide selected from a flexible amino acid sequence, an IgG hinge region, and an antibody sequence.

In embodiments, the linker comprises at least one cysteine residue capable of forming a disulfide bond and/or comprises a hinge-CH2-CH3 Fc domain. In embodiments, the linker comprises a hinge-CH2-CH3 Fc domain derived from IgG4, e.g., human IgG4. In embodiments, the linker comprises an amino acid sequence that is at least 95% identical to the amino acid sequence of SEQ ID NO: 1, SEQ ID NO: 2, or SEQ ID NO: 3.

In embodiments, the heterologous chimeric protein comprises:
(a) a first domain comprising a portion of SIRPα(CD172a),
(b) a second domain comprising a portion of CD40L, and
(c) a linker comprising a hinge-CH2-CH3 Fc domain.

In embodiments, the heterologous chimeric protein comprises:
(a) a first domain comprising a portion of SIRPα(CD172a),
(b) a second domain comprising a portion of OX40L, and
(c) a linker comprising a hinge-CH2-CH3 Fc domain.

In embodiments, the heterologous chimeric protein comprises:
(a) a first domain comprising a portion of SIRPα(CD172a),
(b) a second domain comprising a portion of LIGHT, and
(c) a linker comprising a hinge-CH2-CH3 Fc domain.

In embodiments, the hypomethylating agent/epigenetic regulator is selected from azacitidine, 5-aza-2′-deoxycytidine, suberoylanilide hydroxamic acid (saha), romidepsin, belinostat, panobinostat, and chidamide. In embodiments, the hypomethylating agent/epigenetic regulator is azacitidine.

In embodiments, the proteasomal inhibitor is selected from bortezomib, carfilzomib and ixazomib. In embodiments, the proteasomal inhibitor is bortezomib.

In embodiments, the antimetabolite inhibits one or more enzymes selected from thymidylate synthase, DNA polymerase, RNA polymerase and nucleotide reductase. In embodiments, the antimetabolite is selected from 5-fluorouracil (5-FU), capecitabine, floxuridine, cytarabine (ARA-C), gemcitabine, decitabine, and vidaza. In embodiments, the antimetabolite is 5-fluorouracil (5-FU) or cytarabine (ARA-C).

In embodiments, the DNA synthesis inhibitor is selected from 5-fluorouracil (5-FU), capecitabine, floxuridine, cytarabine (ARA-C), gemcitabine, decitabine, and vidaza. In embodiments, the DNA synthesis inhibitor is 5-fluorouracil (5-FU) or cytarabine (ARA-C).

In embodiments, the immune checkpoint inhibitor comprises an agent that inhibits a pathway selected from CTLA-4, PD-1 and PD-L1. In embodiments, the immune checkpoint inhibitor comprises an anti-PD-L1 antibody. In embodiments, the anti-PD-L1 antibody is selected from atezolizumab, durvalumab, avelumab, envafolimab, BMS-936559, CK-301, CS-1001, SHR-1316 (HTI-1088), CBT-502 (TQB-2450) and BGB-A333. In embodiments, the immune checkpoint inhibitor comprises an anti-CTLA-4 antibody. In embodiments, the anti-CTLA-4 antibody is ipilimumab. In embodiments, the immune checkpoint inhibitor comprises an anti-PD-1 antibody selected from pembrolizumab, nivulomab and cemiplimab.

In embodiments, the anthracycline is selected from daunorubicin, doxorubicin, epirubicin, idarubicin, mitoxantrone, and valrubicin. In embodiments, the anthracycline is doxorubicin.

In embodiments, the topoisomerase II inhibitor is selected from doxorubicin, epirubicin, valrubicin, daunorubicin, idarubicin, pitoxantrone, pixantrone, etoposide, teniposide, and amsacrine. In embodiments, the topoisomerase II inhibitor is doxorubicin.

In embodiments, the innate immune checkpoint inhibitor comprises agents that target CD47-SIRPα interaction. In embodiments, the innate immune checkpoint inhibitor is selected from magrolimab, CC-90002 (Celgene), CC-95251 (Celgene), TTI-621 (Trillium Therapeutics), TTI-622 (Trillium Therapeutics), ALX148 (ALX Oncology), SRF231 (Surface Oncology), IBI188 (Innovent), AO-176 (Arch Oncology), BI 765063/OSE-172 (Boehringer Ingelheim/OSE Immunotherapeutics), and TG-1801/NI_1701 (TG Therapeutics/Novimmune), TJC4 (I-Mab).

In embodiments, the Bcl2 inhibitor is selected from Oblimersen, Navitoclax (ABT-263), Venetoclax (ABT-199), Obatoclax mesylate (GX15-070), and AT-101. In embodiments, the Bcl2 inhibitor is venetoclax.

In embodiments, the protein neddylation inhibitor is pevonedistat.

In embodiments, the microtubule-targeting agent is selected from paclitaxel, epothilone, docetaxel, discodermolide, vinblastine, vincristine, vinorelbine, vinflunine, dolastatins, halichondrins, hemiasterlins, and cryptophysin 52. In embodiments, the microtubule-targeting agent is paclitaxel.

In embodiments, the thymidylate synthase (TS) inhibitor is selected from 5-fluorouracil (5-FU), 6-mercaptopurine (6-MP), capecitabine, cytarabine, floxuridine, fludarabine, gemcitabine, hydroxycarbamide, methotrexate, pemetrexed, phototrexate, raltitrexed, nolatrexed, ZD9331, and GS7904L. In embodiments, the DNA synthesis inhibitor is 5-fluorouracil (5-FU) or cytarabine (ARA-C).

In embodiments, the platinum drug is selected from cisplatin, carboplatin, oxaliplatin, nedaplatin, heptaplatin and lobaplatin. In embodiments, the platinum drug is cisplatin. In embodiments, the platinum drug is oxaliplatin.

In embodiments, the topoisomerase I inhibitor is selected from camptothecin, belotecan topotecan, and irinotecan. In embodiments, the topoisomerase I inhibitor is irinotecan.

In embodiments, the anti-BCMA antibody is C12A3.2.

In embodiments, the anti-CD38 antibody is selected from daratumumab and isatuximab. In embodiments, the anti-CD38 antibody is daratumumab.

In embodiments, the immunomodulatory imide drug (IMiD) is selected from apremilast, thalidomide, lenalidomide, and pomalidomide. In embodiments, the immunomodulatory imide drug (IMiD) is lenalidomide or pomalidomide.

In embodiments, the anti-SLAMF7 antibody is elotuzumab.

In embodiments, the reactivator of mutated p53 is Prima-1 or APR-246.

In embodiments, the anti-CD123 antibody is talacotuzumab.

In embodiments, the anti-FOLR1 antibody is farletuzumab or mirvetuximab.

In embodiments, the cancer is or is related to a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In embodiments, the subject has a cancer that is poorly responsive or is refractory to treatment comprising an antibody that is capable of binding PD-1 or binding a PD-1 ligand. In embodiments, the cancer is poorly responsive or is non-responsive to treatment with an antibody that is capable of binding PD-1 or binding a PD-1 ligand after 12 weeks or so of such treatment. In embodiments, the antibody that is capable of binding PD-1 or binding a PD-1 ligand is selected from the group consisting of nivolumab (ONO-4538/BMS-936558, MDX1106, OPDIVO, BRISTOL MYERS SQUIBB), pembrolizumab (KEYTRUDA, MERCK), RMP1-14, AGEN2034 (AGENUS), cemiplimab (REGN-2810), MK-3475 (MERCK), BMS 936559 (BRISTOL MYERS SQUIBB), Ibrutinib (PHARMACYCLICS/ABBVIE), atezolizumab (TECENTRIQ, GENENTECH), and MPDL3280A (ROCHE).

Another aspect of the present disclosure is method for treating a cancer in a subject comprising providing the subject a pharmaceutical composition comprising a heterologous chimeric protein. The heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain. In this aspect, the subject has undergone or is undergoing treatment with anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof.

In embodiments, the dose of the pharmaceutical composition provided to the subject is less than the dose of the pharmaceutical composition that is provided to a subject who has not undergone or is not undergoing treatment with the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof.

Yet another aspect of the present disclosure is a method for treating a cancer in a subject comprising providing the subject a pharmaceutical composition comprising anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof. In this aspect, the subject has undergone or is undergoing treatment with: a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain.

In embodiments, the dose of the pharmaceutical composition provided to the subject is less than the dose of the pharmaceutical composition that is provided to a subject who has not undergone or is not undergoing treatment with the heterologous chimeric protein.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising a hypomethylating agent/epigenetic regulator. In embodiments, the hypomethylating agent/epigenetic regulator is selected from azacitidine, 5-aza-2′-deoxycytidine, suberoylanilide hydroxamic acid (saha), romidepsin, belinostat, panobinostat, and chidamide. In embodiments, the hypomethylating agent/epigenetic regulator is azacitidine.

In one aspect, the present disclosure provides a method for treating a cancer in a subject in need thereof comprising: administering to the subject a second pharmaceutical composition comprising a hypomethylating agent/epigenetic regulator, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the hypomethylating agent/epigenetic regulator is selected from azacitidine, 5-aza-2′-deoxycytidine, suberoylanilide hydroxamic acid (saha), romidepsin, belinostat, panobinostat, and chidamide. In embodiments, the hypomethylating agent/epigenetic regulator is azacitidine.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising a proteasomal inhibitor. In embodiments, the proteasomal inhibitor is selected from bortezomib, carfilzomib and ixazomib. In embodiments, the proteasomal inhibitor is bortezomib.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising a proteasomal inhibitor. In embodiments, the proteasomal inhibitor is selected from bortezomib, carfilzomib and ixazomib. In embodiments, the proteasomal inhibitor is bortezomib.

In one aspect, the present disclosure provides a method for treating a cancer in a subject in need thereof comprising: administering to the subject a second pharmaceutical composition comprising a proteasomal inhibitor, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the proteasomal inhibitor is selected from bortezomib, carfilzomib and ixazomib. In embodiments, the proteasomal inhibitor is bortezomib.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising an anti-metabolite. In embodiments, the antimetabolite is selected from 5-fluorouracil (5-FU), capecitabine, floxuridine, cytarabine (ARA-C), gemcitabine, decitabine, and vidaza. In embodiments, the antimetabolite is cytarabine (ARA-C).

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising an anti-metabolite. In embodiments, the antimetabolite is selected from 5-fluorouracil (5-FU), capecitabine, floxuridine, cytarabine (ARA-C), gemcitabine, decitabine, and vidaza. In embodiments, the antimetabolite is cytarabine (ARA-C).

In one aspect, the present disclosure provides a method for treating a cancer in a subject in need thereof comprising: administering to the subject a second pharmaceutical composition comprising an anti-metabolite, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the antimetabolite is selected from 5-fluorouracil (5-FU), capecitabine, floxuridine, cytarabine (ARA-C), gemcitabine, decitabine, and vidaza. In embodiments, the antimetabolite is cytarabine (ARA-C).

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising a DNA synthesis inhibitor. In embodiments, the DNA synthesis inhibitor is selected from 5-fluorouracil (5-FU), capecitabine, floxuridine, cytarabine (ARA-C), gemcitabine, decitabine, and vidaza. In embodiments, the DNA synthesis inhibitor is cytarabine (ARA-C) or 5-fluorouracil (5-FU).

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising a DNA synthesis inhibitor. In embodiments, the DNA synthesis inhibitor is selected from 5-fluorouracil (5-FU), capecitabine, floxuridine, cytarabine (ARA-C), gemcitabine, decitabine, and vidaza. In embodiments, the DNA synthesis inhibitor is cytarabine (ARA-C) or 5-fluorouracil (5-FU).

In one aspect, the present disclosure provides a method for treating a cancer in a subject in need thereof comprising: administering the subject a second pharmaceutical composition comprising a DNA synthesis inhibitor, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the DNA synthesis inhibitor is selected from 5-fluorouracil (5-FU), capecitabine, floxuridine, cytarabine (ARA-C), gemcitabine, decitabine, and vidaza. In embodiments, the DNA synthesis inhibitor is cytarabine (ARA-C) or 5-fluorouracil (5-FU).

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising an immune checkpoint inhibitor. In embodiments, the immune checkpoint inhibitor comprises an agent that inhibits a pathway selected from CTLA-4, PD-1 and PD-L1. In embodiments, the immune checkpoint inhibitor comprises an anti-PD-L1 antibody. In embodiments, the anti-PD-L1 antibody is selected from atezolizumab, durvalumab, avelumab, envafolimab, BMS-936559, CK-301, CS-1001, SHR-1316 (HTI-1088), CBT-502 (TQB-2450) and BGB-A333.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising an immune checkpoint inhibitor. In embodiments, the immune checkpoint inhibitor comprises an agent that inhibits a pathway selected from CTLA-4, PD-1 and PD-L1. In embodiments, the immune checkpoint inhibitor comprises an anti-PD-L1 antibody. In embodiments, the anti-PD-L1 antibody is selected from atezolizumab, durvalumab, avelumab, envafolimab, BMS-936559, CK-301, CS-1001, SHR-1316 (HTI-1088), CBT-502 (TQB-2450) and BGB-A333.

In one aspect, the present disclosure provides a method for treating a cancer in a subject in need thereof comprising: administering to the subject a second pharmaceutical composition comprising an immune checkpoint inhibitor, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the immune checkpoint inhibitor comprises an agent that inhibits a pathway selected from CTLA-4, PD-1 and PD-L1. In embodiments, the immune checkpoint inhibitor comprises an anti-PD-L1 antibody. In embodiments, the anti-PD-L1 antibody is selected from atezolizumab, durvalumab, avelumab, envafolimab, BMS-936559, CK-301, CS-1001, SHR-1316 (HTI-1088), CBT-502 (TQB-2450) and BGB-A333.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising an anthracycline. In embodiments, the anthracycline is selected from daunorubicin, doxorubicin, epirubicin, idarubicin, mitoxantrone, and valrubicin In embodiments, the anthracycline is doxorubicin.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising an anthracycline. In embodiments, the anthracycline is selected from daunorubicin, doxorubicin, epirubicin, idarubicin, mitoxantrone, and valrubicin In embodiments, the anthracycline is doxorubicin.

In one aspect, the present disclosure provides a method for treating a cancer in a subject in need thereof comprising: administering to the subject a second pharmaceutical composition comprising an anthracycline, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the anthracycline is selected from daunorubicin, doxorubicin, epirubicin, idarubicin, mitoxantrone, and valrubicin In embodiments, the anthracycline is doxorubicin.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising a topoisomerase II inhibitor. In embodiments, the topoisomerase II inhibitor is selected from doxorubicin, epirubicin, valrubicin, daunorubicin, idarubicin, pitoxantrone, pixantrone, etoposide, teniposide, and amsacrine. In embodiments, the topoisomerase II inhibitor is doxorubicin.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising a topoisomerase II inhibitor. In embodiments, the topoisomerase II inhibitor is selected from doxorubicin, epirubicin, valrubicin, daunorubicin, idarubicin, pitoxantrone, pixantrone, etoposide, teniposide, and amsacrine. In embodiments, the topoisomerase II inhibitor is doxorubicin.

In one aspect, the present disclosure provides a method for treating a cancer in a subject in need thereof comprising: administering to the subject a second pharmaceutical composition comprising a topoisomerase II inhibitor, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the topoisomerase II inhibitor is selected from doxorubicin, epirubicin, valrubicin, daunorubicin, idarubicin, pitoxantrone, pixantrone, etoposide, teniposide, and amsacrine. In embodiments, the topoisomerase II inhibitor is doxorubicin.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising an innate immune checkpoint inhibitor. In embodiments, the innate immune checkpoint inhibitor comprises an agent that target CD47-SIRPα interaction. In embodiments, the innate immune checkpoint inhibitor is selected from magrolimab, CC-90002 (Celgene), CC-95251 (Celgene), TTI-621 (Trillium Therapeutics), TTI-622 (Trillium Therapeutics), ALX148 (ALX Oncology), SRF231 (Surface Oncology), IBI188 (Innovent), AO-176 (Arch Oncology), BI 765063/OSE-172 (Boehringer Ingelheim/OSE Immunotherapeutics), TG-1801/NI_1701 (TG Therapeutics/Novimmune), TJC4 (I-Mab) and the SIRPα-Fc-CD40L chimeric protein.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising an innate immune checkpoint inhibitor. In embodiments, the innate immune checkpoint inhibitor comprises an agent that target CD47-SIRPα interaction. In embodiments, the innate immune checkpoint inhibitor is selected from magrolimab, CC-90002 (Celgene), CC-95251 (Celgene), TTI-621 (Trillium Therapeutics), TTI-622 (Trillium Therapeutics), ALX148 (ALX Oncology), SRF231 (Surface Oncology), IBI188 (Innovent), AO-176 (Arch Oncology), BI 765063/OSE-172 (Boehringer Ingelheim/OSE Immunotherapeutics), TG-1801/NI_1701 (TG Therapeutics/Novimmune), TJC4 (I-Mab) and the SIRPα-Fc-CD40L chimeric protein.

In one aspect, the present disclosure provides a method for treating a cancer in a subject in need thereof comprising: administering to the subject a second pharmaceutical composition comprising an innate immune checkpoint inhibitor, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the innate immune checkpoint inhibitor comprises an agent that target CD47-SIRPα interaction. In embodiments, the innate immune checkpoint inhibitor is selected from magrolimab, CC-90002 (Celgene), CC-95251 (Celgene), TTI-621 (Trillium Therapeutics), TTI-622 (Trillium Therapeutics), ALX148 (ALX Oncology), SRF231 (Surface Oncology), IBI188 (Innovent), AO-176 (Arch Oncology), BI 765063/OSE-172 (Boehringer Ingelheim/OSE Immunotherapeutics), TG-1801/NI_1701 (TG Therapeutics/Novimmune), TJC4 (I-Mab) and the SIRPα-Fc-CD40L chimeric protein.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising a Bcl2 inhibitor. In embodiments, the Bcl2 inhibitor is selected from oblimersen, navitoclax (ABT-263), venetoclax (ABT-199), obatoclax mesylate (GX15-070), and AT-101. In embodiments, the Bcl2 inhibitor is venetoclax.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising a Bcl2 inhibitor. In embodiments, the Bcl2 inhibitor is selected from oblimersen, navitoclax (ABT-263), venetoclax (ABT-199), obatoclax mesylate (GX15-070), and AT-101. In embodiments, the Bcl2 inhibitor is venetoclax.

In one aspect, the present disclosure provides a method for treating a cancer in a subject in need thereof comprising: administering to the subject a second pharmaceutical composition comprising a Bcl2 inhibitor, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the Bcl2 inhibitor is selected from oblimersen, navitoclax (ABT-263), venetoclax (ABT-199), obatoclax mesylate (GX15-070), and AT-101. In embodiments, the Bcl2 inhibitor is venetoclax.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising a protein neddylation inhibitor. In embodiments, the protein neddylation inhibitor is pevonedistat (MLN4924).

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising a protein neddylation inhibitor. In embodiments, the protein neddylation inhibitor is pevonedistat (MLN4924).

In one aspect, the present disclosure provides a method for treating a cancer in a subject in need thereof comprising: administering to the subject a second pharmaceutical composition comprising a protein neddylation inhibitor, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the protein neddylation inhibitor is pevonedistat (MLN4924).

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising a microtubule-targeting agent. In embodiments, the microtubule-targeting agent is selected from paclitaxel, epothilone, docetaxel, discodermolide, vinblastine, vincristine, vinorelbine, vinflunine, dolastatins, halichondrins, hemiasterlins, and cryptophysin 52. In embodiments, the microtubule-targeting agent is paclitaxel.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising a microtubule-targeting agent. In embodiments, the microtubule-targeting agent is selected from paclitaxel, epothilone, docetaxel, discodermolide, vinblastine, vincristine, vinorelbine, vinflunine, dolastatins, halichondrins, hemiasterlins, and cryptophysin 52. In embodiments, the microtubule-targeting agent is paclitaxel.

In one aspect, the present disclosure provides a method for treating a cancer in a subject in need thereof comprising: administering to the subject a second pharmaceutical composition comprising a microtubule-targeting agent, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the microtubule-targeting agent is selected from paclitaxel, epothilone, docetaxel, discodermolide, vinblastine, vincristine, vinorelbine, vinflunine, dolastatins, halichondrins, hemiasterlins, and cryptophysin 52. In embodiments, the microtubule-targeting agent is paclitaxel.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising a thymidylate synthase (TS) inhibitor. In embodiments, the thymidylate synthase (TS) inhibitor is selected from 5-fluorouracil (5-FU), 6-mercaptopurine (6-MP), capecitabine, cytarabine, floxuridine, fludarabine, gemcitabine, hydroxycarbamide, methotrexate, pemetrexed, phototrexate, raltitrexed, nolatrexed, ZD9331, and GS7904L. In embodiments, the thymidylate synthase (TS) inhibitor is 5-fluorouracil (5-FU).

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising a thymidylate synthase (TS) inhibitor. In embodiments, the thymidylate synthase (TS) inhibitor is selected from 5-fluorouracil (5-FU), 6-mercaptopurine (6-MP), capecitabine, cytarabine, floxuridine, fludarabine, gemcitabine, hydroxycarbamide, methotrexate, pemetrexed, phototrexate, raltitrexed, nolatrexed, ZD9331, and GS7904L. In embodiments, the thymidylate synthase (TS) inhibitor is 5-fluorouracil (5-FU).

In one aspect, the present disclosure provides a method for treating a cancer in a subject in need thereof comprising: administering to the subject a second pharmaceutical composition comprising a thymidylate synthase (TS) inhibitor, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the thymidylate synthase (TS) inhibitor is selected from 5-fluorouracil (5-FU), 6-mercaptopurine (6-MP), capecitabine, cytarabine, floxuridine, fludarabine, gemcitabine, hydroxycarbamide, methotrexate, pemetrexed, phototrexate, raltitrexed, nolatrexed, ZD9331, and GS7904L. In embodiments, the thymidylate synthase (TS) inhibitor is 5-fluorouracil (5-FU).

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising a platinum drug. In embodiments, the platinum drug is selected from cisplatin, carboplatin, oxaliplatin, nedaplatin, heptaplatin and lobaplatin. In embodiments, the platinum drug is cisplatin. In embodiments, the platinum drug is oxaliplatin.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising a platinum drug. In embodiments, the platinum drug is selected from cisplatin, carboplatin, oxaliplatin, nedaplatin, heptaplatin and lobaplatin. In embodiments, the platinum drug is cisplatin. In embodiments, the platinum drug is oxaliplatin.

In one aspect, the present disclosure provides a method for treating a cancer in a subject in need thereof comprising: administering to the subject a second pharmaceutical composition comprising a platinum drug, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the platinum drug is selected from cisplatin, carboplatin, oxaliplatin, nedaplatin, heptaplatin and lobaplatin. In embodiments, the platinum drug is cisplatin. In embodiments, the platinum drug is oxaliplatin.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising a topoisomerase I inhibitor. In embodiments, the topoisomerase I inhibitor is selected from camptothecin, belotecan topotecan, and irinotecan. In embodiments, the topoisomerase I inhibitor is irinotecan.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising a topoisomerase I inhibitor. In embodiments, the topoisomerase I inhibitor is selected from camptothecin, belotecan topotecan, and irinotecan. In embodiments, the topoisomerase I inhibitor is irinotecan.

In one aspect, the present disclosure provides a method for treating a cancer in a subject in need thereof comprising: administering to the subject a second pharmaceutical composition comprising a topoisomerase I inhibitor, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the topoisomerase I inhibitor is selected from camptothecin, belotecan topotecan, and irinotecan. In embodiments, the topoisomerase I inhibitor is irinotecan.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising an anti-BCMA antibody. In embodiments, the anti-BCMA antibody is C12A3.2.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising an anti-BCMA antibody. In embodiments, the anti-BCMA antibody is belantamab or C12A3.2. In embodiments, the anti-BCMA antibody is C12A3.2.

In one aspect, the present disclosure provides a method for treating a cancer in a subject in need thereof comprising: administering to the subject a second pharmaceutical composition comprising an anti-BCMA antibody, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the anti-BCMA antibody is belantamab. In embodiments, the anti-BCMA antibody is belantamab or C12A3.2.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising an anti-CD38 antibody. In embodiments, the anti-CD38 antibody is selected from daratumumab and isatuximab. In embodiments, the anti-CD38 antibody is daratumumab.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising an anti-CD38 antibody. In embodiments, the anti-CD38 antibody is selected from daratumumab and isatuximab. In embodiments, the anti-CD38 antibody is daratumumab.

In one aspect, the present disclosure provides a method for treating a cancer in a subject in need thereof comprising: administering to the subject a second pharmaceutical composition comprising an anti-CD38 antibody, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the anti-CD38 antibody is selected from daratumumab and isatuximab. In embodiments, the anti-CD38 antibody is daratumumab.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising an immunomodulatory imide drug (IMiD). In embodiments, the immunomodulatory imide drug (IMiD) is selected from apremilast, thalidomide, lenalidomide, and pomalidomide. In embodiments, the immunomodulatory imide drug (IMiD) is lenalidomide or pomalidomide.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising an immunomodulatory imide drug (IMiD). In embodiments, the immunomodulatory imide drug (IMiD) is selected from apremilast, thalidomide, lenalidomide, and pomalidomide. In embodiments, the immunomodulatory imide drug (IMiD) is lenalidomide or pomalidomide.

In one aspect, the present disclosure provides a method for treating a cancer in a subject in need thereof comprising: administering to the subject a second pharmaceutical composition comprising an immunomodulatory imide drug (IMiD), wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the immunomodulatory imide drug (IMiD) is selected from apremilast, thalidomide, lenalidomide, and pomalidomide. In embodiments, the immunomodulatory imide drug (IMiD) is lenalidomide or pomalidomide.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising an anti-SLAMF7 antibody.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising an anti-SLAMF7 antibody.

In one aspect, the present disclosure provides a method for treating a cancer in a subject in need thereof comprising: administering to the subject a second pharmaceutical composition comprising an anti-SLAMF7 antibody, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the anti-SLAMF7 antibody is elotuzumab.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising an anti-CD123 antibody. In embodiments, the anti-CD123 antibody is talacotuzumab.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising an anti-CD123 antibody. In embodiments, the anti-CD123 antibody is talacotuzumab.

In one aspect, the present disclosure provides a method for treating a cancer in a subject in need thereof comprising: administering to the subject a second pharmaceutical composition comprising an anti-CD123 antibody, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the anti-CD123 antibody is talacotuzumab.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising a reactivator of mutated p53. In embodiments, the reactivator of mutated p53 is Prima-1 or APR-246. In embodiments, the reactivator of mutated p53 is APR-246.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising a reactivator of mutated p53. In embodiments, the reactivator of mutated p53 is Prima-1 or APR-246. In embodiments, the reactivator of mutated p53 is APR-246.

In one aspect, the present disclosure provides a method for treating a cancer in a subject in need thereof comprising: administering to the subject a second pharmaceutical composition comprising a reactivator of mutated p53, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the reactivator of mutated p53 is Prima-1 or APR-246. In embodiments, the reactivator of mutated p53 is APR-246.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain; and administering to the subject a second pharmaceutical composition comprising an anti-FOLR1 antibody. In embodiments, the anti-FOLR1 antibody is farletuzumab or mirvetuximab. In embodiments, the anti-FOLR1 antibody is farletuzumab.

In one aspect, the present disclosure provides a method for treating a cancer in a subject in need thereof comprising: administering to the subject a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain, wherein the subject has undergone or is undergoing treatment with a second pharmaceutical composition comprising an anti-FOLR1 antibody. In embodiments, the anti-FOLR1 antibody is farletuzumab or mirvetuximab. In embodiments, the anti-FOLR1 antibody is farletuzumab.

In one aspect, the present disclosure provides a method for treating a cancer in a subject in need thereof comprising: administering to the subject a second pharmaceutical composition comprising an anti-FOLR1 antibody, wherein the subject has undergone or is undergoing treatment with a first pharmaceutical composition comprising a heterologous chimeric protein comprising: a first domain comprising a portion of the extracellular domain of SIRPα(CD172a), wherein the portion is capable of binding a SIRPα(CD172a) ligand, a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and a linker linking the first domain and the second domain. In embodiments, the anti-FOLR1 antibody is farletuzumab or mirvetuximab. In embodiments, the anti-FOLR1 antibody is mirvetuximab.

### Combination Therapies and Conjugation

In embodiments, the present disclosure provides for chimeric proteins and methods that further comprise administering an additional agent to a subject. In embodiments, the present disclosure pertains to co-administration and/or co-formulation. Any of the compositions disclosed herein may be co-formulated and/or co-administered.

In one aspect, the present disclosure provides a method for treating a cancer in a subject comprising: (i) administering to the subject a pharmaceutical composition comprising a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain; and (ii) administering to the subject a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof.

In one aspect, the present disclosure provides a method for treating a cancer in a subject comprising: (i) administering to the subject a pharmaceutical composition comprising a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain; wherein the subject has undergone or is undergoing treatment with a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof.

In one aspect, the present disclosure provides a method for treating a cancer in a subject comprising: (i) administering to the subject a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; wherein the subject has undergone or is undergoing treatment with a pharmaceutical composition comprising a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain.

In embodiments, a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure disclosed herein acts synergistically when co-administered with another agent and is administered at doses that are lower than the doses commonly employed when such agents are used as monotherapy. In embodiments, any agent referenced herein may be used in combination with any of the chimeric proteins disclosed herein.

In embodiments, a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure disclosed herein may be used in combination with any of the anti-cancer therapy disclosed herein.

In embodiments, a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure disclosed herein acts synergistically with each other. In embodiments, the chimeric protein, as disclosed herein, reduces the number of administrations of the co-administered second pharmaceutical composition.

In aspects and embodiments of the present disclosure, a patient in need of a cancer treatment comprising a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure, as disclosed herein, is or is predicted to be poorly responsive or is non-responsive to an immunotherapy, e.g., an anti-cancer immunotherapy, as disclosed herein. Moreover, in embodiments, a patient in need of an anti-cancer agent, as disclosed herein, is or may is predicted to be poorly responsive or non-responsive to an immune checkpoint immunotherapy. The immune checkpoint molecule may be selected from PD-1, PD-L1, PD-L2, ICOS, ICOSL, and CTLA-4. Moreover, in embodiments, a patient in need of an anti-cancer agent, as disclosed herein, is or may is predicted to be poorly responsive or non-responsive to an therapy directed to one or more of epidermal growth factor receptor (EGFR), human epidermal growth factor receptor 2 (Her2), and CD20.

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure (and/or additional agents) disclosed herein, include derivatives that are modified, i.e., by the covalent attachment of any type of molecule to the composition such that covalent attachment does not prevent the activity of the composition. For example, but not by way of limitation, derivatives include composition that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of tunicamycin, etc. Additionally, the derivative can contain one or more non-classical amino acids.

The anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure (and/or other anti-cancer therapy) disclosed herein may thus be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure disclosed herein, include derivatives that are modified, i.e., by the covalent attachment of any type of molecule to the composition such that covalent attachment does not prevent the activity of the composition. For example, but not by way of limitation, derivatives include composition that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of turicamycin, etc. Additionally, the derivative can contain one or more non-classical amino acids.

The anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure disclosed herein may thus be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

### Pharmaceutical Composition

The methods of the present disclosure include administering pharmaceutical compositions comprising a therapeutically effective amount of, at least one, second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure, as disclosed herein.

The anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure (and/or additional agents) disclosed herein can possess a sufficiently basic functional group, which can react with an inorganic or organic acid, or a carboxyl group, which can react with an inorganic or organic base, to form a pharmaceutically acceptable salt. A pharmaceutically-acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In embodiments, the compositions disclosed herein are in the form of a pharmaceutically acceptable salt.

Further, a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure (and/or additional agents) disclosed herein can be administered to a subject as a component of a composition, e.g., pharmaceutical composition, that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent disclosed herein, if desired, can also comprise minor amounts of wetting or emulsifying agents, or pH buffering agents.

In embodiments, the compositions, e.g., pharmaceutical compositions, disclosed herein are resuspended in a saline buffer (including, without limitation TBS, PBS, and the like).

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure may by conjugated and/or fused with another agent to extend half-life or otherwise improve pharmacodynamic and pharmacokinetic properties. In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In embodiments, each of the individual chimeric proteins is fused to one or more of the agents described in BioDrugs (2015) 29:215-239, the entire contents of which are hereby incorporated by reference.

The present disclosure includes anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure (and/or additional agents) in various formulations of pharmaceutical composition. The second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure (and/or additional agents) disclosed herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In embodiments, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the pharmaceutical compositions comprising the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure (and/or additional agents) can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The pharmaceutical compositions comprising the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure (and/or additional agents) of the present disclosure may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure (and/or additional agents) disclosed herein is formulated in accordance with routine procedures as a pharmaceutical composition adapted for a mode of administration disclosed herein.

In one aspect, the present disclosure provides a method for treating a cancer in a subject comprising: (i) administering to the subject a pharmaceutical composition comprising a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain; and (ii) administering to the subject a second pharmaceutical composition comprising azacitidine and/or venetoclax.

In one aspect, the present disclosure provides a method for treating a cancer in a subject comprising: (i) administering to the subject a pharmaceutical composition comprising a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain; wherein the subject has undergone or is undergoing treatment with a second pharmaceutical composition comprising azacitidine and/or venetoclax.

In one aspect, the present disclosure provides a method for treating a cancer in a subject comprising: (i) administering to the subject a second pharmaceutical composition comprising azacitidine and/or venetoclax; wherein the subject has undergone or is undergoing treatment with a pharmaceutical composition comprising a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain.

In one aspect, the present disclosure provides a method for treating a cancer in a subject comprising: (i) administering to the subject a first pharmaceutical composition comprising a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain; and (ii) administering to the subject a second pharmaceutical composition comprising azacitidine; and (iii) administering to the subject a third pharmaceutical composition comprising venetoclax. In embodiments, the first pharmaceutical composition and the second pharmaceutical composition are administered simultaneously. In embodiments, the first pharmaceutical composition and the third pharmaceutical composition are administered simultaneously. In embodiments, second pharmaceutical composition and the third pharmaceutical composition are administered simultaneously. In embodiments, the first pharmaceutical composition, the second pharmaceutical composition and the third pharmaceutical composition are administered simultaneously. In embodiments, the first pharmaceutical composition is administered after the second pharmaceutical composition and/or the third pharmaceutical composition is administered. In embodiments, the second pharmaceutical composition is administered after and/or the first pharmaceutical composition and/or the third pharmaceutical composition is administered. In embodiments, the third pharmaceutical composition is administered after and/or the first pharmaceutical composition and/or the second pharmaceutical composition is administered.

In one aspect, the present disclosure provides a method for treating a cancer in a subject comprising: (i) administering to the subject a first pharmaceutical composition comprising a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain, wherein the subject has undergone or is undergoing treatment with a second pharmaceutical composition comprising azacitidine and/or a third pharmaceutical composition comprising venetoclax. In embodiments, the subject has undergone or is undergoing treatment with the second pharmaceutical composition after the third pharmaceutical composition. In embodiments, the subject has undergone or is undergoing treatment with the third pharmaceutical composition after the second pharmaceutical composition.

In one aspect, the present disclosure provides a method for treating a cancer in a subject comprising: (i) administering to the subject a second pharmaceutical composition comprising azacitidine, wherein the subject has undergone or is undergoing treatment with a first pharmaceutical composition and/or a third pharmaceutical composition comprising venetoclax, wherein the first pharmaceutical composition comprises a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain. In embodiments, the subject has undergone or is undergoing treatment with the first pharmaceutical composition after the third pharmaceutical composition. In embodiments, the subject has undergone or is undergoing treatment with the third pharmaceutical composition after the first pharmaceutical composition.

In one aspect, the present disclosure provides a method for treating a cancer in a subject comprising: (i) administering to the subject a third pharmaceutical composition comprising venetoclax, wherein the subject has undergone or is undergoing treatment with a first pharmaceutical composition and/or a second pharmaceutical composition comprising azacitidine, wherein the first pharmaceutical composition comprises a heterologous chimeric protein comprising: (a) a first domain comprising a portion of the extracellular domain of SIRPα(CD172a), wherein the portion is capable of binding a SIRPα(CD172a) ligand, (b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and (c) a linker linking the first domain and the second domain. In embodiments, the subject has undergone or is undergoing treatment with the first pharmaceutical composition after the second pharmaceutical composition. In embodiments, the subject has undergone or is undergoing treatment with the second pharmaceutical composition after the first pharmaceutical composition.

### Administration, Dosing, and Treatment Regimens

Routes of administration include, for example: intradermal, intratumoral, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin.

As examples, administration results in the release of anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure (and/or additional agents) disclosed herein into the bloodstream (via enteral or parenteral administration), or alternatively, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure (and/or additional agents) is administered directly to the site of active disease.

The second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure (and/or additional agents) disclosed herein can be administered orally. Such anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure (and/or additional agents) can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

In specific embodiments, it may be desirable to administer locally to the area in need of treatment. In embodiments, for instance in the treatment of cancer, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure (and/or additional agents) are administered in the tumor microenvironment (e.g., cells, molecules, extracellular matrix and/or blood vessels that surround and/or feed a tumor cell, inclusive of, for example, tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor) or lymph node and/or targeted to the tumor microenvironment or lymph node. In embodiments, for instance in the treatment of cancer, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure (and/or additional agents) are administered intratumorally.

In embodiments, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure allows for a dual effect that provides less side effects than are seen in conventional immunotherapy (e.g., treatments with one or more of OPDIVO, KEYTRUDA, YERVOY, and TECENTRIQ). For example, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure reduce or prevent commonly observed immune-related adverse events that affect various tissues and organs including the skin, the gastrointestinal tract, the kidneys, peripheral and central nervous system, liver, lymph nodes, eyes, pancreas, and the endocrine system; such as hypophysitis, colitis, hepatitis, pneumonitis, rash, and rheumatic disease. Further, the present local administration, e.g., intratumorally, obviate adverse event seen with standard systemic administration, e.g., IV infusions, as are used with conventional immunotherapy (e.g., treatments with one or more of OPDIVO, KEYTRUDA, YERVOY, and TECENTRIQ).

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure (and/or additional agents) disclosed herein as well as the dosing schedule can depend on various parameters, including, but not limited to, the disease being treated, the subject's general health, and the administering physician's discretion. The second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure, disclosed herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional agent, to a subject in need thereof.

In embodiments, a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure and an additional agent(s) are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days apart, 4 days apart, 5 days apart, 6 days apart, 1 week apart, 2 weeks apart, 3 weeks apart, or 4 weeks apart.

In embodiments, the present disclosure relates to the co-administration of a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure which induces an innate immune response and another antibody directed to immune checkpoint molecules; and/or chimeric protein used in methods of the present disclosure which induces an adaptive immune response. In such embodiments, the second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure which induces an innate immune response may be administered before, concurrently with, or subsequent to administration of the second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure which induces an adaptive immune response. For example, the anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric proteins used in methods of the present disclosure may be administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days apart, 4 days apart, 5 days apart, 6 days apart, 1 week apart, 2 weeks apart, 3 weeks apart, or 4 weeks apart. In an illustrative embodiment, second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure which induces an innate immune response and the second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure which induces an adaptive response are administered 1 week apart, or administered on alternate weeks (i.e., administration of the second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure inducing an innate immune response is followed 1 week later with administration of the second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure which induces an adaptive immune response and so forth).

The dosage of a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure (and/or additional agents) disclosed herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

For administration of a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure (and/or additional agents) disclosed herein by parenteral injection, the dosage may be about 0.1 mg to about 250 mg per day, about 1 mg to about 20 mg per day, or about 3 mg to about 5 mg per day. Generally, when orally or parenterally administered, the dosage of any agent disclosed herein may be about 0.1 mg to about 1500 mg per day, or about 0.5 mg to about 10 mg per day, or about 0.5 mg to about 5 mg per day, or about 200 to about 1,200 mg per day (e.g., about 200 mg, about 300 mg, about 400 mg, about 500 mg, about 600 mg, about 700 mg, about 800 mg, about 900 mg, about 1,000 mg, about 1,100 mg, about 1,200 mg per day).

In embodiments, administration of the second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure (and/or additional agents) disclosed herein is by parenteral injection at a dosage of about 0.1 mg to about 1500 mg per treatment, or about 0.5 mg to about 10 mg per treatment, or about 0.5 mg to about 5 mg per treatment, or about 200 to about 1,200 mg per treatment (e.g., about 200 mg, about 300 mg, about 400 mg, about 500 mg, about 600 mg, about 700 mg, about 800 mg, about 900 mg, about 1,000 mg, about 1,100 mg, about 1,200 mg per treatment).

In embodiments, a suitable dosage of the second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure (and/or additional agents) is in a range of about 0.01 mg/kg to about 100 mg/kg of body weight or about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, inclusive of all values and ranges therebetween.

In another embodiment, delivery can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

An second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure (and/or additional agents) disclosed herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure (and/or additional agents) disclosed herein can, independently, be one to four times daily or one to four times per month or one to six times per year or once every two, three, four or five years. Administration can be for the duration of one day or one month, two months, three months, six months, one year, two years, three years, and may even be for the life of the subject.

The dosage regimen utilizing a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure (and/or additional agents) disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the present disclosure employed. The second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure (and/or additional agents) disclosed herein can be administered in a single daily dose, or the total daily dosage can be administered in divided doses of two, three or four times daily. Furthermore, a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure (and/or additional agents) disclosed herein can be administered continuously rather than intermittently throughout the dosage regimen.

### Fusion Proteins, Nucleic Acids, and Cells

A chimeric protein used in a method of the present disclosure may be a recombinant fusion protein, e.g., a single polypeptide having the extracellular domains disclosed herein. For example, in embodiments, the chimeric protein is translated as a single unit in a prokaryotic cell, a eukaryotic cell, or a cell-free expression system.

In embodiments, a chimeric protein is recombinant protein comprising multiple polypeptides, e.g., multiple extracellular domains disclosed herein, that are combined (via covalent or non-covalent bonding) to yield a single unit, e.g., in vitro (e.g., with one or more synthetic linkers disclosed herein).

In embodiments, a chimeric protein is chemically synthesized as one polypeptide or each domain may be chemically synthesized separately and then combined. In embodiments, a portion of the chimeric protein is translated and a portion is chemically synthesized.

Constructs could be produced by cloning of the nucleic acids encoding the three fragments (the extracellular domain of a Type I transmembrane protein, followed by a linker sequence, followed by the extracellular domain of a Type II transmembrane protein) into a vector (plasmid, viral or other) wherein the amino terminus of the complete sequence corresponded to the ‘left’ side of the molecule containing the extracellular domain of the Type I transmembrane protein and the carboxy terminus of the complete sequence corresponded to the ‘right’ side of the molecule containing the extracellular domain of Type II transmembrane protein. In embodiments, of chimeric proteins having one of the other configurations, as described elsewhere herein, a construct would comprise three nucleic acids such that the translated chimeric protein produced would have the desired configuration, e.g., a dual inward-facing chimeric protein. Accordingly, in embodiments, the chimeric proteins used in methods of the present disclosure are engineered as such.

A chimeric protein used in a method of the present disclosure may be encoded by a nucleic acid cloned into an expression vector. In embodiments, the expression vector comprises DNA or RNA. In embodiments, the expression vector is a mammalian expression vector.

Both prokaryotic and eukaryotic vectors can be used for expression of the chimeric protein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and λPL. Non-limiting examples of prokaryotic expression vectors may include the λgt vector series such as λgt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful. A variety of regulatory regions can be used for expression of the chimeric proteins in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the chimeric proteins in recombinant host cells.

In embodiments, expression vectors comprise a nucleic acid encoding the chimeric proteins, or a complement thereof, operably linked to an expression control region, or complement thereof, that is functional in a mammalian cell. The expression control region is capable of driving expression of the operably linked blocking and/or stimulating agent-encoding nucleic acid such that the blocking and/or stimulating agent is produced in a human cell transformed with the expression vector.

In embodiments, a chimeric protein used in a method of the present disclosure is producible in a mammalian host cell as a secretable and fully functional single polypeptide chain.

Expression control regions are regulatory polynucleotides (sometimes referred to herein as elements), such as promoters and enhancers, that influence expression of an operably linked nucleic acid. An expression control region of an expression vector of the present disclosure is capable of expressing operably linked encoding nucleic acid in a human cell. In embodiments, the cell is a tumor cell. In another embodiment, the cell is a non-tumor cell. In embodiments, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

In embodiments, the present disclosure contemplates the use of inducible promoters capable of effecting high level of expression transiently in response to a cue. For example, when in the proximity of a tumor cell, a cell transformed with an expression vector for the chimeric protein (and/or additional agents) comprising such an expression control sequence is induced to transiently produce a high level of the agent by exposing the transformed cell to an appropriate cue. Illustrative inducible expression control regions include those comprising an inducible promoter that is stimulated with a cue such as a small molecule chemical compound. In other examples, the chimeric protein is expressed by a chimeric antigen receptor containing cell or an in vitro expanded tumor infiltrating lymphocyte, under the control of a promoter which is sensitive to antigen recognition by the cell, and leads to local secretion of the chimeric protein in response to tumor antigen recognition. Particular examples can be found, for example, in U.S. Pat. Nos. 5,989,910, 5,935,934, 6,015,709, and 6,004,941, each of which is incorporated herein by reference in its entirety.

Expression control regions and locus control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

As used herein, “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Expression systems functional in human cells are well known in the art; these include viral systems. Generally, a promoter functional in a human cell is any DNA sequence capable of binding mammalian RNA polymerase and initiating the downstream (3′) transcription of a coding sequence into mRNA. A promoter will have a transcription-initiating region, which is usually placed proximal to the 5′ end of the coding sequence, and, typically, a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated, and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs.

There is a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a tumor cell surface membrane protein. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), Flp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences encoding the chimeric fusion proteins including CRISPR/CAS9, zinc finger, TALEN, and meganuclease gene-editing technologies.

In embodiments, the expression vectors for the expression of the chimeric proteins (and/or additional agents) are viral vectors. Many viral vectors useful for gene therapy are known (see, e.g., Lundstrom, Trends Biotechnol., 21: 1 17, 122, 2003. Illustrative viral vectors include those selected from antiviruses (LV), retroviruses (RV), adenoviruses (AV), adeno-associated viruses (AAV), and a viruses, though other viral vectors may also be used. For in vivo uses, viral vectors that do not integrate into the host genome are suitable for use, such as a viruses and adenoviruses. Illustrative types of a viruses include Sindbis virus, Venezuelan equine encephalitis (VEE) virus, and Semliki Forest virus (SFV). For in vitro uses, viral vectors that integrate into the host genome are suitable, such as retroviruses, AAV, and antiviruses. In embodiments, the present disclosure provides methods of transducing a human cell in vivo, comprising contacting a solid tumor in vivo with a viral vector of the present disclosure.

Expression vectors can be introduced into host cells for producing the chimeric proteins used in methods of the present disclosure. Cells may be cultured in vitro or genetically engineered, for example. Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al., J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); DG44 CHO cells, CHO-K1 cells, mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the chimeric proteins disclosed herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, and human small cell lung carcinoma cell lines, SCLC #2 and SCLC #7.

Host cells can be obtained from normal or affected subjects, including healthy humans, cancer patients, and patients with an infectious disease, private laboratory deposits, public culture collections such as the American Type Culture Collection (ATCC), or from commercial suppliers.

Cells that can be used for production of the chimeric proteins used in methods of the present disclosure in vitro, ex vivo, and/or in vivo include, without limitation, epithelial cells, endothelial cells, keratinocytes, fibroblasts, muscle cells, hepatocytes; blood cells such as T lymphocytes, chimeric antigen receptor expressing T cells, tumor infiltrating lymphocytes, B lymphocytes, monocytes, macrophages, neutrophils, eosinophils, megakaryocytes, granulocytes; various stem or progenitor cells, in particular hematopoietic stem or progenitor cells (e.g., as obtained from bone marrow), umbilical cord blood, peripheral blood, and fetal liver. The choice of cell type depends on the type of tumor or infectious disease being treated or prevented, and can be determined by one of skill in the art.

Production and purification of Fc-containing macromolecules (such as monoclonal antibodies) has become a standardized process, with minor modifications between products. For example, many Fc containing macromolecules are produced by human embryonic kidney (HEK) cells (or variants thereof) or Chinese Hamster Ovary (CHO) cells (or variants thereof) or in some cases by bacterial or synthetic methods. Following production, the Fc containing macromolecules that are secreted by HEK or CHO cells are purified through binding to Protein A columns and subsequently ‘polished’ using various methods. Generally speaking, purified Fc containing macromolecules are stored in liquid form for some period of time, frozen for extended periods of time or in some cases lyophilized. In embodiments, production of the chimeric proteins contemplated herein may have unique characteristics as compared to traditional Fc containing macromolecules. In certain examples, the chimeric proteins may be purified using specific chromatography resins, or using chromatography methods that do not depend upon Protein A capture. In embodiments, the chimeric proteins may be purified in an oligomeric state, or in multiple oligomeric states, and enriched for a specific oligomeric state using specific methods. Without being bound by theory, these methods could include treatment with specific buffers including specified salt concentrations, pH and additive compositions. In other examples, such methods could include treatments that favor one oligomeric state over another. The chimeric proteins obtained herein may be additionally ‘polished’ using methods that are specified in the art. In embodiments, the chimeric proteins are highly stable and able to tolerate a wide range of pH exposure (between pH 3-12), are able to tolerate a large number of freeze/thaw stresses (greater than 3 freeze/thaw cycles) and are able to tolerate extended incubation at high temperatures (longer than 2 weeks at 40 degrees C.). In embodiments, the chimeric proteins are shown to remain intact, without evidence of degradation, deamidation, etc. under such stress conditions.

Subjects and/or Animals

In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In embodiments, the subject and/or animal is a non-mammal, such, for example, a zebrafish. In embodiments, the subject and/or animal may comprise fluorescently tagged cells (with e.g., GFP). In embodiments, the subject and/or animal is a transgenic animal, which comprises a fluorescent cell.

In embodiments, the subject and/or animal is a human. In embodiments, the human is a pediatric human. In embodiments, the human is an adult human. In embodiments, the human is a geriatric human. In embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In embodiments, the subject is a non-human animal, and therefore the present disclosure pertains to veterinary use. In a specific embodiment, the non-human animal is a household pet. In another specific embodiment, the non-human animal is a livestock animal.

In embodiments, the subject has a cancer that is poorly responsive or is refractory to treatment comprising an antibody that is capable of binding PD-1 or binding a PD-1 ligand. In embodiments, the subject has a cancer that is poorly responsive or is non-responsive to treatment with an antibody that is capable of binding PD-1 or binding a PD-1 ligand after 12 weeks or so of such treatment.

### Kits and Medicaments

Aspects of the present disclosure provide kits that can simplify the administration of the pharmaceutical compositions and/or chimeric proteins disclosed herein.

An illustrative kit of the present disclosure comprises any anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof; and/or chimeric protein used in methods of the present disclosure and/or pharmaceutical composition disclosed herein in unit dosage form. In embodiments, the unit dosage form is a container, such as a pre-filled syringe, which can be sterile, containing any agent disclosed herein and a pharmaceutically acceptable carrier, diluent, excipient, or vehicle. The kit can further comprise a label or printed instructions instructing the use of any agent disclosed herein. The kit may also include a lid speculum, topical anesthetic, and a cleaning agent for the administration location. The kit can also further comprise one or more additional agent disclosed herein. In embodiments, the kit comprises a container containing an effective amount of a composition of the present disclosure and an effective amount of another composition, such those disclosed herein.

Aspects of the present disclosure include use of a chimeric protein as disclosed herein in the manufacture of a medicament, e.g., a medicament for treatment of cancer.

Any aspect or embodiment disclosed herein can be combined with any other aspect or embodiment as disclosed herein.

The present disclosure will be further described in the following examples, which do not limit the scope of the present disclosure described in the claims.

### EXAMPLES

The examples herein are provided to illustrate advantages and benefits of the present disclosure and to further assist a person of ordinary skill in the art with preparing or using the chimeric proteins of the present disclosure. The examples herein are also presented in order to more fully illustrate the preferred aspects of the present disclosure. The examples should in no way be construed as limiting the scope of the present disclosure, as defined by the appended claims. The examples can include or incorporate any of the variations, aspects or embodiments of the present disclosure described above. The variations, aspects or embodiments described above may also further each include or incorporate the variations of any or all other variations, aspects or embodiments of the present disclosure.

### Example 1: Amplification of the Phagocytosis-Stimulating Activity of the SIRPα-Fc-CD40L Chimeric Protein by a Hypomethylating Agent/an Epigenetic Regulator

The effect of the treatment of cancer cells by a hypomethylating agent/epigenetic regulator on the phagocytosis-stimulating activity of the SIRPα-Fc-CD40L chimeric protein was determined.

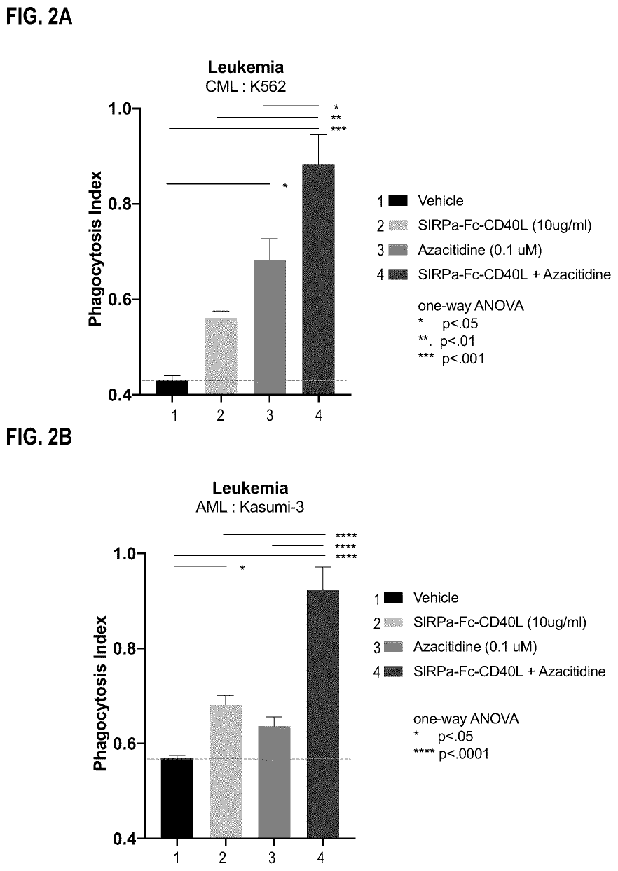

Briefly, the K652 human chronic myelogenous leukemia (CML) cells were labeled with a green fluorescent tracker and treated with vehicle alone control or 0.1 μM azacitidine overnight. The following day, the tumor cells were washed in PBS and then co-cultured with human macrophages with or without the SIRPα-Fc-CD40L chimeric protein for 4 hours at 37° C. in the presence of 5% CO₂. After this incubation, the cells were harvested and treated with an anti-CD11b antibody (a macrophage marker) and were analyzed using flow cytometry. Positive phagocytosis was determined by the overlap in signals of tumor (the green fluorescent tracker) and macrophage (anti-CD11b antibody staining). A phagocytosis index was calculated by setting the maximum phagocytosis value to 1, and then normalizing all other replicates accordingly. The phagocytosis index was plotted for the indicated treatments. As shown in FIG. 2A, the K652 cells that were treated with vehicle alone control overnight and then co-cultured with human macrophages in the absence of the SIRPα-Fc-CD40L chimeric protein (the control K652 cells), were phagocytized by the human macrophages at a background level. The treatment with azacitidine (overnight) alone or the SIRPα-Fc-CD40L chimeric protein (for 4 hours) alone resulted in an increased level of phagocytosis compared to the control K652 cells (FIG. 2A). Interestingly, as shown in in FIG. 2A, the K652 cells that were treated azacitidine (overnight) and the SIRPα-Fc-CD40L chimeric protein (for 4 hours), exhibited increased level of phagocytosis, which was statistically significant over the control K652 cells (p<0.001), the K652 cells treated with azacitidine alone (overnight) (p<0.05), or the K652 cells treated with the SIRPα-Fc-CD40L chimeric protein alone (for 4 hours) (p<0.01).

In another experiment, the Kasumi-3 human acute myelocytic leukemia (AML) cells were labeled with a green fluorescent tracker and treated with vehicle alone control or 0.1 pM azacitidine overnight. The following day, the tumor cells were washed in PBS and then co-cultured with human macrophages with or without the SIRPα-Fc-CD40L chimeric protein for 4 hours at 37° C. in the presence of 5% CO₂. After this incubation, the cells were harvested and treated with an anti-CD11b antibody (a macrophage marker) and were analyzed using flow cytometry. Positive phagocytosis was determined by the overlap in signals of tumor (the green fluorescent tracker) and macrophage (anti-CD11b antibody staining). A phagocytosis index was calculated by setting the maximum phagocytosis value to 1, and then normalizing all other replicates accordingly. The phagocytosis index was plotted for the indicated treatments. As shown in FIG. 2B, the Kasumi-3 cells that were treated with vehicle alone control overnight and then co-cultured with human macrophages in the absence of the SIRPα-Fc-CD40L chimeric protein (the control Kasumi-3 cells), were phagocytized by the human macrophages at a background level. The treatment with azacitidine (overnight) alone or the SIRPα-Fc-CD40L chimeric protein (for 4 hours) alone resulted in a slight increase in the level of phagocytosis compared to the control Kasumi-3 cells (FIG. 2B). Interestingly, as shown in in FIG. 2B, the Kasumi-3 cells that were treated azacitidine (overnight) and the SIRPα-Fc-CD40L chimeric protein (for 4 hours), exhibited increased level of phagocytosis, which was statistically significant over the control Kasumi-3 cells (p<0.0001), the Kasumi-3 cells treated with azacitidine alone (overnight) (p<0.0001), or the Kasumi-3 cells treated with the SIRPα-Fc-CD40L chimeric protein alone (for 4 hours) (p<0.0001).

Collectively, these results show that the combination of SIRPα-Fc-CD40L with hypomethylation agents such as azacitidine, enhance the phagocytosis of hematological tumors such as CML and AML. These results demonstrate that the hypomethylating agent azacitidine potentiates the phagocytosis-stimulating activity of the SIRPα-Fc-CD40L chimeric protein. Therefore, these results indicate that a combination therapy of cancer with the SIRPα-Fc-CD40L chimeric protein and a hypomethylating agent/an epigenetic regulator is likely to produce a superior efficacy compared to both the SIRPα-Fc-CD40L chimeric protein and the hypomethylating agent/epigenetic regulator.

### Example 2: Amplification of the Phagocytosis-Stimulating Activity of the SIRPα-Fc-CD40L Chimeric Protein by a Proteasomal Inhibitor

The effect of the treatment of cancer cells by a proteasomal inhibitor on the phagocytosis-stimulating activity of the SIRPα-Fc-CD40L chimeric protein was determined.

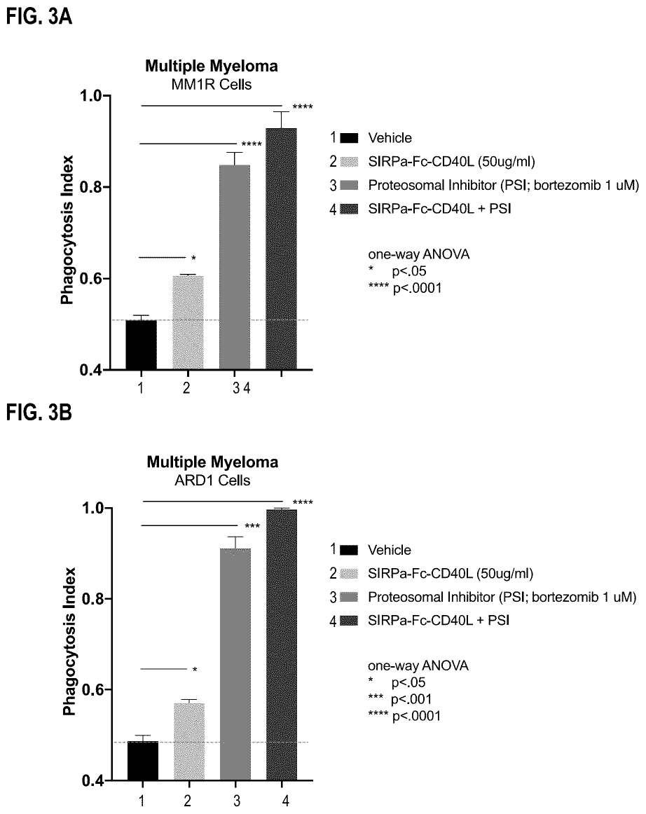

Briefly, the MM1R human multiple myeloma (MM) cells were labeled with a green fluorescent tracker and treated with vehicle alone control or 1 pM bortezomib overnight. The following day, the tumor cells were washed in PBS and then co-cultured with human macrophages with or without the SIRPα-Fc-CD40L chimeric protein for 4 hours at 37° C. in the presence of 5% CO₂. After this incubation, the cells were harvested and treated with an anti-CD11b antibody (a macrophage marker) and were analyzed using flow cytometry. Positive phagocytosis was determined by the overlap in signals of tumor (the green fluorescent tracker) and macrophage (anti-CD11b antibody staining). A phagocytosis index was calculated by setting the maximum phagocytosis value to 1, and then normalizing all other replicates accordingly. The phagocytosis index was plotted for the indicated treatments. As shown in FIG. 3A, the MM1R cells that were treated with vehicle alone control overnight and then co-cultured with human macrophages in the absence of the SIRPα-Fc-CD40L chimeric protein (the control MM1R cells), were phagocytized by the human macrophages at a background level. The treatment with bortezomib (overnight) alone or the SIRPα-Fc-CD40L chimeric protein (for 4 hours) alone resulted in an increased level of phagocytosis compared to the control MM1R cells (FIG. 3A). Interestingly, as shown in in FIG. 3A, the MM1R cells that were treated bortezomib (overnight) and the SIRPα-Fc-CD40L chimeric protein (for 4 hours), exhibited increased level of phagocytosis compared to the control MM1R cells (p<0.0001), the MM1R cells treated with bortezomib alone (overnight), or the MM1R cells treated with the SIRPα-Fc-CD40L chimeric protein alone (for 4 hours).

In another experiment, the ARD1 human multiple myeloma (MM) cells were labeled with a green fluorescent tracker and treated with vehicle alone control or 1 pM bortezomib overnight. The following day, the tumor cells were washed in PBS and then co-cultured with human macrophages with or without the SIRPα-Fc-CD40L chimeric protein for 4 hours at 37° C. in the presence of 5% CO₂. After this incubation, the cells were harvested and treated with an anti-CD11b antibody (a macrophage marker) and were analyzed using flow cytometry. Positive phagocytosis was determined by the overlap in signals of tumor (the green fluorescent tracker) and macrophage (anti-CD11b antibody staining). A phagocytosis index was calculated by setting the maximum phagocytosis value to 1, and then normalizing all other replicates accordingly. The phagocytosis index was plotted for the indicated treatments. As shown in FIG. 3B, the ARD1 cells that were treated with vehicle alone control overnight and then co-cultured with human macrophages in the absence of the SIRPα-Fc-CD40L chimeric protein (the control ARD1 cells), were phagocytized by the human macrophages at a background level. The treatment with bortezomib (overnight) alone or the SIRPα-Fc-CD40L chimeric protein (for 4 hours) alone resulted in an increased level of phagocytosis compared to the control ARD1 cells (FIG. 3B). Interestingly, as shown in in FIG. 3B, the ARD1 cells that were treated bortezomib (overnight) and the SIRPα-Fc-CD40L chimeric protein (for 4 hours), exhibited increased level of phagocytosis compared to the control ARD1 cells (p<0.0001), the ARD1 cells treated with bortezomib alone (overnight), or the ARD1 cells treated with the SIRPα-Fc-CD40L chimeric protein alone (for 4 hours).

These results demonstrate that the proteasomal inhibitor bortezomib potentiates the phagocytosis-stimulating activity of the SIRPα-Fc-CD40L chimeric protein. Therefore, these results indicate that a combination therapy of cancer with the SIRPα-Fc-CD40L chimeric protein and a proteasomal inhibitor is likely to produce a superior efficacy compared to both the SIRPα-Fc-CD40L chimeric protein and the proteasomal inhibitor.

### Example 3: Amplification of the Phagocytosis-Stimulating Activity of the SIRPα-Fc-CD40L Chimeric Protein by a Bcl2 Inhibitor

The effect of the treatment of cancer cells by a Bcl2 Inhibitor on the phagocytosis-stimulating activity of the SIRPα-Fc-CD40L chimeric protein was determined.

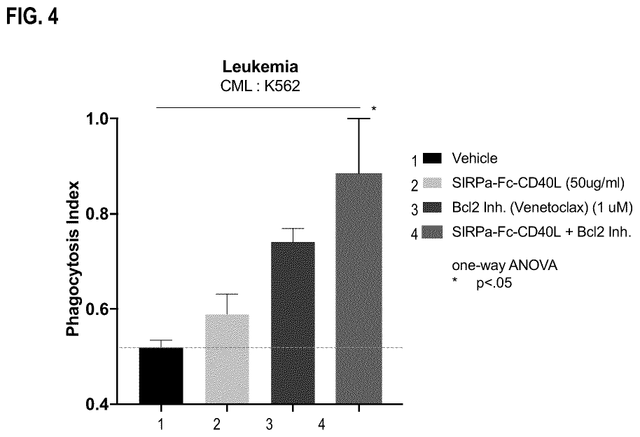

In another experiment, the K652 human chronic myelogenous leukemia (CML) cells were labeled with a green fluorescent tracker and treated with vehicle alone control or 1 μM venetoclax overnight. The following day, the tumor cells were washed in PBS and then co-cultured with human macrophages with or without the SIRPα-Fc-CD40L chimeric protein for 4 hours at 37° C. in the presence of 5% CO₂. After this incubation, the cells were harvested and treated with an anti-CD11b antibody (a macrophage marker) and were analyzed using flow cytometry. Positive phagocytosis was determined by the overlap in signals of tumor (the green fluorescent tracker) and macrophage (anti-CD11b antibody staining). A phagocytosis index was calculated by setting the maximum phagocytosis value to 1, and then normalizing all other replicates accordingly. The phagocytosis index was plotted for the indicated treatments. As shown in FIG. 4, the K562 cells that were treated with vehicle alone control overnight and then co-cultured with human macrophages in the absence of the SIRPα-Fc-CD40L chimeric protein (the control K562 cells), were phagocytized by the human macrophages at a background level. The treatment with venetoclax (overnight) alone or the SIRPα-Fc-CD40L chimeric protein (for 4 hours) alone resulted in an increased level of phagocytosis compared to the control K562 cells (FIG. 4). Interestingly, as shown in in FIG. 4, the K562 cells that were treated venetoclax (overnight) and the SIRPα-Fc-CD40L chimeric protein (for 4 hours), exhibited increased level of phagocytosis compared to the control K562 cells (p<0.0001), the K562 cells treated with venetoclax alone (overnight), or the K562 cells treated with the SIRPα-Fc-CD40L chimeric protein alone (for 4 hours).

These results demonstrate that the Bcl2 Inhibitor venetoclax potentiates the phagocytosis-stimulating activity of the SIRPα-Fc-CD40L chimeric protein. Therefore, these results indicate that a combination therapy of cancer with the SIRPα-Fc-CD40L chimeric protein and a Bcl2 Inhibitor is likely to produce a superior efficacy compared to both the SIRPα-Fc-CD40L chimeric protein and the Bcl2 Inhibitor.

### Example 4: Amplification of the Phagocytosis-Stimulating Activity of the SIRPα-Fc-CD40L Chimeric Protein by an Anti-BCMA Antibody

The effect of the treatment of cancer cells by an anti-BCMA antibody (clone C12A3.2), which has an antibody-dependent cellular phagocytosis (ADCP) activity, on the phagocytosis-stimulating activity of the SIRPα-Fc-CD40L chimeric protein was determined.

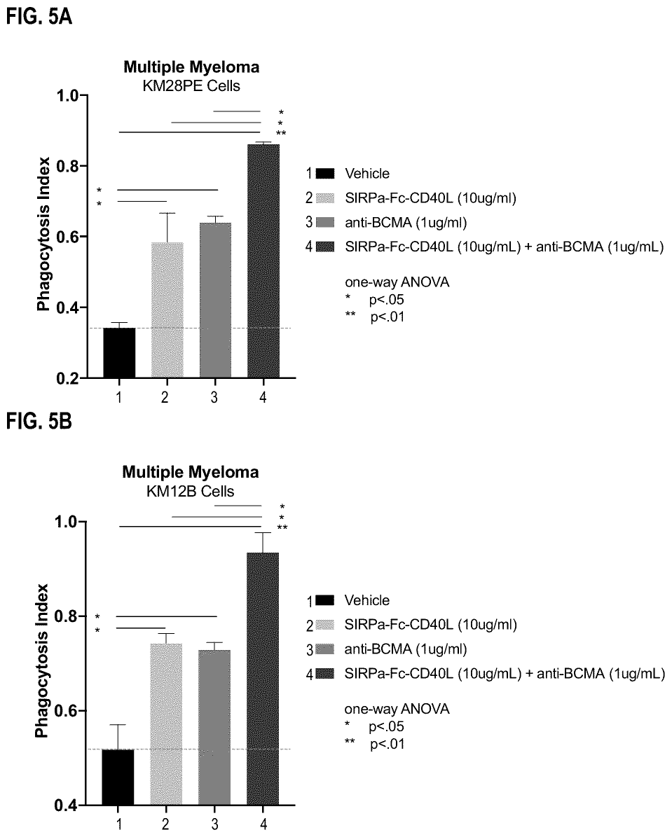

Briefly, the KM28PE human multiple myeloma (MM) cells were labeled with a green fluorescent tracker and co-cultured with human macrophages and treated with (1) vehicle alone control, (2) 10 μg/ml of the SIRPα-Fc-CD40L chimeric protein, (3) 1 μg/ml of an anti-BCMA antibody, or (4) 1 μg/ml of an anti-BCMA antibody and 10 μg/ml of the SIRPα-Fc-CD40L chimeric protein and incubated at 37° C. in the presence of 5% CO₂ for 4 hours. After the incubation, the cells were harvested and treated with an anti-CD11b antibody (a macrophage marker) and were analyzed using flow cytometry. Positive phagocytosis was determined by the overlap in signals of tumor (the green fluorescent tracker) and macrophage (anti-CD11b antibody staining). A phagocytosis index was calculated by setting the maximum phagocytosis value to 1, and then normalizing all other replicates accordingly. The phagocytosis index was plotted for the indicated treatments. As shown in FIG. 5A, the KM28PE cells that were treated with vehicle alone control were phagocytized by the human macrophages at a background level. The treatment with the anti-BCMA antibody alone or the SIRPα-Fc-CD40L chimeric protein alone resulted in an increased level of phagocytosis compared to the vehicle only-treated KM28PE cells (FIG. 5A). Interestingly, as shown in in FIG. 5A, the KM28PE cells treated the anti-BCMA antibody and the SIRPα-Fc-CD40L chimeric protein, exhibited increased level of phagocytosis compared to the vehicle only-treated KM28PE cells (p<0.01), the KM28PE cells treated with the anti-BCMA antibody alone (p<0.05), or the KM28PE cells treated with the SIRPα-Fc-CD40L chimeric protein alone (p<0.05).

In another experiment, the KM12B human multiple myeloma (MM) cells were labeled with a green fluorescent tracker and co-cultured with human macrophages and treated with (1) vehicle alone control, (2) 10 μg/ml of the SIRPα-Fc-CD40L chimeric protein, (3) 1 μg/ml of an anti-BCMA antibody, or (4) 1 μg/ml of an anti-BCMA antibody and 10 μg/ml of the SIRPα-Fc-CD40L chimeric protein and incubated at 37° C. in the presence of 5% CO₂ for 4 hours. After the incubation, the cells were harvested and treated with an anti-CD11b antibody (a macrophage marker) and were analyzed using flow cytometry. Positive phagocytosis was determined by the overlap in signals of tumor (the green fluorescent tracker) and macrophage (anti-CD11b antibody staining). A phagocytosis index was calculated by setting the maximum phagocytosis value to 1, and then normalizing all other replicates accordingly. The phagocytosis index was plotted for the indicated treatments. As shown in FIG. 5B, the KM12B cells that were treated with vehicle alone control were phagocytized by the human macrophages at a background level. The treatment with the anti-BCMA antibody alone or the SIRPα-Fc-CD40L chimeric protein alone resulted in an increased level of phagocytosis compared to the vehicle only-treated KM12B cells (FIG. 5B). Interestingly, as shown in in FIG. 5B, the KM12B cells treated the anti-BCMA antibody and the SIRPα-Fc-CD40L chimeric protein, exhibited increased level of phagocytosis compared to the vehicle only-treated KM12B cells (p<0.01), the KM12B cells treated with the anti-BCMA antibody alone (p<0.05), or the KM12B cells treated with the SIRPα-Fc-CD40L chimeric protein alone (p<0.05).

These results demonstrate that the anti-BCMA antibody potentiates the phagocytosis-stimulating activity of the SIRPα-Fc-CD40L chimeric protein. Therefore, these results indicate that a combination therapy of cancer with the SIRPα-Fc-CD40L chimeric protein and an anti-BCMA antibody is likely to produce a superior efficacy compared to both the SIRPα-Fc-CD40L chimeric protein and the anti-BCMA antibody. These data also suggest that a combination of the SIRPα-Fc-CD40L chimeric protein with ADCC/ADCP competent antibodies against tumor specific antigen targets (without limitation, e.g., PD-L1, CD47, CD38, FOLR1, CD123, SLAMF7 and BCMA) is likely to produce a superior efficacy compared to both the SIRPα-Fc-CD40L chimeric protein and the antibodies themselves.

### Example 5: Amplification of the Phagocytosis-Stimulating Activity of the SIRPα-Fc-CD40L Chimeric Protein by an Anti-CD38 Antibody

The effect of the treatment of cancer cells by an anti-CD38 antibody on the phagocytosis-stimulating activity of the SIRPα-Fc-CD40L chimeric protein was determined.

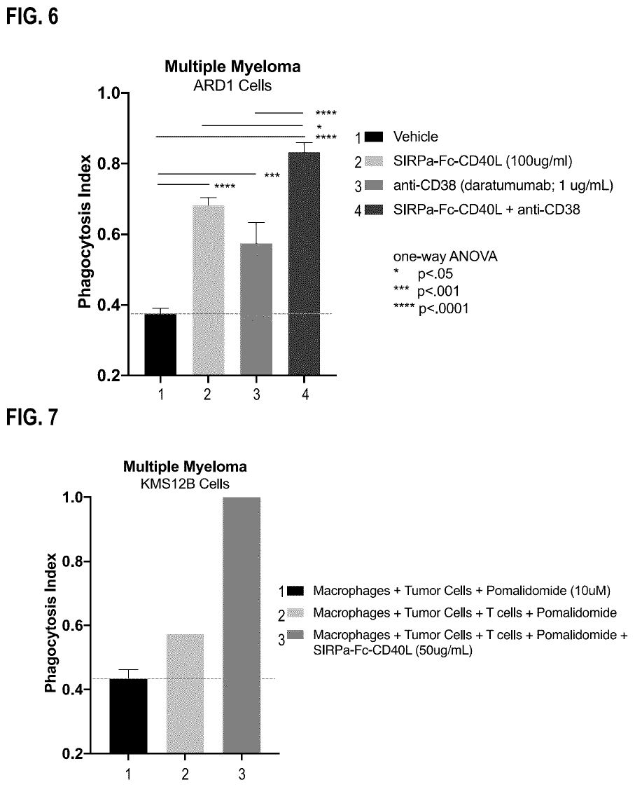

Briefly, the ARD1 human multiple myeloma (MM) cells were labeled the IncuCyte phRodo Red cell labeling kit and co-cultured with human macrophages and treated with (1) vehicle alone control, (2) 10 μg/ml of the SIRPα-Fc-CD40L chimeric protein, (3) 1 μg/ml of daratumumab (an antibody-dependent cellular phagocytosis (ADCP)-proficient anti-CD38 antibody), or (4) 1 μg/ml of an daratumumab and 10 μg/ml of the SIRPα-Fc-CD40L chimeric protein and incubated at 37° C. in the presence of 5% CO₂ for 2 hours. Cultures were imaged using the IncuCyte time-lapse microscopy system, and positive phagocytosis was determined by an increase in red fluorescent intensity which occurs when the phRodo Red labeled tumor cell is internalized into the acidic macrophage phagosome. A phagocytosis index was calculated by setting the maximum phagocytosis value to 1, and then normalizing all other replicates accordingly. The phagocytosis index was plotted for the indicated treatments. As shown in FIG. 6, the ARD1 cells that were treated with vehicle alone control were phagocytized by the human macrophages at a background level. The treatment with daratumumab alone or the SIRPα-Fc-CD40L chimeric protein alone resulted in an increased level of phagocytosis compared to the vehicle only-treated ARD1 cells (FIG. 6). Interestingly, as shown in in FIG. 6, the ARD1 cells treated daratumumab and the SIRPα-Fc-CD40L chimeric protein, exhibited increased level of phagocytosis compared to the vehicle only-treated ARD1 cells (p<0.0001), the ARD1 cells treated with daratumumab alone (p<0.0001), or the ARD1 cells treated with the SIRPα-Fc-CD40L chimeric protein alone (p<0.05).

These results demonstrate that the anti-CD38 antibody daratumumab potentiates the phagocytosis-stimulating activity of the SIRPα-Fc-CD40L chimeric protein. Therefore, these results indicate that a combination therapy of cancer with the SIRPα-Fc-CD40L chimeric protein and an anti-CD38 antibody is likely to produce a superior efficacy compared to both the SIRPα-Fc-CD40L chimeric protein and the anti-CD38 antibody. These data also suggest that a combination of the SIRPα-Fc-CD40L chimeric protein with ADCC/ADCP competent antibodies against tumor specific antigen targets (without limitation, e.g., PD-L1, CD47, CD38, FOLR1, CD123, SLAMF7 and BCMA) is likely to produce a superior efficacy compared to both the SIRPα-Fc-CD40L chimeric protein and the antibodies themselves.

### Example 6: Amplification of the Phagocytosis-Stimulating Activity of the SIRPα-Fc-CD40L Chimeric Protein by Immunomodulatory Imide Drugs (IMiDs)

The effect of the treatment of cancer cells by immunomodulatory imide drugs (IMiDs) on the phagocytosis-stimulating activity of the SIRPα-Fc-CD40L chimeric protein was determined.

Briefly, CD14+ monocytes were isolated from human donor PBMCs and cultured with m-CSF (100 ng/ml) for 6 days. On day 6, IFNγ (100 ng/ml) and LPS (10 ng/ml) were added to the cells for an additional 24 hours, generating M1 polarized macrophages. On day 5 of the macrophage differentiation, another vial of PBMCs from the same human donor was thawed, and CD3 T cells were isolated using a magnetic bead isolation kit. These T cells were activated for 2 days with CD3/CD28 T cell magnetic activation beads. On day 7 when both the macrophages and T cells were activated and ready, they were combined with KMS12B multiple myeloma cells, which were labeled with a green fluorescent tracker, with 10 μM pomalidomide and with or without 50 μg/ml of the SIPRα-Fc-CD40L chimeric protein. KMS12B cells incubated with the macrophages and 10 μM pomalidomide, without T cells, were used as a negative control. This coculture was incubated for 4 hours at 37° C., 5% CO₂. After this incubation, the cells were harvested and treated with an anti-CD11b antibody (a macrophage marker) and were analyzed using flow cytometry. Positive phagocytosis was determined by the overlap in signals of tumor (the green fluorescent tracker) and macrophage (anti-CD11b antibody staining). A phagocytosis index was calculated by setting the maximum phagocytosis value to 1, and then normalizing all other replicates accordingly. The phagocytosis index was plotted for the indicated treatments. When macrophages and tumor cells are combined in the presence of pomalidomide, a baseline phagocytosis signal was generated (black bar; FIG. 7). As shown in FIG. 7, the KMS12B cells that were incubated with T cells, macrophages and pomalidomide showed increased phagocytosis compared to the KMS12B cells that were incubated without T cells (the negative control). Further, as shown in in FIG. 7, the KMS12B cells treated pomalidomide and the SIRPα-Fc-CD40L chimeric protein exhibited further phagocytosis compared to the KMS12B cells treated without the SIPRα-Fc-CD40L chimeric protein.

IMiDs such as pomalidomide have been shown to modulate immune cells and enhance effector function. Without being bound by theory, it is likely that when CD3/CD28 activated T cells are also present, phagocytosis is increased, potentially due to the cytotoxic effect that the T cells have on the tumor cells, making them better targets for macrophage mediated phagocytosis. The addition of SIRPα-Fc-CD40L to this system, potentiated phagocytosis further. Therefore, these data demonstrate that the combination of an immune cell activator with an agent that enhances phagocytosis appear to synergize well. Therefore, these results indicate that a combination therapy of cancer with the SIRPα-Fc-CD40L chimeric protein and one of the IMiDs is likely to produce a superior efficacy compared to both the SIRPα-Fc-CD40L chimeric protein and the anti-CD38 antibody.

### Example 7: Amplification of the Phagocytosis-Stimulating Activity of the SIRPα-Fc-CD40L Chimeric Protein by an Anti-SLAMF7 Antibody

The effect of the treatment of cancer cells by an anti-SLAMF7 antibody on the phagocytosis-stimulating activity of the SIRPα-Fc-CD40L chimeric protein was determined.

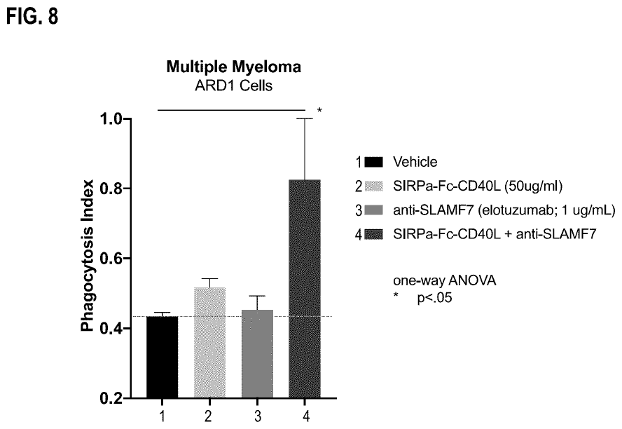

Briefly, the ARD1 human multiple myeloma cells were labeled with a green fluorescent tracker and co-cultured with human macrophages and treated with (1) vehicle alone control, (2) 10 μg/ml of the SIRPα-Fc-CD40L chimeric protein, (3) 1 μg/ml of elotuzumab (an antibody-dependent cellular phagocytosis (ADCP)-proficient anti-SLAMF7 antibody), or (4) 1 μg/ml of elotuzumab and 10 pg/ml of the SIRPα-Fc-CD40L chimeric protein and incubated at 37° C. in the presence of 5% CO₂ for 4 hours. After this incubation, the cells were harvested and treated with an anti-CD11b antibody (a macrophage marker) and were analyzed using flow cytometry. Positive phagocytosis was determined by the overlap in signals of tumor (the green fluorescent tracker) and macrophage (anti-CD11b antibody staining). A phagocytosis index was calculated by setting the maximum phagocytosis value to 1, and then normalizing all other replicates accordingly. The phagocytosis index was plotted for the indicated treatments. As shown in FIG. 8, the ARD1 cells that were treated with vehicle alone control were phagocytized by the human macrophages at a background level. The treatment with elotuzumab alone or the SIRPα-Fc-CD40L chimeric protein alone resulted in an increased level of phagocytosis compared to the vehicle only-treated ARD1 cells (FIG. 8). Interestingly, as shown in in FIG. 8, the ARD1 cells treated elotuzumab and the SIRPα-Fc-CD40L chimeric protein, exhibited increased level of phagocytosis compared to the vehicle only-treated ARD1 cells (p<0.05), the ARD1 cells treated with elotuzumab alone, or the ARD1 cells treated with the SIRPα-Fc-CD40L chimeric protein alone.

These results demonstrate that the anti-SLAMF7 antibody elotumab potentiates the phagocytosis-stimulating activity of the SIRPα-Fc-CD40L chimeric protein. Therefore, these results indicate that a combination therapy of cancer with the SIRPα-Fc-CD40L chimeric protein and an anti-SLAMF7 antibody is likely to produce a superior efficacy compared to both the SIRPα-Fc-CD40L chimeric protein and the anti-SLAMF7 antibody. These data also suggest that a combination of the SIRPα-Fc-CD40L chimeric protein with ADCC/ADCP competent antibodies against tumor specific antigen targets (without limitation, e.g., PD-L1, CD47, CD38, FOLR1, CD123, SLAMF7 and BCMA) is likely to produce a superior efficacy compared to both the SIRPα-Fc-CD40L chimeric protein and the antibodies themselves.

### Example 8: Amplification of the Phagocytosis-Stimulating Activity of the SIRPα-Fc-CD40L Chimeric Protein by an Anti-FOLR1 Antibody

The effect of the treatment of cancer cells by an anti-FOLR1 antibody on the phagocytosis-stimulating activity of the SIRPα-Fc-CD40L chimeric protein was determined.

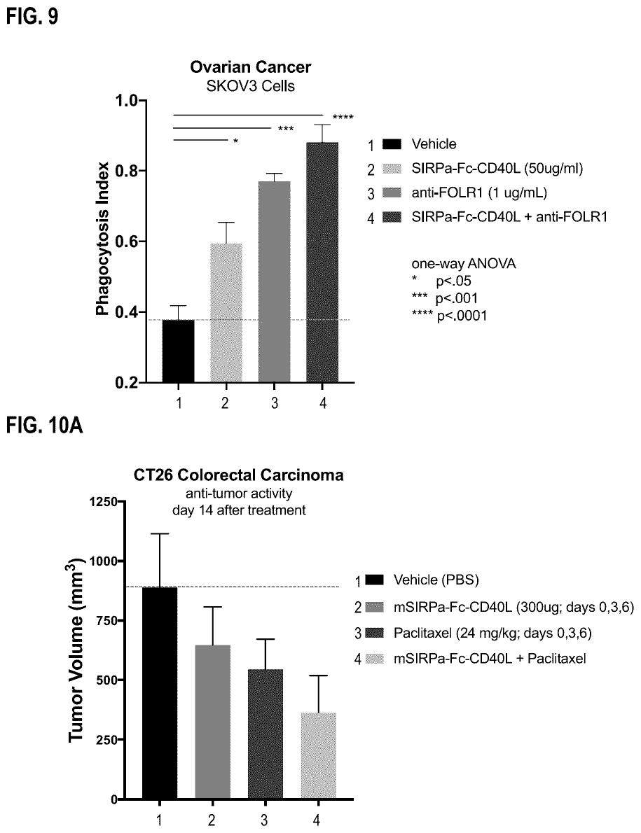

Briefly, the SKOV3 ovarian cancer cells were labeled with a green fluorescent tracker and co-cultured with human macrophages and treated with (1) vehicle alone control, (2) 10 μg/ml of the SIRPα-Fc-CD40L chimeric protein, (3) 1 μg/ml of an anti-FOLR1 antibody, or (4) 1 μg/ml of the anti-FOLR1 antibody and 10 μg/ml of the SIRPα-Fc-CD40L chimeric protein and incubated at 37° C. in the presence of 5% CO₂ for 4 hours. After this incubation, the cells were harvested and treated with an anti-CD11b antibody (a macrophage marker) and were analyzed using flow cytometry. Positive phagocytosis was determined by the overlap in signals of tumor (the green fluorescent tracker) and macrophage (anti-CD11b antibody staining). A phagocytosis index was calculated by setting the maximum phagocytosis value to 1, and then normalizing all other replicates accordingly. The phagocytosis index was plotted for the indicated treatments. As shown in FIG. 9, the SKOV3 cells that were treated with vehicle alone control were phagocytized by the human macrophages at a background level. The treatment with the anti-FOLR1 antibody alone or the SIRPα-Fc-CD40L chimeric protein alone resulted in an increased level of phagocytosis compared to the vehicle only-treated SKOV3 cells (FIG. 9). Interestingly, as shown in in FIG. 9, the SKOV3 cells treated the anti-FOLR1 antibody and the SIRPα-Fc-CD40L chimeric protein, exhibited increased level of phagocytosis compared to the vehicle only-treated SKOV3 cells (p<0.0001), the SKOV3 cells treated with the anti-FOLR1 antibody alone, or the SKOV3 cells treated with the SIRPα-Fc-CD40L chimeric protein alone.

These results demonstrate that combination of SIRPα-Fc-CD40L with an ADCC/ADCP competent FOLR1 antibody enhances the phagocytosis of ovarian tumor cells. Therefore, these results indicate that a combination therapy of cancer with the SIRPα-Fc-CD40L chimeric protein and an anti-FOLR1 antibody is likely to produce a superior efficacy compared to both the SIRPα-Fc-CD40L chimeric protein and the anti-FOLR1 antibody. These data also suggest that a combination of the SIRPα-Fc-CD40L chimeric protein with ADCC/ADCP competent antibodies against tumor specific antigen targets (without limitation, e.g., PD-L1, CD47, CD38, FOLR1, CD123, SLAMF7 and BCMA) is likely to produce a superior efficacy compared to both the SIRPα-Fc-CD40L chimeric protein and the antibodies themselves.

### Example 9: In Vivo Anti-Tumor Activity of the SIRPα(CD172a)-Fc-CD40L Chimeric Protein in Combination with Paclitaxel

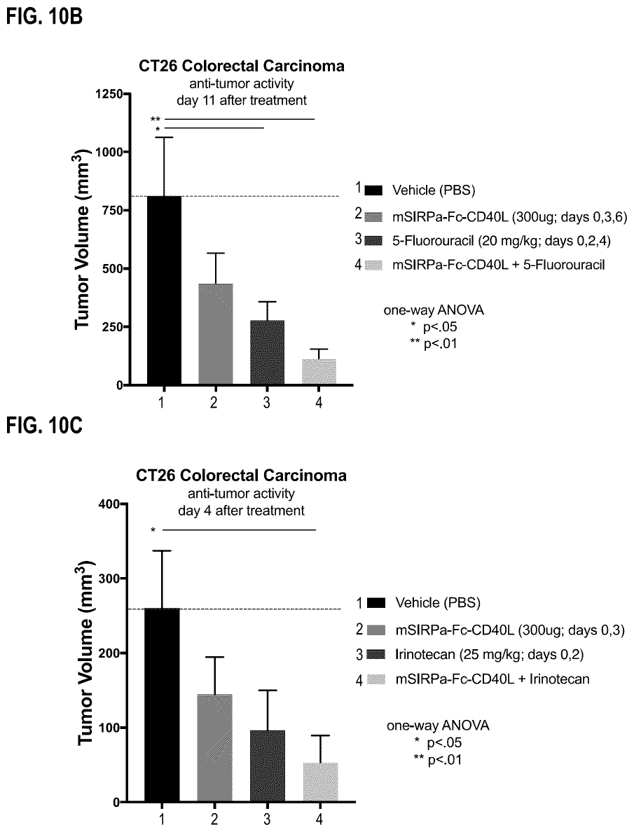

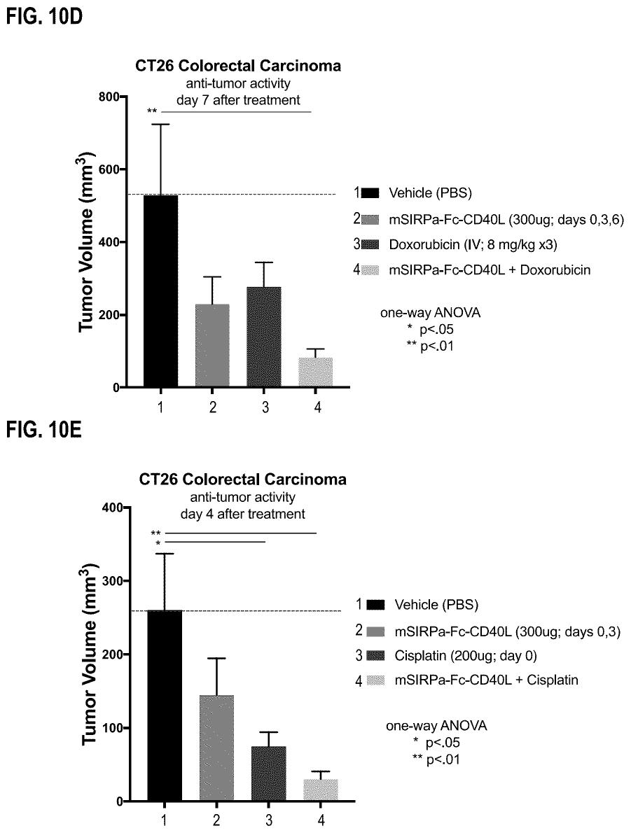

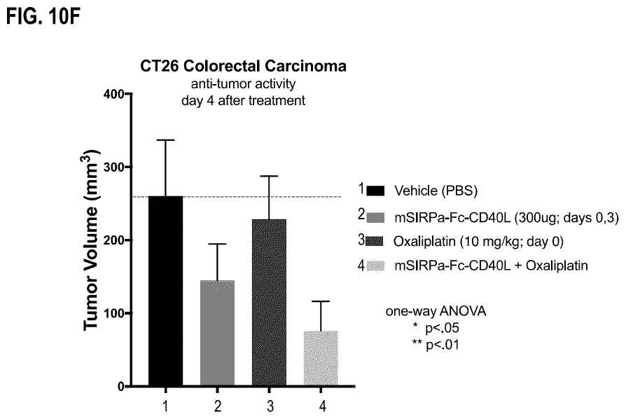

The efficacy of the SIRPα-Fc-CD40L chimeric protein was evaluated in combination with a spindle toxin. Towards that, the ability of paclitaxel and chimeric proteins to target and reduce tumor volume in vivo was determined. Briefly, BALB/C mice were inoculated with 500,000 CT26 tumor cells. When tumor volumes were approximately 50 to 60 mm³ (day 0), the mice were randomly distributed in the following treatment groups: (1) vehicle only control, (2) 300 μg of the SIRPα-Fc-CD40L chimeric protein alone, (3) 24 mg/kg paclitaxel alone, and (4) a combination 300 μg of the SIRPα-Fc-CD40L chimeric protein and 24 mg/kg paclitaxel. The mice were dosed on days 0, 3 and 6 via intraperitoneal injections. Tumors were measured with electronic calipers on day 14 and plotted using the GraphPad Prism software. As shown in FIG. 10A, the treatments with the SIRPα-Fc-CD40L chimeric protein alone and paclitaxel alone caused a reduction in tumor size. Interestingly, the combination treatment with the SIRPα-Fc-CD40L chimeric protein and paclitaxel caused a further reduction in tumor size (FIG. 10A).

These results demonstrate that the combination of the SIRPα-Fc-CD40L chimeric protein and a tubulin dynamics inhibitor (without limitations, e.g. paclitaxel, epothilone, docetaxel, discodermolide, vinblastine, vincristine, vinorelbine, vinflunine, dolastatins, halichondrins, hemiasterlins, and cryptophysin 52) may be beneficial than either single treatment, and thus, may be useful in the methods disclosed herein.

### Example 10: In Vivo Anti-Tumor Activity of the SIRPα(CD172a)-Fc-CD40L Chimeric Protein in Combination with 5-Fluorouracil

The efficacy of the SIRPα-Fc-CD40L chimeric protein was evaluated in combination with an antimetabolite or. Towards that, the ability of 5-fluorouracil and chimeric proteins to target and reduce tumor volume in vivo was determined. Briefly, BALB/C mice were inoculated with 500,000 CT26 tumor cells. When tumor volumes were approximately 50 to 60 mm³ (day 0), the mice were randomly distributed in the following treatment groups: (1) vehicle only control, (2) 300 μg of the SIRPα-Fc-CD40L chimeric protein alone, (3) 20 mg/kg 5-fluorouracil alone, and (4) a combination 300 μg of the SIRPα-Fc-CD40L chimeric protein and 20 mg/kg 5-fluorouracil. The mice were dosed via intraperitoneal injections as follows: the SIRPα-Fc-CD40L chimeric protein was administered on days 0, 3 and 6; and 5-fluorouracil was administered on days 0, 2 and 4. Tumors were measured with electronic calipers on day 11 and plotted using the GraphPad Prism software. As shown in FIG. 10B, the treatments with the SIRPα-Fc-CD40L chimeric protein alone and 5-fluorouracil alone caused a reduction in tumor size. Interestingly, the combination treatment with the SIRPα-Fc-CD40L chimeric protein and 5-fluorouracil caused a further reduction in tumor size compared to vehicle only control (p<0.01), the SIRPα-Fc-CD40L chimeric protein alone, and 20 mg/kg 5-fluorouracil alone (p<0.05) (FIG. 10B).

These results demonstrate that the combination of the SIRPα-Fc-CD40L chimeric protein and an antimetabolite and/or a thymidylate synthase inhibitor (without limitations, e.g. 5-fluorouracil (5-FU), 6-mercaptopurine (6-MP), capecitabine, cytarabine, floxuridine, fludarabine, gemcitabine, hydroxycarbamide, methotrexate, pemetrexed, phototrexate, raltitrexed, nolatrexed, ZD9331, and GS7904L) may be beneficial than either single treatment, and thus, may be useful in the methods disclosed herein.

### Example 11: In Vivo Anti-Tumor Activity of the SIRPα(CD172a)-Fc-CD40L Chimeric Protein in Combination with Irinotecan

The efficacy of the SIRPα-Fc-CD40L chimeric protein was evaluated in combination with a topoisomerase I inhibitor. Towards that, the ability of irinotecan and chimeric proteins to target and reduce tumor volume in vivo was determined. Briefly, BALB/C mice were inoculated with 500,000 CT26 tumor cells. When tumor volumes were approximately 50 to 60 mm³ (day 0), the mice were randomly distributed in the following treatment groups: (1) vehicle only control, (2) 300 μg of the SIRPα-Fc-CD40L chimeric protein alone, (3) 25 mg/kg irinotecan alone, and (4) a combination 300 μg of the SIRPα-Fc-CD40L chimeric protein and 25 mg/kg irinotecan. The mice were dosed on days 0 and 2 via intraperitoneal injections. Tumors were measured with electronic calipers on day 4 and plotted using the GraphPad Prism software. As shown in FIG. 10C, the treatments with the SIRPα-Fc-CD40L chimeric protein alone and irinotecan alone caused a reduction in tumor size. Interestingly, the combination treatment with the SIRPα-Fc-CD40L chimeric protein and irinotecan caused a further reduction in tumor size (FIG. 10C).

These results demonstrate that the combination of the SIRPα-Fc-CD40L chimeric protein and a topoisomerase I inhibitor (without limitations, e.g. camptothecin, belotecan topotecan, and irinotecan) may be beneficial than either single treatment, and thus, may be useful in the methods disclosed herein.

### Example 12: In Vivo Anti-Tumor Activity of the SIRPα(CD172a)-Fc-CD40L Chimeric Protein in Combination with Doxorubicin

The efficacy of the SIRPα-Fc-CD40L chimeric protein was evaluated in combination with an anthracycline. Towards that, the ability of doxorubicin and chimeric proteins to target and reduce tumor volume in vivo was determined. Briefly, BALB/C mice were inoculated with 500,000 CT26 tumor cells. When tumor volumes were approximately 50 to 60 mm³ (day 0), the mice were randomly distributed in the following treatment groups: (1) vehicle only control, (2) 300 μg of the SIRPα-Fc-CD40L chimeric protein alone, (3) 8 mg/kg doxorubicin alone, and (4) a combination 300 μg of the SIRPα-Fc-CD40L chimeric protein and 8 mg/kg doxorubicin. The mice were dosed with the SIRPα-Fc-CD40L chimeric protein on days 0, 3 and 6 via intraperitoneal injections, and with doxorubicin thrice through the tail vein. Tumors were measured with electronic calipers on day 7 and plotted using the GraphPad Prism software. As shown in FIG. 10D, the treatments with the SIRPα-Fc-CD40L chimeric protein alone and doxorubicin alone caused a reduction in tumor size. Interestingly, the combination treatment with the SIRPα-Fc-CD40L chimeric protein and doxorubicin caused a further reduction in tumor size (FIG. 10D).

These results demonstrate that the combination of the SIRPα-Fc-CD40L chimeric protein and an anthracycline (without limitations, e.g. daunorubicin, doxorubicin, epirubicin, idarubicin, mitoxantrone, and valrubicin) or a topoisomerase II inhibitor (without limitations, e.g. doxorubicin, epirubicin, valrubicin, daunorubicin, idarubicin, pitoxantrone, pixantrone, etoposide, teniposide, and amsacrine) may be beneficial than either single treatment, and thus, may be useful in the methods disclosed herein.

### Example 13: In Vivo Anti-Tumor Activity of the SIRPα(CD172a)-Fc-CD40L Chimeric Protein in Combination with a Platinum Drug

The efficacy of the SIRPα-Fc-CD40L chimeric protein was evaluated in combination with a platinum compound. Towards that, the ability of cisplatin and chimeric proteins to target and reduce tumor volume in vivo was determined. Briefly, BALB/C mice were inoculated with 500,000 CT26 tumor cells. When tumor volumes were approximately 50 to 60 mm³ (day 0), the mice were randomly distributed in the following treatment groups: (1) vehicle only control, (2) 300 μg of the SIRPα-Fc-CD40L chimeric protein alone, (3) 200 μg cisplatin alone, and (4) a combination 300 μg of the SIRPα-Fc-CD40L chimeric protein and 200 μg cisplatin. The mice were dosed via intraperitoneal injections with the SIRPα-Fc-CD40L chimeric protein on days 0 and 3, and with cisplatin on day 0. Tumors were measured with electronic calipers on day 4 and plotted using the GraphPad Prism software. As shown in FIG. 10E, the treatments with the SIRPα-Fc-CD40L chimeric protein alone and cisplatin alone caused a reduction in tumor size. Interestingly, the combination treatment with the SIRPα-Fc-CD40L chimeric protein and cisplatin caused a further reduction in tumor size (FIG. 10E).

In another experiment, the ability of oxaliplatin and chimeric proteins to target and reduce tumor volume in vivo was determined. Briefly, BALB/C mice were inoculated with 500,000 CT26 tumor cells. When tumor volumes were approximately 50 to 60 mm³ (day 0), the mice were randomly distributed in the following treatment groups: (1) vehicle only control, (2) 300 μg of the SIRPα-Fc-CD40L chimeric protein alone, (3) 10 mg/kg oxaliplatin alone, and (4) a combination 300 μg of the SIRPα-Fc-CD40L chimeric protein and 10 mg/kg oxaliplatin. The mice were dosed via intraperitoneal injections with the SIRPα-Fc-CD40L chimeric protein on days 0 and 3, and with oxaliplatin on day 0. Tumors were measured with electronic calipers on day 4 and plotted using the GraphPad Prism software. As shown in FIG. 10F, the treatments with the SIRPα-Fc-CD40L chimeric protein alone and oxaliplatin alone caused a reduction in tumor size. Interestingly, the combination treatment with the SIRPα-Fc-CD40L chimeric protein and oxaliplatin caused a further reduction in tumor size (FIG. 10F).

These results demonstrate that the combination of the SIRPα-Fc-CD40L chimeric protein and a platinum drug (without limitations, e.g. cisplatin, carboplatin and oxaliplatin) may be beneficial than either single treatment, and thus, may be useful in the methods disclosed herein.

### Example 14: In Vivo Anti-Tumor Activity of the SIRPα(CD172a)-Fc-CD40L Chimeric Protein in Combination with an Immune Checkpoint Inhibitor

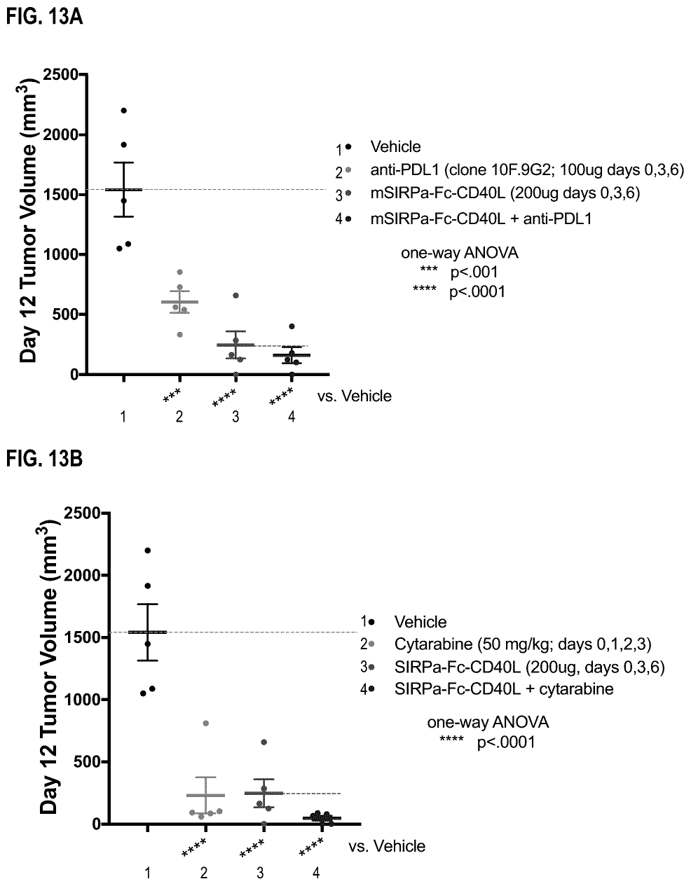

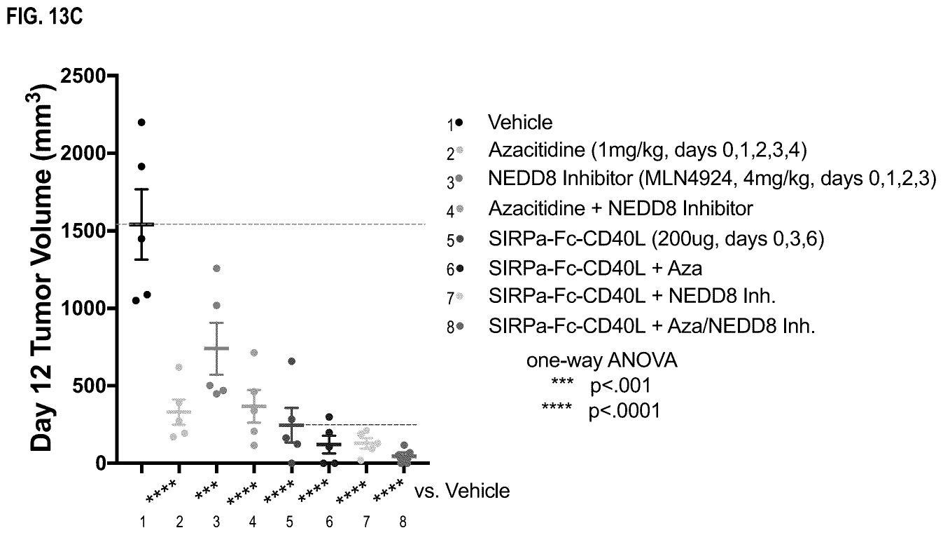

The efficacy of the SIRPα-Fc-CD40L chimeric protein was evaluated in combination with an immune checkpoint inhibitor. Towards that, the ability of an anti-PD-L1 antibody and chimeric proteins to target and reduce tumor volume in vivo was determined. Briefly, BALB/C mice were inoculated with 1×10⁶ A20 lymphoma cells. When tumor volumes were approximately 75 to 80 mm³ (day 0), the mice were randomly distributed in the following treatment groups: (1) vehicle only control, (2) 200 μg of the mouse SIRPα-Fc-CD40L chimeric protein alone, (3) 100 μg an anti-PD-L1 antibody (clone 10F.9G2), and (4) a combination 200 μg of the SIRPα-Fc-CD40L chimeric protein and 100 μg the anti-PD-L1 antibody. The mice were dosed via intraperitoneal injections with the SIRPα-Fc-CD40L chimeric protein on days 0, 3 and 6. Tumors were measured with electronic calipers on day 12 and plotted using the GraphPad Prism software. Dotted lines were drawn at the mean of the vehicle control group and at the mean of the mSIRPα-Fc-CD40L group. As shown in FIG. 13A, the treatments with the SIRPα-Fc-CD40L chimeric protein alone and the anti-PD-L1 antibody alone caused a reduction in tumor size. Interestingly, the combination treatment with the SIRPα-Fc-CD40L chimeric protein and the anti-PD-L1 antibody caused a further reduction in tumor size (FIG. 13A).

These results demonstrate that the combination of the SIRPα-Fc-CD40L chimeric protein and an immune checkpoint inhibitor (without limitations, e.g. anti-PD-1, anti-PD-L1 and anti-CTLA antibodies) may be beneficial than either single treatment, and thus, may be useful in the methods disclosed herein.

### Example 15: In Vivo Anti-Tumor Activity of the SIRPα(CD172a)-Fc-CD40L Chimeric Protein in Combination with an Antimetabolite/DNA Synthesis Inhibitor

The efficacy of the SIRPα-Fc-CD40L chimeric protein was evaluated in combination with an antimetabolite/DNA synthesis inhibitor. Towards that, the ability of cytarabine and the chimeric protein to target and reduce tumor volume in vivo was determined. Briefly, BALB/C mice were inoculated with 1×10⁶ A20 lymphoma cells. When tumor volumes were approximately 75 to 80 mm³ (day 0), the mice were randomly distributed in the following treatment groups: (1) vehicle only control, (2) 200 μg of the mouse SIRPα-Fc-CD40L chimeric protein alone, (3) 50 mg/kg cytarabine, and (4) a combination 200 μg of the SIRPα-Fc-CD40L chimeric protein and 50 mg/kg cytarabine. The mice were dosed via intraperitoneal injections with the SIRPα-Fc-CD40L chimeric protein on days 0, 3 and 6 and with cytarabine on days 0, 1, 2 and 3. Tumors were measured with electronic calipers on day 12 and plotted using the GraphPad Prism software. Dotted lines were drawn at the mean of the vehicle control group and at the mean of the mSIRPα-Fc-CD40L group. As shown in FIG. 13B, the treatments with the SIRPα-Fc-CD40L chimeric protein alone and the anti-PD-L1 antibody alone caused a reduction in tumor size. Interestingly, the combination treatment with the SIRPα-Fc-CD40L chimeric protein and the anti-PD-L1 antibody caused a further reduction in tumor size (FIG. 13B).

These results demonstrate that the combination of the SIRPα-Fc-CD40L chimeric protein and an antimetabolite/DNA synthesis inhibitor (without limitations, e.g. 5-fluorouracil (5-FU), capecitabine, floxuridine, cytarabine (ARA-C), gemcitabine, decitabine, and vidaza) may be beneficial than either single treatment, and thus, may be useful in the methods disclosed herein.

### Example 16: In Vivo Anti-Tumor Activity of the SIRPα(CD172a)-Fc-CD40L Chimeric Protein in Combination with a Hypomethylating Agent/an Epigenetic Regulator and/or a Protein Neddylation Inhibitor

The efficacy of the SIRPα-Fc-CD40L chimeric protein was evaluated in combination with a hypomethylating agent/an epigenetic regulator and/or a protein neddylation inhibitor. Towards that, the ability of an anti-PD-L1 antibody and chimeric proteins to target and reduce tumor volume in vivo was determined. Briefly, BALB/C mice were inoculated with 1×10⁶ A20 lymphoma cells. When tumor volumes were approximately 75 to 80 mm³ (day 0), the mice were randomly distributed in the following treatment groups: (1) vehicle only control, (2) 1 mg/kg azacitidine alone, (3) 4 mg/kg pevonedistat (MLN4924) alone, (4) a combination 1 mg/kg azacitidine and 4 mg/kg pevonedistat (MLN4924), (5) 200 μg of the SIRPα-Fc-CD40L chimeric protein alone, (6) a combination 1 mg/kg azacitidine and 200 μg of the SIRPα-Fc-CD40L chimeric protein, (7) a combination of 4 mg/kg pevonedistat (MLN4924) and 200 μg of the SIRPα-Fc-CD40L chimeric protein, and (8) a combination of 1 mg/kg azacitidine, 4 mg/kg pevonedistat (MLN4924) and 200 μg of the SIRPα-Fc-CD40L chimeric protein. The mice were dosed via intraperitoneal injections with the SIRPα-Fc-CD40L chimeric protein on days 0, 3 and 6, with pevonedistat (MLN4924) on days 0, 1, 2 and 3, and with azacitidine on days 0, 1, 2, 3 and 4. Tumors were measured with electronic calipers on day 12 and plotted using the GraphPad Prism software. Dotted lines were drawn at the mean of the vehicle control group and at the mean of the mSIRPα-Fc-CD40L group.

As shown in FIG. 13C, the treatments with azacitidine alone, pevonedistat (MLN4924) alone, and the SIRPα-Fc-CD40L chimeric protein alone caused a reduction in tumor size. Interestingly, as shown in FIG. 13C, the extent of tumor reduction caused by the combinations of the SIRPα-Fc-CD40L chimeric protein with either azacitidine and pevonedistat (MLN4924) was greater than that caused by each of the SIRPα-Fc-CD40L chimeric protein alone, azacitidine alone, and pevonedistat (MLN4924) alone. In contrast, the extent of tumor reduction caused by the combination of azacitidine and pevonedistat (MLN4924) was not greater than that caused by azacitidine alone or pevonedistat (MLN4924) alone (FIG. 13C). Interestingly, the combination treatment with the SIRPα-Fc-CD40L chimeric protein, azacitidine and pevonedistat (MLN4924) caused a further reduction in tumor size (FIG. 13B), which was greater than that caused by all other treatments. Thus, while the extent of tumor reduction caused by the combination of azacitidine and pevonedistat (MLN4924) was not greater than that caused by azacitidine alone or pevonedistat (MLN4924) alone, the extent of tumor reduction caused by the combination of the SIRPα-Fc-CD40L chimeric protein, azacitidine and pevonedistat (MLN4924) was greater than that caused by the combination of the SIRPα-Fc-CD40L chimeric protein and azacitidine, or the combination of the SIRPα-Fc-CD40L chimeric protein and pevonedistat (MLN4924) alone.

As illustrated by the combination of azacitidine and pevonedistat (MLN4924), combinations of anticancer medicines do not always produce a beneficial effect. However, these results demonstrate that the combination of the SIRPα-Fc-CD40L chimeric protein and a hypomethylating agent/an epigenetic regulator (without limitations, e.g., azacitidine, 5-aza-2′-deoxycytidine, suberoylanilide hydroxamic acid (saha), romidepsin, belinostat, panobinostat, and chidamide) may be beneficial than either single treatment, and thus, may be useful in the methods disclosed herein. These results also demonstrate that the combination of the SIRPα-Fc-CD40L chimeric protein and a protein neddylation inhibitor (without limitations, e.g. pevonedistat (MLN4924)) may be effective against cancer may be beneficial than either single treatment, and thus, may be useful in the methods disclosed herein. Further, these results also demonstrate that the combination of the SIRPα-Fc-CD40L chimeric protein, the combination of the SIRPα-Fc-CD40L chimeric protein and a hypomethylating agent/an epigenetic regulator (without limitations, e.g. azacitidine, 5-aza-2′-deoxycytidine, suberoylanilide hydroxamic acid (saha), romidepsin, belinostat, panobinostat, and chidamide), and a protein neddylation inhibitor (without limitations, e.g. pevonedistat (MLN4924) may be effective against cancer may be beneficial than either single treatment or double treatments, and thus, may be useful in the methods disclosed herein.

### Example 17: The Amplification of the Phagocytosis-Stimulating Activity of the SIRPα-Fc-CD40L Chimeric Protein by Chemotherapeutic Agents Correlates with the Induction of CD47 and/or Pro-Phagocytic Signals

Molecular basis for the observed potentiation of phagocytosis-stimulating activity of the SIRPα-Fc-CD40L chimeric protein by chemotherapeutic agents, the surface expression of pro- and anti-phagocytic signals was studied.

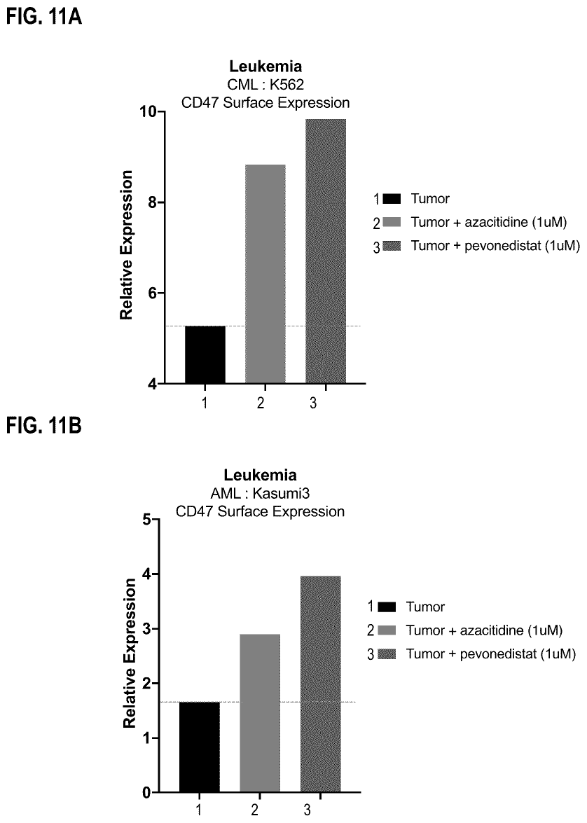

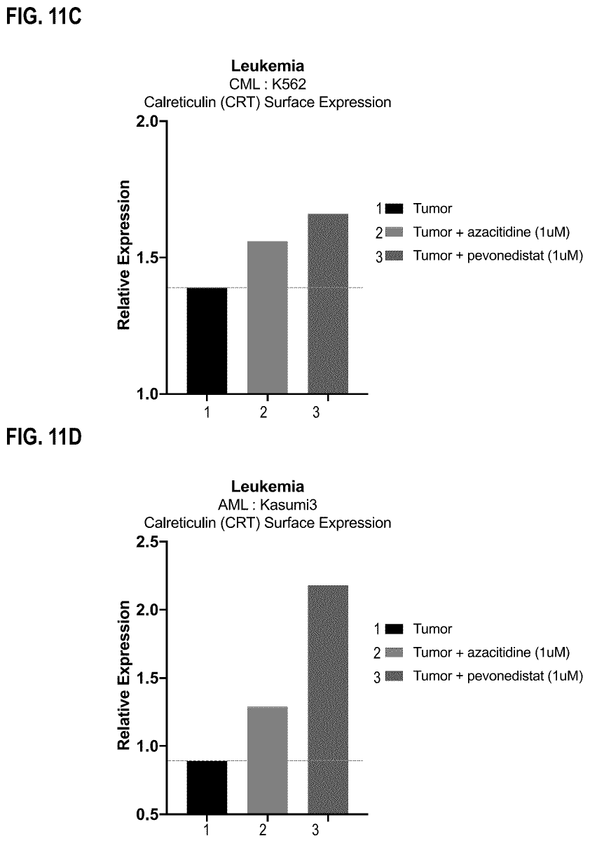

Briefly, the K652 human chronic myelogenous leukemia cells were incubated overnight in the presence of vehicle only control, 1 μM azacitidine or 1 μM pevonedistat. The following day, the K652 cells were analyzed by flow cytometry for surface expression of CD47 or calreticulin (CRT). As shown in FIG. 11A, both azacitidine and pevonedistat (MLN4924) induced the expression of CD47 in K652 cells compared to the vehicle only-treated K652 cells. These results demonstrate that the observed potentiation of phagocytosis-stimulating activity of the SIRPα-Fc-CD40L chimeric protein by azacitidine correlates with the induction of CD47, a target of the SIRPα-Fc-CD40L chimeric protein.

Interestingly, as shown in FIG. 11C, both azacitidine and pevonedistat (MLN4924) induced the expression of calreticulin, a pro-phagocytic marker, in K652 cells compared to the vehicle only-treated K652 cells. These results demonstrate that the observed potentiation of phagocytosis-stimulating activity of the SIRPα-Fc-CD40L chimeric protein by azacitidine correlates with the induction of calreticulin, a pro-apoptotic protein.

In another experiment, the Kasumi-3 human acute myelocytic leukemia (AML) cells were incubated overnight in the presence of vehicle only control, 1 μM pevonedistat (MLN4924) or 1 μM pevonedistat. The following day, the Kasumi-3 cells were analyzed by flow cytometry for surface expression of CD47 or calreticulin (CRT). As shown in FIG. 11B, both pevonedistat (MLN4924) and pevonedistat (MLN4924) induced the expression of CD47 in Kasumi-3 cells compared to the vehicle only-treated Kasumi-3 cells. These results demonstrate that the observed potentiation of phagocytosis-stimulating activity of the SIRPα-Fc-CD40L chimeric protein by pevonedistat (MLN4924) correlates with the induction of CD47, a target of the SIRPα-Fc-CD40L chimeric protein.

Further, as shown in FIG. 11D, both pevonedistat (MLN4924) and pevonedistat (MLN4924) induced the expression of calreticulin, a pro-phagocytic marker, in Kasumi-3 cells compared to the vehicle only-treated Kasumi-3 cells. These results demonstrate that the observed potentiation of phagocytosis-stimulating activity of the SIRPα-Fc-CD40L chimeric protein by pevonedistat (MLN4924) correlates with the induction of calreticulin, a pro-apoptotic protein.

Collectively, these results demonstrate that the observed potentiation of phagocytosis-stimulating activity of the SIRPα-Fc-CD40L chimeric protein by chemotherapeutic agents correlates with the induction of CD47, and/or pro-phagocytic signals. Accordingly, these results indicate that the induction of CD47 and/or pro-phagocytic signals may be used in the methods of predicting response or methods of selecting patients for therapy disclosed herein.

### Example 18: APR-246, the Reactivator of Mutated p53 Induces p53 and Pro-Phagocytic Signals

To understand molecular basis for the observed potentiation of phagocytosis-stimulating activity of the SIRPα-Fc-CD40L chimeric protein by APR-246, the surface expression of p53 and anti-phagocytic signals was studied.

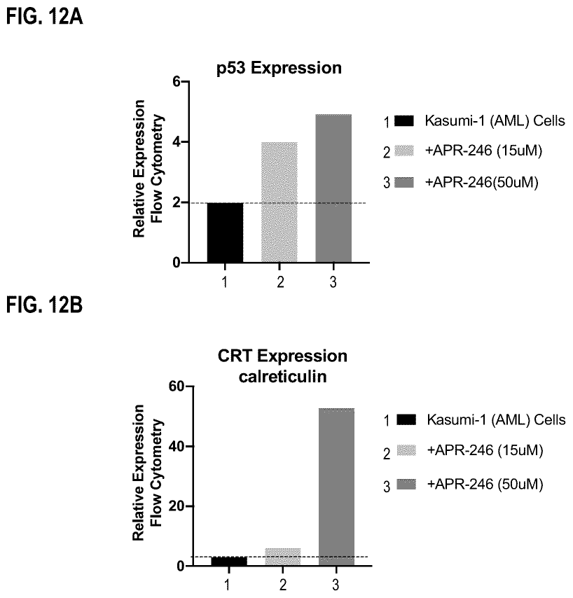

Briefly, the Kasumi-1 human acute myelocytic leukemia (AML) cells were incubated overnight in the presence of vehicle only control, 15 μM APR-246, or 50 μM APR-246. The following day, the Kasumi-1 cells were analyzed by flow cytometry for surface expression of p53 and calreticulin (CRT). As shown in FIG. 12A, APR-246 caused a dose-dependent induction the expression of p53 in Kasumi-1 cells compared to the vehicle only-treated Kasumi-1 cells.

Interestingly, as shown in FIG. 12B, APR-246 induced the expression of calreticulin, a pro-phagocytic marker, in Kasumi-1 cells compared to the vehicle only-treated Kasumi-1 cells. These results demonstrate that the observed potentiation of phagocytosis-stimulating activity of the SIRPα-Fc-CD40L chimeric protein by azacitidine correlates with the induction of calreticulin, a pro-apoptotic protein.

These results taken together with the preceding example demonstrate that chemotherapeutic agents induce pro-phagocytic signals. Accordingly, these results indicate that the induction pro-phagocytic signals may be used in the methods of predicting response or methods of selecting patients for therapy disclosed herein.

### Example 19: Azacitidine and Venetoclax Increased the Expression of the Pro-Apoptotic Markers and Amplify the Phagocytosis-Stimulating Activity of the SIRPα-Fc-CD40L Chimeric Protein

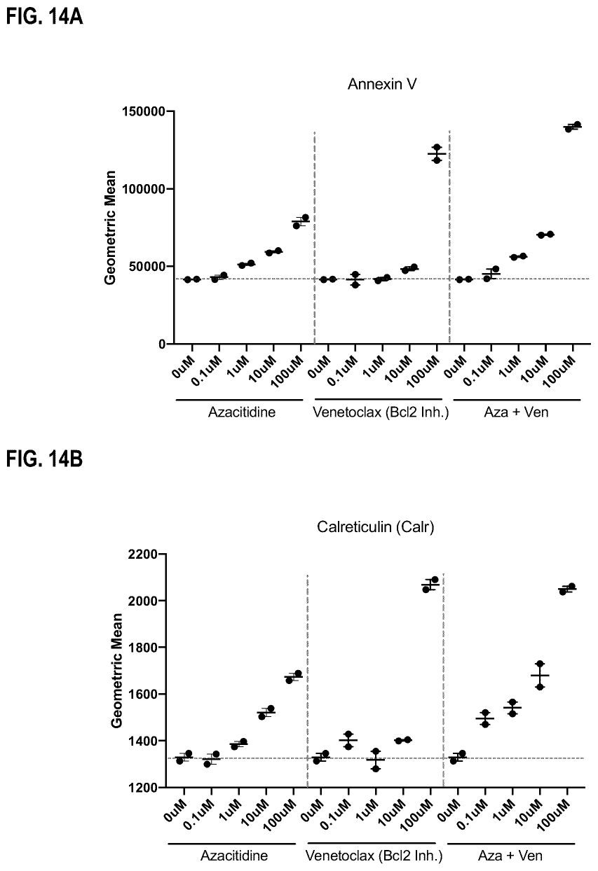

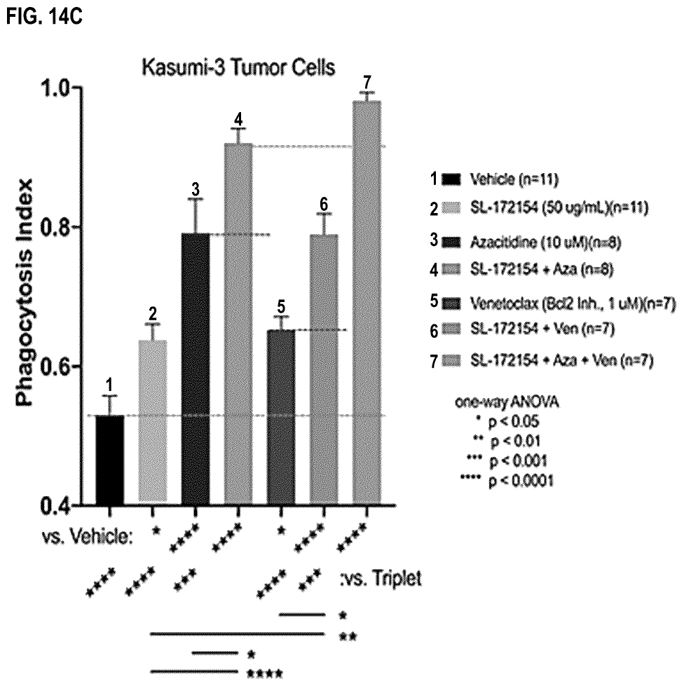

The surface expression of apoptosis marker annexin 3 or the pro-apoptotic protein calreticulin (CRT) was studied. Briefly, the Kasumi-3 cells were incubated overnight in the presence of vehicle only control, increasing amounts of azacitidine or venetoclax, or a combination of azacitidine and venetoclax. The following day, the cells were analyzed by flow cytometry for surface expression of annexin or calreticulin (CRT). As shown in FIG. 14A, both azacitidine and venetoclax induced the expression of annexin V in Kasumi-3 cells in a dose-dependent manner compared to the vehicle only-treated Kasumi-3 cells. In addition, except for very high concentrations of azacitidine and venetoclax (100 μM each), the apoptosis induced by azacitidine and venetoclax was not additive compared to corresponding single treatments (FIG. 14A). As shown in FIG. 14B, both azacitidine and venetoclax induced the expression of calreticulin in Kasumi-3 cells in a dose-dependent manner compared to the vehicle only-treated Kasumi-3 cells. In addition, except for very high concentrations of azacitidine and venetoclax (100 μM each), the apoptosis induced by azacitidine and venetoclax was not additive compared to corresponding single treatments (FIG. 14B).

These results demonstrate, inter alia, that both azacitidine and venetoclax induced apoptosis and proapoptotic protein calreticulin in Kasumi-3 cells.

The effect of the treatment of cancer cells by an azacitidine and/or venetoclax on the phagocytosis-stimulating activity of the SIRPα-Fc-CD40L chimeric protein was determined.

Briefly, the Kasumi-3 AML cells were labeled with a green fluorescent tracker and co-cultured with human macrophages and treated with (1) vehicle alone control, (2) 50 μg/ml of the SIRPα-Fc-CD40L chimeric protein, (3) 10 μM azacitidine, (4) 50 μg/ml of the SIRPα-Fc-CD40L chimeric protein+10 μM azacitidine, (5) 1 μM venetoclax, (6) 50 μg/ml of the SIRPα-Fc-CD40L chimeric protein+1 μM venetoclax, or (7) 50 μg/ml of the SIRPα-Fc-CD40L chimeric protein+1 μM venetoclax+10 μM azacitidine. The cells were incubated at 37° C. in the presence of 5% CO₂ for 4 hours. After this incubation, the cells were harvested and treated with an anti-CD11b antibody (a macrophage marker) and were analyzed using flow cytometry. Positive phagocytosis was determined by the overlap in signals of tumor (the green fluorescent tracker) and macrophage (anti-CD11b antibody staining). A phagocytosis index was calculated by setting the maximum phagocytosis value to 1, and then normalizing all other replicates accordingly. The phagocytosis index was plotted for the indicated treatments. As shown in FIG. 14C, the Kasumi-3 cells that were treated with vehicle alone control were phagocytized by the human macrophages at a background level. Compared to the vehicle only-treated Kasumi-3 cells, the Kasumi-3 cells treated with the SIRPα-Fc-CD40L chimeric protein alone (p<0.05), the azacitidine alone (p<0.0001), or venetoclax alone (p<0.05) exhibited an increased level of phagocytosis (FIG. 14C). As shown in in FIG. 14C, the cells treated a combination of azacitidine and the SIRPα-Fc-CD40L chimeric protein exhibited increased level of phagocytosis compared to the vehicle only-treated cells (p<0.0001), the cells treated with azacitidine alone (p<0.05), or the cells treated with the SIRPα-Fc-CD40L chimeric protein alone (p<0.0001). Further, as shown in in FIG. 14C, the cells treated a combination of venetoclax and the SIRPα-Fc-CD40L chimeric protein exhibited increased level of phagocytosis compared to the vehicle only-treated cells (p<0.0001), the cells treated with venetoclax alone (p<0.05), or the cells treated with the SIRPα-Fc-CD40L chimeric protein alone (p<0.001). Interestingly, as shown in in FIG. 14C, the cells treated a combination of azacitidine, venetoclax and the SIRPα-Fc-CD40L chimeric protein exhibited increased level of phagocytosis compared to the vehicle only-treated cells (p<0.0001), the cells treated with azacitidine alone (p<0.001), the cells treated with venetoclax alone (p<0.0001), or the cells treated with the SIRPα-Fc-CD40L chimeric protein alone (p<0.0001), the cells treated the combination of azacitidine and the SIRPα-Fc-CD40L chimeric protein, or the cells treated the combination of venetoclax and the SIRPα-Fc-CD40L chimeric protein (p<0.001).

These results demonstrate that combination of SIRPα-Fc-CD40L with azacitidine and/or venetoclax the phagocytosis of tumor cells. Therefore, these results indicate, inter alia, that (1) a combination therapy of cancer with the SIRPα-Fc-CD40L chimeric protein and azacitidine is likely to produce a superior efficacy compared to both the SIRPα-Fc-CD40L chimeric protein and azacitidine; (2) a combination therapy of cancer with the SIRPα-Fc-CD40L chimeric protein and venetoclax is likely to produce a superior efficacy compared to both the SIRPα-Fc-CD40L chimeric protein and venetoclax, and (3) a combination therapy of cancer with the SIRPα-Fc-CD40L chimeric protein, azacitidine and venetoclax is likely to produce a superior efficacy compared to each of the SIRPα-Fc-CD40L chimeric protein, azacitidine and venetoclax.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior disclosure.

As used herein, all headings are simply for organization and are not intended to limit the disclosure in any manner. The content of any individual section may be equally applicable to all sections.

### EQUIVALENTS

While the disclosure has been disclosed in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A method for treating a cancer in a subject in need thereof comprising:
administering to the subject a first pharmaceutical composition comprising a heterologous chimeric protein comprising:
(a) a first domain comprising a portion of the extracellular domain of SIRPα(CD172a), wherein the portion is capable of binding a SIRPα(CD172a) ligand,
(b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and
(c) a linker linking the first domain and the second domain; and
administering to the subject a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof.

2. The method of claim 1, wherein the first pharmaceutical composition and the second pharmaceutical composition are administered simultaneously.

3. The method of claim 1, wherein the first pharmaceutical composition is administered after the second pharmaceutical composition is administered.

4. The method of claim 1, wherein the first pharmaceutical composition is administered before the second pharmaceutical composition is administered.

5. The method of any one of claims 1 to 3, wherein the dose of the first pharmaceutical composition is less than the dose of the first pharmaceutical composition administered to a subject who has not undergone or is not undergoing treatment with the second pharmaceutical composition.

6. The method of any one of claim 1, 2, or 4, wherein the dose of the second pharmaceutical composition administered is less than the dose of the second pharmaceutical composition administered to a subject who has not undergone or is not undergoing treatment with the first pharmaceutical composition.

7. The method of any one of claims 1 to 6, wherein the subject has an increased chance of survival, without gastrointestinal inflammation and weight loss, and/or a reduction in tumor size or cancer prevalence when compared to a subject who has only undergone or is only undergoing treatment with the first pharmaceutical composition.

8. The method of any one of claims 1 to 7, wherein the subject has an increased chance of survival, without gastrointestinal inflammation and weight loss, and/or a reduction in tumor size or cancer prevalence when compared to a subject who has only undergone or is only undergoing treatment with the second pharmaceutical composition.

9. A method for treating a cancer in a subject comprising:
administering to the subject a pharmaceutical composition comprising a heterologous chimeric protein comprising:
(a) a first domain comprising a portion of the extracellular domain of SIRPα(CD172a), wherein the portion is capable of binding a SIRPα(CD172a) ligand,
(b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and
(c) a linker linking the first domain and the second domain;
wherein the subject has undergone or is undergoing treatment with a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof.

10. The method of claim 9, wherein the dose of the pharmaceutical composition administered to the subject is less than the dose of the pharmaceutical composition that is administered to a subject who has not undergone or is not undergoing treatment with the second pharmaceutical composition.

11. A method for treating a cancer in a subject comprising:
administering to the subject a second pharmaceutical composition comprising an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof;
wherein the subject has undergone or is undergoing treatment with a heterologous chimeric protein comprising:
(a) a first domain comprising a portion of the extracellular domain of SIRPα(CD172a), wherein the portion is capable of binding a SIRPα(CD172a) ligand,
(b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and
(c) a linker linking the first domain and the second domain.

12. The method of claim 11, wherein the dose of the pharmaceutical composition provided to the subject is less than the dose of the pharmaceutical composition that is provided to a subject who has not undergone or is not undergoing treatment with the heterologous chimeric protein.

13. The method of any one of the claims 1 to 12, wherein the second pharmaceutical composition comprises a hypomethylating agent/epigenetic regulator.

14. The method of claim 13, wherein the hypomethylating agent/epigenetic regulator is selected from azacitidine, 5-aza-2′-deoxycytidine, suberoylanilide hydroxamic acid (saha), romidepsin, belinostat, panobinostat, and chidamide.

15. The method of claim 14, wherein the hypomethylating agent/epigenetic regulator is azacitidine.

16. The method of any one of the claims 1 to 12, wherein the second pharmaceutical composition comprises a proteasomal inhibitor.

17. The method of claim 16, wherein the proteasomal inhibitor is selected from bortezomib, carfilzomib and ixazomib.

18. The method of claim 17, wherein the proteasomal inhibitor is bortezomib.

19. The method of any one of the claims 1 to 12, wherein the second pharmaceutical composition comprises an anti-metabolite.

20. The method of claim 19, wherein the antimetabolite is selected from 5-fluorouracil (5-FU), capecitabine, floxuridine, cytarabine (ARA-C), gemcitabine, decitabine, and vidaza.

21. The method of claim 20, wherein the antimetabolite is cytarabine (ARA-C).

22. The method of any one of the claims 1 to 12, wherein the second pharmaceutical composition comprises a DNA synthesis inhibitor.

23. The method of claim 22, wherein the DNA synthesis inhibitor is selected from 5-fluorouracil (5-FU), capecitabine, floxuridine, cytarabine (ARA-C), gemcitabine, decitabine, and vidaza.

24. The method of claim 23, wherein the DNA synthesis inhibitor is cytarabine (ARA-C) or 5-fluorouracil (5-FU).

25. The method of any one of the claims 1 to 12, wherein the second pharmaceutical composition comprises an immune checkpoint inhibitor.

26. The method of claim 25, wherein the immune checkpoint inhibitor comprises an agent that inhibits a pathway selected from CTLA-4, PD-1 and PD-L1.

27. The method of claim 26, wherein the immune checkpoint inhibitor comprises an anti-PD-L1 antibody.

28. The method of claim 27, wherein the anti-PD-L1 antibody is selected from atezolizumab, durvalumab, avelumab, envafolimab, BMS-936559, CK-301, CS-1001, SHR-1316 (HTI-1088), CBT-502 (TQB-2450) and BGB-A333.

29. The method of any one of the claims 1 to 12, wherein the second pharmaceutical composition comprises an anthracyline.

30. The method of claim 29, wherein the anthracycline is selected from daunorubicin, doxorubicin, epirubicin, idarubicin, mitoxantrone, and valrubicin.

31. The method of claim 30, wherein the anthracycline is doxorubicin.

32. The method of any one of the claims 1 to 12, wherein the second pharmaceutical composition comprises a topoisomerase II inhibitor.

33. The method of claim 32, wherein the topoisomerase II inhibitor is selected from doxorubicin, epirubicin, valrubicin, daunorubicin, idarubicin, pitoxantrone, pixantrone, etoposide, teniposide, and amsacrine.

34. The method of claim 33, wherein the topoisomerase II inhibitor is doxorubicin.

35. The method of any one of the claims 1 to 12, wherein the second pharmaceutical composition comprises a Bcl2 inhibitor.

36. The method of claim 35, wherein the Bcl2 inhibitor is selected from oblimersen, navitoclax (ABT-263), venetoclax (ABT-199), obatoclax mesylate (GX15-070), and AT-101.

37. The method of claim 36, wherein the Bcl2 inhibitor is venetoclax.

38. The method of any one of the claims 1 to 12, wherein the second pharmaceutical composition comprises a protein neddylation inhibitor.

39. The method of claim 38, wherein the protein neddylation inhibitor is pevonedistat (MLN4924).

40. The method of any one of the claims 1 to 12, wherein the second pharmaceutical composition comprises a microtubule-targeting agent.

41. The method of claim 40, wherein the microtubule-targeting agent is selected from paclitaxel, epothilone, docetaxel, discodermolide, vinblastine, vincristine, vinorelbine, vinflunine, dolastatins, halichondrins, hemiasterlins, and cryptophysin 52.

42. The method of claim 41, wherein the microtubule-targeting agent is paclitaxel.

43. The method of any one of the claims 1 to 12, wherein the second pharmaceutical composition comprises a thymidylate synthase (TS) inhibitor.

44. The method of claim 43, wherein the thymidylate synthase (TS) inhibitor is selected from 5-fluorouracil (5-FU), 6-mercaptopurine (6-MP), capecitabine, cytarabine, floxuridine, fludarabine, gemcitabine, hydroxycarbamide, methotrexate, pemetrexed, phototrexate, raltitrexed, nolatrexed, ZD9331, and GS7904L.

45. The method of claim 44, wherein the thymidylate synthase (TS) inhibitor is 5-fluorouracil (5-FU).

46. The method of any one of the claims 1 to 12, wherein the second pharmaceutical composition comprises a platinum drug.

47. The method of claim 46, wherein the platinum drug is selected from cisplatin, carboplatin, oxaliplatin, nedaplatin, heptaplatin and lobaplatin.

48. The method of claim 47, wherein the platinum drug is cisplatin.

49. The method of claim 47, wherein the platinum drug is oxaliplatin.

50. The method of any one of the claims 1 to 12, wherein the second pharmaceutical composition comprises a topoisomerase I inhibitor.

51. The method of claim 50, wherein the topoisomerase I inhibitor is selected from camptothecin, belotecan topotecan, and irinotecan.

52. The method of claim 50, wherein the topoisomerase I inhibitor is irinotecan.

53. The method of any one of the claims 1 to 12, wherein the second pharmaceutical composition comprises an anti-BCMA antibody.

54. The method of claim 53, wherein the anti-BCMA antibody is C12A3.2.

55. The method of any one of the claims 1 to 12, wherein the second pharmaceutical composition comprises an anti-CD38 antibody.

56. The method of claim 55, wherein the anti-CD38 antibody is selected from daratumumab and isatuximab.

57. The method of claim 56, wherein the anti-CD38 antibody is daratumumab.

58. The method of any one of the claims 1 to 12, wherein the second pharmaceutical composition comprises an immunomodulatory imide drug (IMiD).

59. The method of claim 58, wherein the immunomodulatory imide drug (IMiD) is selected from apremilast, thalidomide, lenalidomide, and pomalidomide.

60. The method of claim 59, wherein the immunomodulatory imide drug (IMiD) is lenalidomide or pomalidomide.

61. The method of any one of the claims 1 to 12, wherein the second pharmaceutical composition comprises an anti-SLAMF7 antibody.

62. The method of claim 61, wherein the anti-SLAMF7 antibody is elotuzumab.

63. The method of any one of the claims 1 to 12, wherein the second pharmaceutical composition comprises an anti-CD123 antibody.

64. The method of claim 63, wherein the anti-CD123 antibody is talacotuzumab.

65. The method of any one of the claims 1 to 12, wherein the second pharmaceutical composition comprises a reactivator of mutated p53.

66. The method of claim 65, wherein the reactivator of mutated p53 is Prima-1 or APR-246.

67. The method of claim 66, wherein the reactivator of mutated p53 is APR-246.

68. The method of any one of the claims 1 to 12, wherein the second pharmaceutical composition comprises an anti-FOLR1 antibody, optionally wherein the anti-FOLR1 antibody is selected from farletuzumab and mirvetuximab.

69. The method of claim 68, wherein the anti-FOLR1 antibody is farletuzumab.

70. The method of any one of the claims 1 to 69, wherein the heterologous chimeric protein comprises a first domain which comprises substantially the entire extracellular domain of SIRPα(CD172a) and/or a second domain which comprises substantially the entire extracellular domain of CD40L, OX40L, or LIGHT.

71. The method of any one of the claims 1 to 70, wherein the heterologous chimeric protein comprises:
(a) a first domain comprising a portion of SIRPα(CD172a),
(b) a second domain comprising a portion of CD40L, OX40L, or LIGHT, and
(c) a linker comprising a hinge-CH2-CH3 Fc domain.

72. The method of any one of the claims 1 to 71, wherein the linker is a polypeptide selected from a flexible amino acid sequence, an IgG hinge region, and an antibody sequence.

73. The method of any one of the claims 1 to 71, wherein the linker comprises at least one cysteine residue capable of forming a disulfide bond and/or comprises a hinge-CH2-CH3 Fc domain.

74. The method of claim 73, wherein the linker comprises a hinge-CH2-CH3 Fc domain derived from IgG1 or IgG4, e.g., human IgG4 or human IgG4.

75. The method of claim 74, wherein the linker comprises an amino acid sequence that is at least 95% identical to the amino acid sequence of SEQ ID NO: 1, SEQ ID NO: 2, or SEQ ID NO: 3.

76. The method of any one of the claims 1 to 75, wherein the first domain comprises an amino acid sequence that is at least 90%, or at least 93%, at least 95%, or at least 96%, or at least 98%, or at least 99% identical to the amino acid sequence of SEQ ID NO: 57.

77. The method of any one of the claims 1 to 76, wherein the second domain comprises an amino acid sequence that is at least 90%, or at least 93%, at least 95%, or at least 96%, or at least 98%, or at least 99% identical to the amino acid sequence of SEQ ID NO: 58, SEQ ID NO: 59 or SEQ ID NO: 62.

78. The method of claim 77, wherein the second domain comprises an amino acid sequence that is at least 90%, or at least 93%, at least 95%, or at least 96%, or at least 98%, or at least 99% identical to the amino acid sequence of SEQ ID NO: 58.

79. The method of any one of the claims 1 to 77, wherein the heterologous chimeric protein comprises an amino acid sequence that is at least 90%, or at least 93%, at least 95%, or at least 96%, or at least 98%, or at least 99% identical to the amino acid sequence of SEQ ID NO: 60, SEQ ID NO: 61, or SEQ ID NO: 63.

80. The method of claim 78 or claim 79, wherein the heterologous chimeric protein comprises an amino acid sequence that is at least 90%, or at least 93%, at least 95%, or at least 96%, or at least 98%, or at least 99% identical to the amino acid sequence of SEQ ID NO: 60.

81. The method of any one of claims 1 to 80, wherein the cancer is or is related to a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

82. A medicament for treating cancer, the medicament comprising a first pharmaceutical composition and a second pharmaceutical composition, wherein the first pharmaceutical composition and the second pharmaceutical composition are to be administered simultaneously or sequentially in combination,
wherein the first pharmaceutical composition comprises a heterologous chimeric protein comprising:
(a) a first domain comprising a portion of the extracellular domain of SIRPα(CD172a), wherein the portion is capable of binding a SIRPα(CD172a) ligand,
(b) a second domain comprising a portion of the extracellular domain of CD40L, wherein the portion is capable of binding a CD40L receptor, a portion of the extracellular domain of OX40L, wherein the portion is capable of binding an OX40L receptor, or a portion of the extracellular domain of LIGHT, wherein the portion is capable of binding a LIGHT receptor, and
(c) a linker linking the first domain and the second domain; and
wherein the second pharmaceutical composition comprises an anticancer agent selected from a hypomethylating agent/epigenetic regulator, a proteasomal inhibitor, an anti-metabolite, a DNA synthesis inhibitor, an immune checkpoint inhibitor, an anthracycline, a topoisomerase II inhibitor, an innate immune checkpoint inhibitor, a Bcl2 inhibitor, a protein neddylation inhibitor, a microtubule-targeting agent, a thymidylate synthase (TS) inhibitor, a platinum drug, a topoisomerase I inhibitor, an anti-BCMA antibody, an anti-CD38 antibody, an immunomodulatory imide drug (IMiD), an anti-SLAMF7 antibody, an anti-CD123 antibody, a reactivator of mutated p53, and anti-FOLR1 antibody, or a combination thereof.


SEQUENCE LISTING (63 sequences)

SEQ ID NO: 1 - protein, 217 aa, Artificial Sequence
  Synthetic Polypeptide
APEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

SEQ ID NO: 2 - protein, 217 aa, Artificial Sequence
  Synthetic Polypeptide
APEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGK

SEQ ID NO: 3 - protein, 217 aa, Artificial Sequence
  Synthetic Polypeptide
APEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 4 - protein, 11 aa, Artificial Sequence
  Synthetic Polypeptide
SKYGPPCPSCP

SEQ ID NO: 5 - protein, 11 aa, Artificial Sequence
  Synthetic Polypeptide
SKYGPPCPPCP

SEQ ID NO: 6 - protein, 6 aa, Artificial Sequence
  Synthetic Polypeptide
SKYGPP

SEQ ID NO: 7 - protein, 6 aa, Artificial Sequence
  Synthetic Polypeptide
IEGRMD

SEQ ID NO: 8 - protein, 9 aa, Artificial Sequence
  Synthetic Polypeptide
GGGVPRDCG

SEQ ID NO: 9 - protein, 15 aa, Artificial Sequence
  Synthetic Polypeptide
IEGRMDGGGGAGGGG

SEQ ID NO: 10 - protein, 8 aa, Artificial Sequence
  Synthetic Polypeptide
GGGSGGGS

SEQ ID NO: 11 - protein, 12 aa, Artificial Sequence
  Synthetic Polypeptide
GGGSGGGGSGGG

SEQ ID NO: 12 - protein, 14 aa, Artificial Sequence
  Synthetic Polypeptide
EGKSSGSGSESKST

SEQ ID NO: 13 - protein, 4 aa, Artificial Sequence
  Synthetic Polypeptide
GGSG

SEQ ID NO: 14 - protein, 12 aa, Artificial Sequence
  Synthetic Polypeptide
GGSGGGSGGGSG

SEQ ID NO: 15 - protein, 15 aa, Artificial Sequence
  Synthetic Polypeptide
EAAAKEAAAKEAAAK

SEQ ID NO: 16 - protein, 20 aa, Artificial Sequence
  Synthetic Polypeptide
EAAAREAAAREAAAREAAAR

SEQ ID NO: 17 - protein, 17 aa, Artificial Sequence
  Synthetic Polypeptide
GGGGSGGGGSGGGGSAS

SEQ ID NO: 18 - protein, 9 aa, Artificial Sequence
  Synthetic Polypeptide
GGGGAGGGG

SEQ ID NO: 19 - skipped

SEQ ID NO: 20 - protein, 6 aa, Artificial Sequence
  Synthetic Polypeptide
GSGSGS

SEQ ID NO: 21 - protein, 10 aa, Artificial Sequence
  Synthetic Polypeptide
GSGSGSGSGS

SEQ ID NO: 22 - protein, 7 aa, Artificial Sequence
  Synthetic Polypeptide
GGGGSAS

SEQ ID NO: 23 - protein, 20 aa, Artificial Sequence
  Synthetic Polypeptide
APAPAPAPAPAPAPAPAPAP

SEQ ID NO: 24 - protein, 4 aa, Artificial Sequence
  Synthetic Polypeptide
CPPC

SEQ ID NO: 25 - protein, 5 aa, Artificial Sequence
  Synthetic Polypeptide
GGGGS

SEQ ID NO: 26 - protein, 10 aa, Artificial Sequence
  Synthetic Polypeptide
GGGGSGGGGS

SEQ ID NO: 27 - protein, 15 aa, Artificial Sequence
  Synthetic Polypeptide
GGGGSGGGGSGGGGS

SEQ ID NO: 28 - protein, 20 aa, Artificial Sequence
  Synthetic Polypeptide
GGGGSGGGGSGGGGSGGGGS

SEQ ID NO: 29 - protein, 25 aa, Artificial Sequence
  Synthetic Polypeptide
GGGGSGGGGSGGGGSGGGGSGGGGS

SEQ ID NO: 30 - protein, 30 aa, Artificial Sequence
  Synthetic Polypeptide
GGGGSGGGGSGGGGSGGGGSGGGGSGGGGS

SEQ ID NO: 31 - protein, 35 aa, Artificial Sequence
  Synthetic Polypeptide
GGGGSGGGGSGGGGSGGGGSGGGGSGGGGSGGGGS

SEQ ID NO: 32 - protein, 40 aa, Artificial Sequence
  Synthetic Polypeptide
GGGGSGGGGSGGGGSGGGGSGGGGSGGGGSGGGGSGGGGS

SEQ ID NO: 33 - protein, 16 aa, Artificial Sequence
  Synthetic Polypeptide
GGSGGSGGGGSGGGGS

SEQ ID NO: 34 - protein, 8 aa, Artificial Sequence
  Synthetic Polypeptide
GGGGGGGG

SEQ ID NO: 35 - protein, 6 aa, Artificial Sequence
  Synthetic Polypeptide
GGGGGG

SEQ ID NO: 36 - protein, 5 aa, Artificial Sequence
  Synthetic Polypeptide
EAAAK

SEQ ID NO: 37 - protein, 10 aa, Artificial Sequence
  Synthetic Polypeptide
EAAAKEAAAK

SEQ ID NO: 38 - protein, 15 aa, Artificial Sequence
  Synthetic Polypeptide
EAAAKEAAAKEAAAK

SEQ ID NO: 39 - protein, 12 aa, Artificial Sequence
  Synthetic Polypeptide
AEAAAKEAAAKA

SEQ ID NO: 40 - protein, 17 aa, Artificial Sequence
  Synthetic Polypeptide
AEAAAKEAAAKEAAAKA

SEQ ID NO: 41 - protein, 22 aa, Artificial Sequence
  Synthetic Polypeptide
AEAAAKEAAAKEAAAKEAAAKA

SEQ ID NO: 42 - protein, 27 aa, Artificial Sequence
  Synthetic Polypeptide
AEAAAKEAAAKEAAAKEAAAKEAAAKA

SEQ ID NO: 43 - protein, 46 aa, Artificial Sequence
  Synthetic Polypeptide
AEAAAKEAAAKEAAAKEAAAKALEAEAAAKEAAAKEAAAKEAAAKA

SEQ ID NO: 44 - protein, 5 aa, Artificial Sequence
  Synthetic Polypeptide
PAPAP

SEQ ID NO: 45 - protein, 18 aa, Artificial Sequence
  Synthetic Polypeptide
KESGSVSSEQLAQFRSLD

SEQ ID NO: 46 - protein, 12 aa, Artificial Sequence
  Synthetic Polypeptide
GSAGSAAGSGEF

SEQ ID NO: 47 - protein, 5 aa, Artificial Sequence
  Synthetic Polypeptide
GGGSE

SEQ ID NO: 48 - protein, 5 aa, Artificial Sequence
  Synthetic Polypeptide
GSESG

SEQ ID NO: 49 - protein, 5 aa, Artificial Sequence
  Synthetic Polypeptide
GSEGS

SEQ ID NO: 50 - protein, 35 aa, Artificial Sequence
  Synthetic Polypeptide
GEGGSGEGSSGEGSSSEGGGSEGGGSEGGGSEGGS

SEQ ID NO: 51 - protein, 234 aa, Artificial Sequence
  Synthetic Polypeptide
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 52 - protein, 234 aa, Artificial Sequence
  Synthetic Polypeptide
SKYGPPCPSCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 53 - protein, 234 aa, Artificial Sequence
  Synthetic Polypeptide
SKYGPPCPSCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 54 - protein, 234 aa, Artificial Sequence
  Synthetic Polypeptide
SKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 55 - protein, 234 aa, Artificial Sequence
  Synthetic Polypeptide
SKYGPPCPPCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 56 - protein, 234 aa, Artificial Sequence
  Synthetic Polypeptide
SKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 57 - protein, 343 aa, Artificial Sequence
  Synthetic Sequence
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 58 - protein, 215 aa, Artificial Sequence
  Synthetic Sequence
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 59 - protein, 133 aa, Artificial Sequence
  Synthetic Sequence
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 60 - protein, 792 aa, Artificial Sequence
  Synthetic Sequence
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 61 - protein, 710 aa, Artificial Sequence
  Synthetic Sequence
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 62 - protein, 182 aa, Artificial Sequence
  Synthetic Polypeptide
LQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 63 - protein, 759 aa, Artificial Sequence
  Synthetic Polypeptide
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV